(19) United States
(12) Patent Application Publication
(10) Pub. No.: US 2012/0096584 A1
(43) Pub. Date: Apr. 19, 2012

(54) NUCLEOTIDE SEQUENCES AND POLYPEPTIDES ENCODED THEREBY USEFUL FOR MODIFYING PLANT CHARACTERISTICS

(75) Inventors: Nickolai ALEXANDROV, Thousand Oaks, CA (US); Vyacheslav Brover, Simi Valley, CA (US); Kenneth A. Feldmann, Tucson, AZ (US); Timothy J. Swaller, Thousand Oaks, CA (US)

(73) Assignee: CERES, INC., Thousand Oaks, CA (US)

(21) Appl. No.: 13/235,320

(22) Filed: Sep. 16, 2011

Related U.S. Application Data
(63) Continuation of application No. 11/787,902, filed on Apr. 17, 2007, now abandoned.
(60) Provisional application No. 60/792,722, filed on Apr. 17, 2006.

(51) Int. Cl. A01H 5/00; C07H 21/02; C12N 15/82; A01H 3/04; C07K 14/415; C12Q 1/68; G01N 33/53; A01H 5/10; C12N 15/29; C12N 5/10

(52) U.S. Cl. 800/278; 536/23.6; 536/24.5; 435/320.1; 435/419; 530/370; 435/468; 435/6.11; 436/501; 800/298

(57) ABSTRACT

The present invention relates to isolated nucleic acid molecules and their corresponding encoded polypeptides able confer the trait of modulated low light sensitivity and modulated flowering time. The present invention further relates to the use of these nucleic acid molecules and polypeptides in making transgenic plants, plant cells, plant materials or seeds of a plant having such modulated growth or phenotype characteristics that are altered with respect to wild type plants grown under similar conditions.

This application is a Continuation of co-pending application Ser. No. 11/787,902 filed on Apr. 17, 2007 and for which priority is claimed under 35 U.S.C. §119(e) on U.S. Provisional Application No. 60/792,722 filed on Apr. 17, 2006, the entire contents of each of which are hereby incorporated by reference.

### FIELD OF THE INVENTION

The present invention relates to isolated polynucleotides, polypeptides encoded thereby, and the use of those products for making transgenic plants.

### BACKGROUND OF THE INVENTION

There are more than 300,000 species of plants. They show a wide diversity of forms, ranging from delicate liverworts, adapted for life in a damp habitat, to cacti, capable of surviving in the desert. The plant kingdom includes herbaceous plants, such as corn, whose life cycle is measured in months, to the giant redwood tree, which can live for thousands of years. This diversity reflects the adaptations of plants to survive in a wide range of habitats. This is seen most clearly in the flowering plants (phylum Angiospermophyta), which are the most numerous, with over 250,000 species. They are also the most widespread, being found from the tropics to the arctic.

The process of plant breeding involving man's intervention in natural breeding and selection is some 20,000 years old. It has produced remarkable advances in adapting existing species to serve new purposes. The world's economics was largely based on the successes of agriculture for most of these 20,000 years.

Plant breeding involves choosing parents, making crosses to allow recombination of gene (alleles) and searching for and selecting improved forms. Success depends on the genes/alleles available, the combinations required and the ability to create and find the correct combinations necessary to give the desired properties to the plant. Molecular genetics technologies are now capable of providing new genes, new alleles and the means of creating and selecting plants with the new, desired characteristics.

To summarize, molecular genetic technologies provide the ability to modulate and manipulate growth, development and biochemistry of the entire plant as well as at the cell, tissue and organ levels. Thus, plant morphology, development and biochemistry are altered to maximize or minimize the desired plant trait.

### SUMMARY OF THE INVENTION

The present invention, therefore, relates to isolated polynucleotides, polypeptides encoded thereby, and the use of those products for making transgenic organisms, such as plants, bacteria, yeast, fungi and mammals, depending upon the desired characteristics.

In the field of agriculture and forestry efforts are constantly being made to produce plants with improved characteristics, such as increased overall yield or increased yield of biomass or chemical components, in particular in order to guarantee the supply of the constantly increasing world population with food and to guarantee the supply of reproducible raw materials. Conventionally, people try to obtain plants with an increased yield by breeding, but this is time-consuming and labor-intensive. Furthermore, appropriate breeding programs must be performed for each relevant plant species.

Over the last two decades, progress has been made by the genetic manipulation of plants. That is, by introducing into plants recombinant nucleic acid molecules and expressing them as exogenous genes or using them to silence endogenous genes within these plants. Such approaches have the advantage of not usually being limited to one plant species, but being transferable to other plant species and other organisms as well. EP-A 0 511 979, for example, discloses that the expression of a prokaryotic asparagine synthetase in plant cells inter alia leads to an increase in biomass production. Similarly, WO 96/21737 describes the production of plants with increased yield from the expression of deregulated or unregulated fructose-1,6-bisphosphatase due to an increased rate of the photosynthesis. Nevertheless, there is still a need for generally applicable processes that lead to improved characteristics (such as yield) in relevant plants associated with a wide array of industrial purposes.

### DETAILED DESCRIPTION OF THE INVENTION

### 1. Definitions

The following terms are utilized throughout this application:

Domain: Domains are fingerprints or signatures that can be used to characterize protein families and/or parts of proteins. Such fingerprints or signatures can comprise conserved (1) primary sequence, (2) secondary structure, and/or (3) three-dimensional conformation. Generally, each domain has been associated with either a family of proteins or motifs. Typically, these families and/or motifs have been correlated with specific in-vitro and/or in-vivo activities. A domain can be any length, including the entirety of the sequence of a protein. Detailed descriptions of the domains, associated families and motifs, and correlated activities of the polypeptides of the instant invention are described below. Usually, the polypeptides with designated domain(s) can exhibit at least one activity that is exhibited by any polypeptide that comprises the same domain(s). Domains also define areas of non-coding sequences such as promoters and miRNAs.
Endogenous: The term “endogenous,” within the context of the current invention refers to any polynucleotide, polypeptide or protein sequence which is a natural part of a cell or organism regenerated from said cell.
Exogenous: “Exogenous,” as referred to within, is any polynucleotide, polypeptide or protein sequence, whether chimeric or not, that is initially or subsequently introduced into the genome of an individual host cell or the organism regenerated from said host cell by any means other than by a sexual cross. Examples of means by which this can be accomplished are described below, and include Agrobacterium-mediated transformation (of dicots—e.g. Salomon et al. (1984) EMBO J. 3:141; Herrera-Estrella et al. (1983) EMBO J. 2:987; of monocots, representative papers are those by Escudero et al. (1996) Plant J. 10:355; Ishida et al. (1996) Nature Biotechnology 14:745; May et al. (1995) Bio/Technology 13:486), biolistic methods (Armaleo et al. (1990) Current Genetics 17:97), electroporation, in planta techniques, and the like. The term “exogenous” as used herein is also intended to encompass inserting a naturally found element into a non-naturally found location.
Functionally Comparable Proteins or Functional Homologs: This phrase describes a set of proteins that perform similar functions within an organism. By definition, perturbation of an individual protein within that set (through misexpression or mutation, for example) is expected to confer a similar phenotype as compared to perturbation of any other individual protein. Such proteins typically share sequence similarity resulting in similar biochemical activity. Within this definition, homologs, orthologs and paralogs are considered to be functionally comparable.
Functionally comparable proteins will give rise to the same characteristic to a similar, but not necessarily the same, degree. Typically, comparable proteins give the same characteristics where the quantitative measurement due to one of the comparables is at least 20% of the other; more typically, between 30 to 40%; even more typically, between 50-60%; even more typically between 70 to 80%; even more typically between 90 to 100% of the other.
Gene: The term “gene,” as used in the context of the current invention, encompasses all regulatory and coding sequence contiguously associated with a single hereditary unit with a genetic function. Genes can include non-coding sequences that modulate the genetic function that include, but are not limited to, those that specify polyadenylation, transcriptional regulation, DNA conformation, chromatin conformation, extent and position of base methylation and binding sites of proteins that control all of these. Genes comprised of “exons” (coding sequences), which may be interrupted by “introns” (non-coding sequences), encode proteins. A gene's genetic function may require only RNA expression or protein production, or may only require binding of proteins and/or nucleic acids without associated expression. In certain cases, genes adjacent to one another may share sequence in such a way that one gene will overlap the other. A gene can be found within the genome of an organism, artificial chromosome, plasmid, vector, etc., or as a separate isolated entity.
Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.
Homologous gene: In the current invention, “homologous gene” refers to a gene that shares sequence similarity with the gene of interest. This similarity may be in only a fragment of the sequence and often represents a functional domain such as, examples including without limitation a DNA binding domain, a domain with tyrosine kinase activity, or the like. The functional activities of homologous genes are not necessarily the same.
Misexpression: The term “misexpression” refers to an increase or a decrease in the transcription of a coding region into a complementary RNA sequence as compared to the parental wild-type. This term also encompasses expression of a gene or coding region for a different time period as compared to the wild-type and/or from a non-natural location within the plant genome.
Native: As used herein, the term “native” refers to those sequences that are contiguous and/or operably linked in a wildtype plant.
Percentage of sequence identity: As used herein, the term “percent sequence identity” refers to the degree of identity between any given query sequence and a subject sequence. A subject sequence typically has a length that is from about 80 percent to 250 percent of the length of the query sequence, e.g., 82, 85, 87, 89, 90, 93, 95, 97, 99, 100, 105, 110, 115, or 120, 130, 140, 150, 160, 170, 180, 190, 200, 210, 220, 230, 240, or 250 percent of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chenna et al. (2003) Nucleic Acids Res. 31(13):3497-500. Optimal alignment of sequences for comparison may be conducted by the local homology algorithm of Smith and Waterman (1981) Add. APL. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85: 2444, by computerized implementations of algorithms such as GAP, BESTFIT, BLAST, PASTA, and TFASTA (Wisconsin Genetics Software Package, Genetics Computer Group (GCG), 575 Science Dr., Madison, Wis.) or by inspection. Typically, the default values of 5.00 for gap weight and 0.30 for gap weight length are used.

ClustalW calculates the best match between a query and one or more subject sequences, and aligns them so that identities, similarities and differences can be determined Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For an alignment of multiple nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher website and at the European Bioinformatics Institute website on the World Wide Web.

To determine a percent identity for polypeptide or nucleic acid sequences between a query and a subject sequence, the sequences are aligned using Clustal W and the number of identical matches in the alignment is divided by the query length, and the result is multiplied by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “substantial sequence identity” between polynucleotide or polypeptide sequences refers to polynucleotide or polypeptide comprising a sequence that has at least 80% sequence identity, preferably at least 85%, more preferably at least 90% and most preferably at least 95%, even more preferably, at least 96%, 97%, 98% or 99% sequence identity compared to a reference sequence using the programs.

Regulatory Sequence: The term “regulatory sequence,” as used in the current invention, refers to any nucleotide sequence that influences transcription or translation initiation and rate, and stability and/or mobility of the transcript or polypeptide product. Regulatory sequences include, but are not limited to, promoters, promoter control elements, protein binding sequences, 5′ and 3′ UTRs, transcriptional start site, termination sequence, polyadenylation sequence, introns, certain sequences within a coding sequence, etc.
Stringency: “Stringency,” as used herein is a function of nucleic acid molecule probe length, nucleic acid molecule probe composition (G+C content), salt concentration, organic solvent concentration and temperature of hybridization and/or wash conditions. Stringency is typically measured by the parameter Tₘ, which is the temperature at which 50% of the complementary nucleic acid molecules in the hybridization assay are hybridized, in terms of a temperature differential from Tₘ. High stringency conditions are those providing a condition of Tₘ−5° C. to Tₘ−10° C. Medium or moderate stringency conditions are those providing Tₘ−20° C. to Tₘ−29° C. Low stringency conditions are those providing a condition of Tₘ−40° C. to Tₘ−48° C. The relationship between hybridization conditions and Tₘ (in ° C.) is expressed in the mathematical equation:

Tₘ=81.5−16.6(log₁₀[Na⁺])+0.41(% G+C)−(600/N) (I)

where N is the number of nucleotides of the nucleic acid molecule probe. This equation works well for probes 14 to 70 nucleotides in length that are identical to the target sequence. The equation below, for Tₘ of DNA-DNA hybrids, is useful for probes having lengths in the range of 50 to greater than 500 nucleotides, and for conditions that include an organic solvent (formamide):

Tₘ=81.5+16.6 log {[Na⁺]/(1+0.7[Na⁺])}+0.41(% G+C)−500/L 0.63(% formamide) (II)

where L represents the number of nucleotides in the probe in the hybrid (Bonner et al. (1973) J. Mol. Biol. 81:123). The Tₘ of Equation II is affected by the nature of the hybrid: for DNA-RNA hybrids, Tₘ is 10-15° C. higher than calculated; for RNA-RNA hybrids, Tₘ is 20-25° C. higher. Because the Tₘ decreases about 1° C. for each 1% decrease in homology when a long probe is used (Frischauf et al. (1983) J. Mol. Biol, 170: 827-842), stringency conditions can be adjusted to favor detection of identical genes or related family members.

Equation II is derived assuming the reaction is at equilibrium. Therefore, hybridizations according to the present invention are most preferably performed under conditions of probe excess and allowing sufficient time to achieve equilibrium. The time required to reach equilibrium can be shortened by using a hybridization buffer that includes a hybridization accelerator such as dextran sulfate or another high volume polymer.

Stringency can be controlled during the hybridization reaction, or after hybridization has occurred, by altering the salt and temperature conditions of the wash solutions. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

Substantially free of: A composition containing A is “substantially free of” B when at least 85% by weight of the total A+B in the composition is A. Preferably, A comprises at least about 90% by weight of the total of A+B in the composition, more preferably at least about 95% or even 99% by weight. For example, a plant gene or DNA sequence can be considered substantially free of other plant genes or DNA sequences.
Translational start site: In the context of the current invention, a “translational start site” is usually an ATG in the cDNA transcript, more usually the first ATG. A single cDNA, however, may have multiple translational start sites.
Transcription start site: “Transcription start site” is used in the current invention to describe the point at which transcription is initiated. This point is typically located about 25 nucleotides downstream from a TFIID binding site, such as a TATA box. Transcription can initiate at one or more sites within the gene, and a single gene may have multiple transcriptional start sites, some of which may be specific for transcription in a particular cell-type or tissue.
Untranslated region (UTR): A “UTR” is any contiguous series of nucleotide bases that is transcribed, but is not translated. These untranslated regions may be associated with particular functions such as increasing mRNA message stability. Examples of UTRs include, but are not limited to polyadenylation signals, terminations sequences, sequences located between the transcriptional start site and the first exon (5′ UTR) and sequences located between the last exon and the end of the mRNA (3′ UTR).
Variant: The term “variant” is used herein to denote a polypeptide or protein or polynucleotide molecule that differs from others of its kind in some way. For example, polypeptide and protein variants can consist of changes in amino acid sequence and/or charge and/or post-translational modifications (such as glycosylation, etc).

### 2. Important Characteristics of the Polynucleotides of the Invention

The genes and polynucleotides of the present invention are of interest because when they are misexpressed (i.e. when over expressed at a non-natural location or in an increased amount) or when they allow silencing of endogenous genes, they produce plants with important modified characteristics as discussed below. These traits can be used to exploit or maximize plant products or to minimize undesirable characteristics. For example, an increase in plant height is beneficial in species grown or harvested for their main stem or trunk, such as ornamental cut flowers, fiber crops (e.g. flax, kenaf, hesperaloe, hemp) and wood producing trees. Increase in inflorescence thickness is also desirable for some ornamentals, while increases in the number, shape and size of leaves can lead to increased production/harvest from leaf crops such as lettuce, spinach, cabbage, swich grass and tobacco. Likewise, a decrease in plant height is beneficial in species that are particularly susceptible to lodging or uprooting due to wind stress.

The polynucleotides and polypeptides of the invention were isolated from different plant species as noted in the Sequence Listing. The polynucleotides and polypeptides are useful to confer on transgenic plants the properties identified for each sequence in the relevant portion (miscellaneous feature section) of the Sequence Listing. The miscellaneous feature section of the sequence listing contains, for each sequence, a description of the domain or other characteristic from which the sequence has the function known in the art for other sequences. Some identified domains are indicated with “PFam Name”, signifying that the pfam name and description can be found in the pfam database available via the internet. Other domains are indicated by reference to a “GI Number” from the public sequence database maintained by GenBank under the NCBI, including the non-redundant (NR) database.

The sequences of the invention can be applied to substrates for use in microarray applications such as, but not limited to, assays of global gene expression under varying development and growth conditions. The microarrays are also used for diagnostic or forensic purposes. Arrays can be produced using different procedures such as those from Affymetrix or Agilent. Protocols for these procedures can be obtained from these companies or found via the internet.

The polynucleotides, or fragments thereof, can also be used as probes and primers. Probe length varies depending on the application. For use as primers, probes are 12-40 nucleotides, preferably 18-30 nucleotides long. For use in mapping, probes are preferably 50 to 500 nucleotides, preferably 100-250 nucleotides long. For Southern hybridizations, probes as long as several kilobases are used.

The probes and/or primers are produced by synthetic procedures such as the triester method of Matteucci et al. (1981) J. Am. Chem. Soc. 103:3185 or according to Urdea et al. (1981) Proc. Natl. Acad. 80:7461 or using commercially available automated oligonucleotide synthesizers.

The polynucleotides of the invention can be utilized in a number of methods known to those skilled in the art as probes and/or primers to isolate and detect polynucleotides including, without limitation: Southerns, Northerns, Branched DNA hybridization assays, polymerase chain reaction microarray assays and variations thereof. Specific methods given by way of examples, and discussed below include:

Hybridization

Methods of Mapping

Southern Blotting

Isolating cDNA from Related Organisms

Isolating and/or Identifying Homologous and Orthologous Genes.

Also, the nucleic acid molecules of the invention can be used in other methods, such as high density oligonucleotide hybridizing assays, described, for example, in U.S. Pat. Nos. 6,004,753 and 5,945,306.

The polynucleotides or fragments thereof of the present invention can be used as probes and/or primers for detection and/or isolation of related polynucleotide sequences through hybridization. Hybridization of one nucleic acid to another constitutes a physical property that defines the polynucleotide of the invention and the identified related sequences. Also, such hybridization imposes structural limitations on the pair. A good general discussion of the factors for determining hybridization conditions is provided by Sambrook et al. (“Molecular Cloning, a Laboratory Manual, 2nd ed., c. 1989 by Cold Spring Harbor Laboratory Press, Cold Spring Harbor, N.Y.; see esp., chapters 11 and 12). Additional considerations and details of the physical chemistry of hybridization are provided by G. H. Keller and M. M. Manak “DNA Probes”, 2^{nd} Ed. pp. 1-25, c. 1993 by Stockton Press, New York, N.Y.

When using the polynucleotides to identify homologous genes in other species, the practitioner will preferably adjust the amount of target DNA of each species so that, as nearly as is practical, the same number of genome equivalents are present for each species examined. This prevents faint signals from species having large genomes, and thus small numbers of genome equivalents per mass of DNA, from erroneously being interpreted as absence of the corresponding gene in the genome.

The probes and/or primers of the instant invention can also be used to detect or isolate nucleotides that are “identical” to the probes or primers. Two nucleic acid sequences or polypeptides are said to be “identical” if the sequence of nucleotides or amino acid residues, respectively, in the two sequences is the same when aligned for maximum correspondence as described below.

Isolated polynucleotides within the scope of the invention also include allelic variants of the specific sequences presented in the Sequence Listing. The probes and/or primers of the invention are also used to detect and/or isolate polynucleotides exhibiting at least 80% sequence identity with the sequences of the Sequence Listing or fragments thereof. Related polynucleotide sequences can also be identified according to the methods described in U.S. Patent Publication 20040137466A1, dated Jul. 15, 2004 to Jofuku et al.

With respect to nucleotide sequences, degeneracy of the genetic code provides the possibility to substitute at least one nucleotide of the nucleotide sequence of a gene with a different nucleotide without changing the amino acid sequence of the polypeptide. Hence, the DNA of the present invention also has any base sequence that has been changed from a sequence in the Sequence Listing by substitution in accordance with degeneracy of genetic code. References describing codon usage include: Cards et al. (1998) J. Mol. Evol. 46: 45 and Fennoy et al. (1993) Nucl. Acids Res. 21(23): 5294.

The polynucleotides of the invention are also used to create various types of genetic and physical maps of the genome of the plant species listed in the Sequence Listing. Some are absolutely associated with particular phenotypic traits, allowing construction of gross genetic maps. Creation of such maps is based on differences or variants, generally referred to as polymorphisms, between different parents used in crosses. Common methods of detecting polymorphisms that can be used are restriction fragment length polymorphisms (RFLPs, single nucleotide polymorphisms (SNPs) or simple sequence repeats (SSRs).

The use of RFLPs and of recombinant inbred lines for such genetic mapping is described for Arabidopsis by Alonso-Blanco et al. (Methods in Molecular Biology, vol. 82, “Arabidopsis Protocols”, pp. 137-146, J. M. Martinez-Zapater and J. Salinas, eds., c. 1998 by Humana Press, Totowa, N.J.) and for corn by Burr (“Mapping Genes with Recombinant Inbreds”, pp. 249-254. In Freeling, M. and V. Walbot (Ed.), The Maize Handbook, c. 1994 by Springer-Verlag New York, Inc.: New York, N.Y., USA; Berlin Germany; Burr et al. Genetics (1998) 118: 519; Gardiner, J. et al. (1993) Genetics 134: 917). This procedure, however, is not limited to plants and is used for other organisms (such as yeast) or for individual cells.

The polynucleotides of the present invention are also used for simple sequence repeat (SSR) mapping. Rice SSR mapping is described by Morgante et al. (The Plant Journal (1993) 3: 165), Panaud et al. (Genome (1995) 38: 1170); Senior et al. (Crop Science (1996) 36: 1676), Taramino et al. (Genome (1996) 39: 277) and Ahn et al. (Molecular and General Genetics (1993) 241: 483-90). SSR mapping is achieved using various methods. In one instance, polymorphisms are identified when sequence specific probes contained within a polynucleotide flanking an SSR are made and used in polymerase chain reaction (PCR) assays with template DNA from two or more individuals of interest. Here, a change in the number of tandem repeats between the SSR-flanking sequences produces differently sized fragments (U.S. Pat. No. 5,766,847). Alternatively, polymorphisms are identified by using the PCR fragment produced from the SSR-flanking sequence specific primer reaction as a probe against Southern blots representing different individuals (U. H. Refseth et al. (1997) Electrophoresis 18: 1519).

The polynucleotides of the invention can further be used to identify certain genes or genetic traits using, for example, known AFLP technologies, such as in EP0534858 and U.S. Pat. No. 5,878,215.

The polynucleotides of the present invention are also used for single nucleotide polymorphism (SNP) mapping.

Genetic and physical maps of crop species have many uses. For example, these maps are used to devise positional cloning strategies for isolating novel genes from the mapped crop species. In addition, because the genomes of closely related species are largely syntenic (i.e. they display the same ordering of genes within the genome), these maps are used to isolate novel alleles from relatives of crop species by positional cloning strategies.

The various types of maps discussed above are used with the polynucleotides of the invention to identify Quantitative Trait Loci (QTLs). Many important crop traits, such as the solids content of tomatoes, are quantitative traits and result from the combined interactions of several genes. These genes reside at different loci in the genome, often times on different chromosomes, and generally exhibit multiple alleles at each locus. The polynucleotides of the invention are used to identify QTLs and isolate specific alleles as described by de Vicente and Tanksley (Genetics (1993) 134:585). Once a desired allele combination is identified, crop improvement is accomplished either through biotechnological means or by directed conventional breeding programs (for review see Tanksley and McCouch (1997) Science 277:1063). In addition to isolating QTL alleles in present crop species, the polynucleotides of the invention are also used to isolate alleles from the corresponding QTL of wild relatives.

In another embodiment, the polynucleotides are used to help create physical maps of the genome of the plant species mentioned in the Sequence Listing and related species thereto. Where polynucleotides are ordered on a genetic map, as described above, they are used as probes to discover which clones in large libraries of plant DNA fragments in YACs, BACs, etc. contain the same polynucleotide or similar sequences, thereby facilitating the assignment of the large DNA fragments to chromosomal positions. Subsequently, the large BACs, YACs, etc. are ordered unambiguously by more detailed studies of their sequence composition (e.g. Marra et al. (1997) Genomic Research 7:1072-1084) and by using their end or other sequences to find the identical sequences in other cloned DNA fragments. The overlapping of DNA sequences in this way allows building large contigs of plant sequences to be built that, when sufficiently extended, provide a complete physical map of a chromosome. Sometimes the polynucleotides themselves provide the means of joining cloned sequences into a contig. All scientific and patent publications cited in this paragraph are hereby incorporated by reference.

U.S. Pat. Nos. 6,287,778 and 6,500,614, both hereby incorporated by reference, describe scanning multiple alleles of a plurality of loci using hybridization to arrays of oligonucleotides. These techniques are useful for each of the types of mapping discussed above.

Following the procedures described above and using a plurality of the polynucleotides of the present invention, any individual is genotyped. These individual genotypes are used for the identification of particular cultivars, varieties, lines, ecotypes and genetically modified plants or can serve as tools for subsequent genetic studies involving multiple phenotypic traits.

Identification and isolation of orthologous genes from closely related species and alleles within a species is particularly desirable because of their potential for crop improvement. Many important crop traits result from the combined interactions of the products of several genes residing at different loci in the genome. Generally, alleles at each of these loci make quantitative differences to the trait. Once a more favorable allele combination is identified, crop improvement is accomplished either through biotechnological means or by directed conventional breeding programs (Tanksley et al. (1997) Science 277:1063).

### 4. Use of the Genes to Make Transgenic Plants

To use the sequences of the present invention or a combination of them or parts and/or mutants and/or fusions and/or variants of them, recombinant DNA constructs are prepared which comprise the polynucleotide sequences of the invention inserted into a vector, and which are suitable for transformation of plant cells. The construct is made using standard recombinant DNA techniques (Sambrook et al. 1989) and is introduced to the species of interest by Agrobacterium-mediated transformation or by other means of transformation as referenced below.

The sequences of the present invention can be in sense orientation or in anti-sense orientation.

If a decrease in the transcription or translation product of an endogenous gene (gene silencing) is desired, the sequence of interest is transcribed as an antisense nucleic acid or an interfering RNA similar or identical to part of the endogenous gene. Antisense nucleic acids or interfering RNAs are about 10 nucleotides to about 2,500 nucleotides in length. For example, the nucleic acid of the present invention can be used as an antisense nucleic acid to its corresponding endogenous gene. Alternatively, the transcription product of a nucleic acid of the invention can be similar or identical to the sense coding sequence of its corresponding endogenous gene, but is an RNA that is unpolyadenylated, lacks a 5′ cap structure, or contains an unsplicable intron. The nucleic acid of the present invention in sense orientation can also be used as a partial or full-length coding sequence that results in inhibition of the expression of an endogenous polypeptide by co-suppression. Methods of co-suppression using a full-length cDNA sequence as well as a partial cDNA sequence are known in the art (see, for example, U.S. Pat. No. 5,231,020).

Alternatively, a nucleic acid can be transcribed into a ribozyme that affects expression of an mRNA (see U.S. Pat. No. 6,423,885). Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art (see, for example, U.S. Pat. No. 5,254,678). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92(13):6175-6179; de Feyter and Gaudron Methods in Molecular Biology, Vol. 74, Chapter 43, “Expressing Ribozymes in Plants”, Edited by Turner, P. C, Humana Press Inc., Totowa, N.J.). RNA endoribonucleases such as the one that occurs naturally in Tetrahymena thermophila and which have been described extensively by Cech and collaborators can also be useful (see, for example, U.S. Pat. No. 4,987,071).

A nucleic acid of the present invention can also be used for its transcription into an interfering RNA. Such an RNA can be one that can anneal to itself, for example a double stranded RNA having a stem-loop structure. One strand of the stem portion of a double stranded RNA can comprise a sequence that is similar or identical to the sense coding sequence of an endogenous polypeptide and that is about 10 nucleotides to about 2,500 nucleotides in length. Generally, the length of the nucleic acid sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA can comprise an antisense sequence of an endogenous polypeptide and can have a length that is shorter, the same as, or longer than the length of the corresponding sense sequence. The loop portion of a double stranded RNA can be from 10 nucleotides to 500 nucleotides in length, for example from 15 nucleotides to 100 nucleotides, from 20 nucleotides to 300 nucleotides or from 25 nucleotides to 400 nucleotides in length. The loop portion of the RNA can include an intron (see, for example the following publications: WO 98/53083; WO 99/32619; WO 98/36083; WO 99/53050; US 20040214330; US 20030180945; U.S. Pat. No. 5,034,323; U.S. Pat. No. 6,452,067; U.S. Pat. No. 6,777,588; U.S. Pat. No. 6,573,099 and U.S. Pat. No. 6,326,527). Interfering RNA also can be constructed as described in Brummell, et al. (2003) Plant J. 33:793-800.

The vector backbone for the recombinant constructs is any of those typical in the art such as plasmids (such as Ti plasmids), viruses, artificial chromosomes, BACs, YACs and PACs and vectors of the sort described by
(a) BAC: Shizuya et al. (1992) Proc. Natl. Acad. Sci. USA 89: 8794-8797; Hamilton et al. (1996) Proc. Natl. Acad. Sci. USA 93: 9975-9979;
(b) YAC: Burke et al. (1987) Science 236:806-812;
(c) PAC: Sternberg N. et al. (1990) Proc Natl Acad Sci USA. January; 87(1):103-7;
(d) Bacteria-Yeast Shuttle Vectors: Bradshaw et al. (1995) Nucl Acids Res 23: 4850-4856;
(e) Lambda Phage Vectors: Replacement Vector, e.g., Frischauf et al. (1983) J. Mol Biol 170: 827-842; or Insertion vector, e.g., Huynh et al., In: Glover N M (ed) DNA Cloning: A practical Approach, Vol. 1 Oxford: IRL Press (1985); T-DNA gene fusion vectors:Walden et al. (1990) Mol Cell Biol 1: 175-194; and
(g) Plasmid vectors: Sambrook et al., infra.

Typically, the construct comprises a vector containing a sequence of the present invention with any desired transcriptional and/or translational regulatory sequences, such as promoters, UTRs, and 3′ end termination sequences. Vectors can also include origins of replication, scaffold attachment regions (SARs), markers, homologous sequences, introns, etc. The vector may also comprise a marker gene that confers a selectable phenotype on plant cells. The marker may encode biocide resistance, particularly antibiotic resistance, such as resistance to kanamycin, G418, bleomycin, hygromycin, or herbicide resistance, such as resistance to chlorosulfuron, glyphosate or phosphinotricin.

A plant promoter fragment is used that directs transcription of the gene in all tissues of a regenerated plant and/or is a constitutive promoter. Alternatively, the plant promoter directs transcription of a sequence of the invention in a specific tissue (tissue-specific promoter) or is otherwise under more precise environmental control, such as chemicals, cold, heat, drought, salt and many others (inducible promoter).

If proper polypeptide production is desired, a polyadenylation region at the 3′-end of the coding region is typically included. The polyadenylation region is derived from the natural gene, from a variety of other plant genes, or from T-DNA, synthesized in the laboratory.

### Transformation

Techniques for transforming a wide variety of higher plant species are well known and described in the technical and scientific literature. See, e.g. Weising et al. (1988) Ann. Rev. Genet. 22:421 and Christou(1995) Euphytica, v. 85, n.1-3:13-27.

The person skilled in the art knows processes for the transformation of monocotyledonous and dicotyledonous plants. A variety of techniques are available for introducing DNA into a plant host cell. These techniques comprise transformation of plant cells by DNA injection, DNA electroporation, use of bolistics methods, protoplast fusion and via T-DNA using Agrobacterium tumefaciens or Agrobacterium rhizogenes, as well as further possibilities, or other bacterial hosts for Ti plasmid vectors. See for example, Broothaerts et al. (2005) Gene Transfer to Plants by Diverse Species of Bacteria, Nature, Vol. 433, pp. 629-633.

DNA constructs of the invention are introduced into the cell or the genome of the desired plant host by a variety of conventional techniques. For example, the DNA construct is introduced using techniques such as electroporation, microinjection and polyethylene glycol precipitation of plant cell protoplasts or protoplast fusion. Electroporation techniques are described in Fromm et al. (1985) Proc. Natl. Acad. Sci. USA 82:5824. Microinjection techniques are known in the art and well described in the scientific and patent literature. The plasmids do not have to fulfill specific requirements for use in DNA electroporation or DNA injection into plant cells. Simple plasmids such as pUC derivatives can be used.

The introduction of DNA constructs using polyethylene glycol precipitation is described in Paszkowski et al. (1984) EMBO J. 3:2717. Introduction of foreign DNA using protoplast fusion is described by Willmitzer (Willmitzer, L. (1993) Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basel-Cambridge).

Alternatively, the DNA constructs of the invention are introduced directly into plant tissue using ballistic methods, such as DNA particle bombardment. Ballistic transformation techniques are described in Klein et al. (1987) Nature 327:773. Introduction of foreign DNA using ballistics is described by Willmitzer (Willmitzer, L., 1993 Transgenic plants. In: Biotechnology, A Multi-Volume Comprehensive Treatise (H. J. Rehm, G. Reed, A. Pithier, P. Stadler, eds.), Vol. 2, 627-659, VCH Weinheim-New York-Basel-Cambridge).

DNA constructs are also introduced with the help of Agrobacteria. The use of Agrobacteria for plant cell transformation is extensively examined and sufficiently disclosed in the specification of EP-A 120 516, and in Hoekema (In: The Binary Plant Vector System Offsetdrukkerij Kanters B. V., Alblasserdam (1985), Chapter V), Fraley et al. (Crit. Rev. Plant. Sci. 4, 1-46) and DePicker et al. (EMBO J. 4 (1985), 277-287). Using this technique, the DNA constructs of the invention are combined with suitable T-DNA flanking regions and introduced into a conventional Agrobacterium tumefaciens host vector. The virulence functions of the Agrobacterium tumefaciens host direct the insertion of the construct and adjacent marker(s) into the plant cell DNA when the cell is infected by the bacteria (McCormac et al. (1997) Mol. Biotechnol. 8:199; Hamilton (1997) Gene 200:107; Salomon et al. (1984) EMBO J. 3:141; Herrera-Estrella et al. (1983) EMBO J. 2:987). Agrobacterium tumefaciens-mediated transformation techniques, including disarming and use of binary or co-integrate vectors, are well described in the scientific literature. See, for example Hamilton (1997) Gene 200:107; Müller et al. (1987) Mol. Gen. Genet. 207:171; Komari et al. (1996) Plant J. 10:165; Venkateswarlu et al. (1991) Biotechnology 9:1103 and Gleave (1992) Plant Mol. Biol. 20:1203; Graves and Goldman (1986) Plant Mol. Biol. 7:34 and Gould et al. (1991) Plant Physiology 95:426.

For plant cell T-DNA transfer of DNA, plant organs, e.g. infloresences, plant explants, plant cells that have been cultured in suspension or protoplasts are co-cultivated with Agrobacterium tumefaciens or Agrobacterium rhizogenes or other suitable T-DNA hosts. Whole plants are regenerated from the infected plant material or seeds generated from infected plant material using a suitable medium that contains antibiotics or biocides for the selection of transformed cells or by spraying the biocide on plants to select the transformed plants. Plants obtained in this way are then examined for the presence of the DNA introduced. The transformation of dicotyledonous plants via Ti-plasmid-vector systems and Agrobacterium tumefaciens is well established.

Monocotyledonous plants are also transformed by means of Agrobacterium based vectors (See Chan et al. (1993) Plant Mol. Biol. 22: 491-506; Hiei et al. (1994) Plant J. 6:271-282; Deng et al. (1990) Science in China 33:28-34; Wilmink et al. Plant (1992) Cell Reports 11:76-80; May et al. (1995) Bio/Technology 13:486-492; Conner and Domisse (1992) Int. J. Plant Sci. 153:550-555; Ritchie et al. (1993) Transgenic Res. 2:252-265). Maize transformation in particular is described in the literature (see, for example, WO95/06128, EP 0 513 849; EP 0 465 875; Fromm et al., (1990) Biotechnology 8:833-844; Gordon-Kamm et al. (1990) Plant Cell 2:603-618; Koziel et al. (1993) Biotechnology 11:194-200). In EP 292 435 and in Shillito et al. (Bio/Technology (1989) 7:581) fertile plants are obtained from a mucus-free, soft (friable) maize callus. Prioli and Söndahl (Bio/Technology (1989) 7, 589) also report regenerating fertile plants from maize protoplasts of the maize Cateto inbred line, Cat 100-1.

Other cereal species have also been successfully transformed, such as barley (Wan and Lemaux, see above; Ritala et al., see above) and wheat (Nehra et al. (1994) Plant J. 5, 285-297).

Alternatives to Agrobacterium transformation for plants are ballistics, protoplast fusion, electroporation of partially permeabilized cells and use of glass fibers (See Wan and Lemaux (1994) Plant Physiol. 104:37-48; Vasil et al. (1993) Bio/Technology 11:1553-1558; Ritala et al. (1994) Plant Mol. Biol. 24:317-325; Spencer et al. (1990) Theor. Appl. Genet. 79:625-631).

Introduced DNA is usually stable after integration into the plant genome and is transmitted to the progeny of the transformed cell or plant. Generally the transformed plant cell contains a selectable marker that makes the transformed cells resistant to a biocide or an antibiotic such as kanamycin, G 418, bleomycin, hygromycin, phosphinotricin or others. Therefore, the individually chosen marker should allow the selection of transformed cells from cells lacking the introduced DNA.

The transformed cells grow within the plant in the usual way (McCormick et al. (1986) Plant Cell Reports 5, 81-84) and the resulting plants are cultured normally. Transformed plant cells obtained by any of the above transformation techniques are cultured to regenerate a whole plant that possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences.

Plant regeneration from cultured protoplasts is described in Evans et al., Protoplasts Isolation and Culture in “Handbook of Plant Cell Culture,” pp. 124-176, MacMillan Publishing Company, New York, 1983; and Binding, Regeneration of Plants, Plant Protoplasts, pp. 21-73, CRC Press, Boca Raton, 1988. Regeneration also occurs from plant callus, explants, organs, or parts thereof. Such regeneration techniques are described generally in Klee et al. (1987) Ann. Rev. of Plant Phys. 38:467. Regeneration of monocots (rice) is described by Hosoyama et al. (Biosci. Biotechnol. Biochem. (1994) 58:1500) and by Ghosh et al. (J. Biotechnol. (1994) 32:1). Useful and relevant procedures for transient expression are also described in U.S. Application No. 60/537,070 filed on Jan. 16, 2004 and PCT Application No. PCT/US2005/001153 filed on Jan. 14, 2005.

After transformation, seeds are obtained from the plants and used for testing stability and inheritance. Generally, two or more generations are cultivated to ensure that the phenotypic feature is stably maintained and transmitted.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The nucleotide sequences according to the invention generally encode an appropriate protein from any organism, in particular from plants, fungi, bacteria or animals. The sequences preferably encode proteins from plants or fungi. Preferably, the plants are higher plants, in particular starch or oil storing useful plants, such as potato or cereals such as rice, maize, wheat, barley, rye, triticale, oat, millet, etc., as well as spinach, tobacco, sugar beet, soya, cotton etc.

In principle, the process according to the invention can be applied to any plant. Therefore, monocotyledonous as well as dicotyledonous plant species are particularly suitable. The process is preferably used with plants that are interesting for agriculture, horticulture, biomass for conversion, textile, plants as chemical factories and/or forestry.

Thus, the invention has use over a broad range of plants, preferably higher plants, pertaining to the classes of Angiospermae and Gymnospermae. Plants of the subclasses of the Dicotylodenae and the Monocotyledonae are particularly suitable. Dicotyledonous plants belong to the orders of the Magniolales, Illiciales, Laurales, Piperales Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. Monocotyledonous plants belong to the orders of the Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchidales. Plants belonging to the class of the Gymnospermae are Pinales, Ginkgoales, Cycadales and Gnetales.

Examples of species represented in these orders are tobacco, oilseed rape, sugar beet, potato, tomato, lettuce, cucumber, pepper, bean, pea, citrus fruit, apple, pear, berries, plum, melon, eggplant, cotton, soybean, sunflower, rose, poinsettia, petunia, guayule, cabbage, spinach, alfalfa, artichoke, corn, wheat, rye, barley, grasses such as switch grass or turf grass, millet, hemp, banana, poplar, eucalyptus trees, conifers.

### Characteristics of the Sequences, as Noted in the Sequence Listing

The modulated growth and phenotype characteristics for each of the sequences of the invention are noted by an entry in a “miscellaneous feature” section for each respective nucleic acid and/or polypeptide sequence in the Sequence Listing.

For some of the polynucleotides/polypeptides of the invention, the sequence listing further includes (in a “miscellaneous feature” section) an indication of any important identified domain(s) and the corresponding function of the domain(s) identified by comparison to the publicly available pfam database.

For some of the polynucleotides/polypeptides of the invention, the sequence listing further includes (in a “miscellaneous feature” section) an indication of important identified “phenotype” characteristic(s) of a polypeptide sequence and the corresponding function of the polypeptide sequence identified by comparison to the publicly available Swiss-Prot database.

Table 1 correlates the “Hit” sequence with the experimental observation noted for it in the “Observation” column and the “phenotype” which is present in the “Phenotype” column and the miscellaneous feature section of the Sequence Listing.

The “Phenotypes” listed in Table 1 and the Sequence Listing are listed and described below providing further explanation of how the sequences are used to effect phenotypic results in transgenic plants. Additional description and specific annotation of the “phenotypes” associated with some particular sequences in the Sequence Listing can be found in application Ser. Nos. 10/225,066 (US 2005/0160493), 11/479,226 (US 2007/0022495) and 10/374,780 (US 2006/0162006), all of which are hereby incorporated by reference in their entirety.

TABLE 1

| HIT_ID | OBSERVATION | PHENOTYPE |
|---|---|---|
| SEQ_ID_NO_10026 | | Useful for making plants with |
| | | modulated nitrogen use efficiency |
| SEQ_ID_NO_10048 | | Useful for making plants with |
| | | modulated cold sensitivity |
| SEQ_ID_NO_1016 | The plant flowers significantly late | Useful for making plants with |
| | compared to wild-type. | modulated flowering time |
| SEQ_ID_NO_10172 | | Useful for making plants with |
| | | modulated cold sensitivity |
| SEQ_ID_NO_10176 | The plant is abnormally small. | Useful for making plants with |
| | | modulated biomass |
| SEQ_ID_NO_10176 | Useful for making plants with | The sequence is a functional homolog |
| | modulated growth and phenotype | of SEQ ID NO 960 in the 10/225,066 |
| | characteristics | US patent application |
| SEQ_ID_NO_10176 | Useful for making plants with | The sequence is a functional homolog |
| | modulated growth and phenotype | of SEQ ID NO 874 in the 10/412,699 |
| | characteristics | US patent application |
| SEQ_ID_NO_10176 | Useful for making plants with | The sequence is a functional homolog |
| | modulated growth and phenotype | of SEQ ID NO 2604 in the 10/374,780 |
| | characteristics | US patent application |
| SEQ_ID_NO_10176 | Useful for making plants with | The sequence is a functional homolog |
| | modulated growth and phenotype | of SEQ ID NO 1769 in the 11/479,226 |
| | characteristics | US patent application |
| SEQ_ID_NO_10176 | Useful for making plants with | The sequence is a functional homolog |
| | modulated growth and phenotype | of SEQ ID NO 1765 in the 11/479,226 |
| | characteristics | US patent application |
| SEQ_ID_NO_10176 | Useful for making plants with | The sequence is a functional homolog |
| | modulated growth and phenotype | of SEQ ID NO 1761 in the 11/479,226 |
| | characteristics | US patent application |
| SEQ_ID_NO_10184 | | Useful for making plants with altered |
| | | terpenoid content |
| SEQ_ID_NO_10184 | | Useful for making plants with altered |
| | | Lignin content |
| SEQ_ID_NO_10192 | | Useful for making plants with altered |
| | | cell wall content and/or composition |
| SEQ_ID_NO_10198 | | Useful for making plants with altered |
| | | Lignin content |
| SEQ_ID_NO_10242 | | Useful for making plants with |
| | | modulated nitrogen use efficiency |
| SEQ_ID_NO_10242 | The plant flowers significantly late | Useful for making plants with |
| | compared to wild-type. | modulated flowering time |
| SEQ_ID_NO_10242 | | Useful for making plants with altered |
| | | Lignin content |
| SEQ_ID_NO_10258 | | Useful for making plants with altered |
| | | Lignin content |
| SEQ_ID_NO_10258 | | Useful for making plants with altered |
| | | alkaloid content |
| SEQ_ID_NO_10270 | The plant is abnormally large. | Useful for making plants with |
| | | modulated biomass |
| SEQ_ID_NO_10278 | The plant flowers significantly late | Useful for making plants with |
| | compared to wild-type. | modulated flowering time |
| SEQ_ID_NO_10278 | No seed is being produced by the plant | Useful for biocontainment applications |
| SEQ_ID_NO_10304 | The plant is abnormally small. | Useful for making plants with |
| | | modulated biomass |
| SEQ_ID_NO_10326 | Useful for making plants with | The sequence is a functional homolog |
| | modulated growth and phenotype | of SEQ ID NO 382 in the 10/714,887 |
| | characteristics | US patent application |
| SEQ_ID_NO_10326 | Useful for making plants with | The sequence is a functional homolog |
| | modulated growth and phenotype | of SEQ ID NO 380 in the 11/435,388 |
| | characteristics | US patent application |
| SEQ_ID_NO_10326 | Useful for making plants with | The sequence is a functional homolog |
| | modulated growth and phenotype | of SEQ ID NO 380 in the 10/714,887 |
| | characteristics | US patent application |
| SEQ_ID_NO_10326 | Useful for making plants with | The sequence is a functional homolog |
| | modulated growth and phenotype | of SEQ ID NO 378 in the 11/435,388 |
| | characteristics | US patent application |
| SEQ_ID_NO_10326 | Useful for making plants with | The sequence is a functional homolog |
| | modulated growth and phenotype | of SEQ ID NO 378 in the 10/714,887 |
| | characteristics | US patent application |
| SEQ_ID_NO_10326 | Useful for making plants with | The sequence is a functional homolog |
| | modulated growth and phenotype | of SEQ ID NO 376 in the 11/435,388 |
| | characteristics | US patent application |
| SEQ_ID_NO_10326 | Useful for making plants with | The sequence is a functional homolog |
| | modulated growth and phenotype | of SEQ ID NO 376 in the 10/714,887 |
| | characteristics | US patent application |
| SEQ_ID_NO_10326 | Useful for making plants with | The sequence is a functional homolog |
| | modulated growth and phenotype | of SEQ ID NO 374 in the 11/435,388 |
| | characteristics | US patent application |
| SEQ_ID_NO_10326 | Useful for making plants with | The sequence is a functional homolog |
| | modulated growth and phenotype | of SEQ ID NO 2167 in the 10/666,642 |
| | characteristics | US patent application |
| SEQ_ID_NO_10326 | Useful for making plants with | The sequence is a functional homolog |
| | modulated growth and phenotype | of SEQ ID NO 2165 in the 10/666,642 |
| | characteristics | US patent application |
| SEQ_ID_NO_10326 | Useful for making plants with | The sequence is a functional homolog |
| | modulated growth and phenotype | of SEQ ID NO 2163 in the 10/666,642 |
| | characteristics | US patent application |
| SEQ_ID_NO_10326 | Useful for making plants with | The sequence is a functional homolog |
| | modulated growth and phenotype | of SEQ ID NO 2161 in the 10/666,642 |
| | characteristics | US patent application |
| SEQ_ID_NO_10326 | Useful for making plants with | The sequence is a functional homolog |
| | modulated growth and phenotype | of SEQ ID NO 1088 in the 10/374,780 |
| | characteristics | US patent application |
| SEQ_ID_NO_10326 | Useful for making plants with | The sequence is a functional homolog |
| | modulated growth and phenotype | of SEQ ID NO 1087 in the 10/374,780 |
| | characteristics | US patent application |
| SEQ_ID_NO_10326 | Useful for making plants with | The sequence is a functional homolog |
| | modulated growth and phenotype | of SEQ ID NO 1086 in the 10/374,780 |
| | characteristics | US patent application |
| SEQ_ID_NO_10372 | | Useful for making plants with |
| | | modulated nitrogen use efficiency |
| SEQ_ID_NO_10380 | The plant is abnormally small. | Useful for making plants with |
| | | modulated biomass |
| SEQ_ID_NO_10388 | The plant is abnormally large. | Useful for making plants with |
| | | modulated biomass |
| SEQ_ID_NO_10408 | | Useful for making plants with |
| | | modulated drought sensitivity |
| SEQ_ID_NO_10416 | | Useful for making plants with |
| | | modulated phosphate use efficiency |
| SEQ_ID_NO_10416 | | Useful for making plants with |
| | | modulated nitrogen use efficiency |
| SEQ_ID_NO_10420 | The plant flowers significantly earllier | Useful for making plants with |
| | than wild-type. | modulated flowering time |
| SEQ_ID_NO_10420 | | Useful for making plants with |
| | | modulated flowering time |
| SEQ_ID_NO_10420 | The plant is abnormally small. | Useful for making plants with |
| | | modulated biomass |
| SEQ_ID_NO_10420 | | Useful for making plants with |
| | | modulated biomass |
| SEQ_ID_NO_10440 | Useful for making plants with | The sequence is a functional homolog |
| | modulated growth and phenotype | of SEQ ID NO 960 in the 10/225,066 |
| | characteristics | US patent application |
| SEQ_ID_NO_10440 | Useful for making plants with | The sequence is a functional homolog |
| | modulated growth and phenotype | of SEQ ID NO 2604 in the 10/374,780 |
| | characteristics | US patent application |
| SEQ_ID_NO_10440 | Useful for making plants with | The sequence is a functional homolog |
| | modulated growth and phenotype | of SEQ ID NO 1769 in the 11/479,226 |
| | characteristics | US patent application |
| SEQ_ID_NO_10514 | | Useful for making plants with altered |
| | | cell wall content and/or composition |
| SEQ_ID_NO_10524 | The plant is abnormally small. | Useful for making plants with |
| | | modulated biomass |
| SEQ_ID_NO_10526 | The plant flowers significantly late | Useful for making plants with |
| | compared to wild-type. | modulated flowering time |
| SEQ_ID_NO_10526 | No seed is being produced by the plant | Useful for biocontainment applications |
| SEQ_ID_NO_10532 | | Useful for making plants with altered |
| | | Lignin content |
| SEQ_ID_NO_10532 | | Useful for making plants with altered |
| | | alkaloid content |
| SEQ_ID_NO_10550 | The plant is abnormally large. | Useful for making plants with |
| | | modulated biomass |
| SEQ_ID_NO_10588 | The plant flowers significantly earllier | Useful for making plants with |
| | than wild-type. | modulated flowering time |
| SEQ_ID_NO_10588 | | Useful for making plants with |
| | | modulated flowering time |
| SEQ_ID_NO_10588 | The plant is abnormally small. | Useful for making plants with |
| | | modulated biomass |
| SEQ_ID_NO_10588 | | Useful for making plants with |
| | | modulated biomass |
| SEQ_ID_NO_10604 | The plant flowers significantly earllier | Useful for making plants with |
| | than wild-type. | modulated flowering time |
| SEQ_ID_NO_10604 | | Useful for making plants with |
| | | modulated flowering time |
| SEQ_ID_NO_10604 | The plant is abnormally small. | Useful for making plants with |
| | | modulated biomass |
| SEQ_ID_NO_10604 | | Useful for making plants with |
| | | modulated biomass |
| SEQ_ID_NO_10616 | | Useful for making plants with |
| | | modulated nitrogen use efficiency |
| SEQ_ID_NO_10630 | The plant senesces significantly late | Useful for making plants with |
| | copared to wild-type. | modulated time to senescence |
| SEQ_ID_NO_10630 | The plant flowers significantly late | Useful for making plants with |
| | compared to wild-type. | modulated flowering time |
| SEQ_ID_NO_10632 | The plant is abnormally small. | Useful for making plants with |
| | | modulated biomass |
| SEQ_ID_NO_10632 | The plant produces a reduced number | Useful for biocontainment applications |
| | of seeds. | |
| SEQ_ID_NO_10686 | Useful for making plants with | The sequence is a functional homolog |
| | modulated growth and phenotype | of SEQ ID NO 654 in the 11/435,388 |
| | characteristics | US patent application |
| SEQ_ID_NO_10686 | Useful for making plants with | The sequence is a functional homolog |
| | modulated growth and phenotype | of SEQ ID NO 292 in the 10/225,066 |
| | characteristics | US patent application |
| SEQ_ID_NO_10686 | Useful for making plants with | The sequence is a functional homolog |
| | modulated growth and phenotype | of SEQ ID NO 2104 in the 10/374,780 |
| | characteristics | US patent application |
| SEQ_ID_NO_10686 | Useful for making plants with | The sequence is a functional homolog |
| | modulated growth and phenotype | of SEQ ID NO 206 in the 11/375,241 |
| | characteristics | US patent application |
| SEQ_ID_NO_10686 | Useful for making plants with | The sequence is a functional homolog |
| | modulated growth and phenotype | of SEQ ID NO 1858 in the 10/412,699 |
| | characteristics | US patent application |
| SEQ_ID_NO_10686 | Useful for making plants with | The sequence is a functional homolog |
| | modulated growth and phenotype | of SEQ ID NO 16 in the 10/412,699 |
| | characteristics | US patent application |
| SEQ_ID_NO_10686 | Useful for making plants with | The sequence is a functional homolog |
| | modulated growth and phenotype | of SEQ ID NO 1442 in the 10/666,642 |
| | characteristics | US patent application |
| SEQ_ID_NO_10700 | | Useful for making plants with altered |
| | | Lignin content |
| SEQ_ID_NO_10700 | | Useful for making plants with altered |
| | | alkaloid content |
| SEQ_ID_NO_10708 | | Useful for making plants with |
| | | modulated nitrogen use efficiency |
| SEQ_ID_NO_10728 | The plant flowers significantly late | Useful for making plants with |
| | compared to wild-type. | modulated flowering time |
| SEQ_ID_NO_10728 | The plant is abnormally small. | Useful for making plants with |
| | | modulated biomass |
| SEQ_ID_NO_10806 | Useful for making plants with | The sequence is a functional homolog |
| | modulated growth and phenotype | of SEQ ID NO 1248 in the 10/374,780 |
| | characteristics | US patent application |
| SEQ_ID_NO_10824 | Useful for making plants with | The sequence is a functional homolog |
| | modulated growth and phenotype | of SEQ ID NO 793 in the 11/479,226 |
| | characteristics | US patent application |
| SEQ_ID_NO_1094 | | Useful for making plants with |
| | | modulated heat sensitivity |
| SEQ_ID_NO_1094 | | Useful for making plants with altered |
| | | Lignin content |
| SEQ_ID_NO_10988 | The plant produces a reduced number | Useful for biocontainment applications |
| | of seeds. | |
| SEQ_ID_NO_11020 | Useful for making plants with | The sequence is a functional homolog |
| | modulated growth and phenotype | of SEQ ID NO 1701 in the 10/412,699 |
| | characteristics | US patent application |
| SEQ_ID_NO_11046 | | Useful for making plants with altered |
| | | cell wall content and/or composition |
| SEQ_ID_NO_1120 | Useful for making plants with | The sequence is a functional homolog |
| | modulated growth and phenotype | of SEQ ID NO 851 in the 10/666,642 |
| | characteristics | US patent application |
| SEQ_ID_NO_1120 | Useful for making plants with | The sequence is a functional homolog |
| | modulated growth and phenotype | of SEQ ID NO 1041 in the 10/374,780 |
| | characteristics | US patent application |
| SEQ_ID_NO_1136 | | Useful for making plants with |
| | | modulated nitrogen use efficiency |
| SEQ_ID_NO_1192 | | Useful for making plants with altered |
| | | alkaloid content |
| SEQ_ID_NO_1228 | The plant flowers significantly earllier | Useful for making plants with |
| | than wild-type. | modulated flowering time |
| SEQ_ID_NO_1228 | The plant is abnormally small. | Useful for making plants with |
| | | modulated biomass |
| SEQ_ID_NO_1228 | The plant produces a reduced number | Useful for biocontainment applications |
| | of seeds. | |
| SEQ_ID_NO_1350 | | Useful for making plants with |
| | | modulated drought sensitivity |
| SEQ_ID_NO_1356 | | Useful for making plants with altered |
| | | Lignin content |
| SEQ_ID_NO_1356 | | Useful for making plants with altered |
| | | cell wall content and/or composition |
| SEQ_ID_NO_1356 | | Useful for making plants with altered |
| | | cell wall composition |
| SEQ_ID_NO_1370 | | Useful for making plants with altered |
| | | terpenoid content |
| SEQ_ID_NO_1386 | | Useful for making plants with |
| | | modulated low light sensitivity |
| SEQ_ID_NO_1386 | The plant flowers significantly late | Useful for making plants with |
| | compared to wild-type. | modulated flowering time |
| SEQ_ID_NO_1432 | | Useful for making plants with altered |
| | | cell wall content and/or composition |
| SEQ_ID_NO_1440 | | Useful for making plants with altered |
| | | cell wall content and/or composition |
| SEQ_ID_NO_1442 | | Useful for making plants with altered |
| | | Lignin content |
| SEQ_ID_NO_1564 | Useful for making plants with | The sequence is a functional homolog |
| | modulated growth and phenotype | of SEQ ID NO 738 in the 10/374,780 |
| | characteristics | US patent application |
| SEQ_ID_NO_1592 | | Useful for making plants with altered |
| | | cell wall content and/or composition |
| SEQ_ID_NO_1640 | The plant flowers significantly earllier | Useful for making plants with |
| | than wild-type. | modulated flowering time |
| SEQ_ID_NO_1640 | The plant is abnormally small. | Useful for making plants with |
| | | modulated biomass |
| SEQ_ID_NO_1640 | | Useful for making plants with |
| | | modulated biomass |
| SEQ_ID_NO_1640 | Useful for making plants with | The sequence is a functional homolog |
| | modulated growth and phenotype | of SEQ ID NO 30 in the 10/666,642 |
| | characteristics | US patent application |
| SEQ_ID_NO_1640 | Useful for making plants with | The sequence is a functional homolog |
| | modulated growth and phenotype | of SEQ ID NO 26 in the 10/666,642 |
| | characteristics | US patent application |
| SEQ_ID_NO_1642 | | Useful for making plants with |
| | | modulated flowering time |
| SEQ_ID_NO_1654 | Useful for making plants with | The sequence is a functional homolog |
| | modulated growth and phenotype | of SEQ ID NO 420 in the 10/666,642 |
| | characteristics | US patent application |
| SEQ_ID_NO_1654 | Useful for making plants with | The sequence is a functional homolog |
| | modulated growth and phenotype | of SEQ ID NO 1506 in the 10/666,642 |
| | characteristics | US patent application |
| SEQ_ID_NO_1670 | The plant flowers significantly late | Useful for making plants with |
| | compared to wild-type. | modulated flowering time |
| SEQ_ID_NO_1670 | The plant produces a reduced number | Useful for biocontainment applications |
| | of seeds. | |
| SEQ_ID_NO_1682 | Useful for making plants with | The sequence is a functional homolog |
| | modulated growth and phenotype | of SEQ ID NO 876 in the 10/412,699 |
| | characteristics | US patent application |
| SEQ_ID_NO_1682 | Useful for making plants with | The sequence is a functional homolog |
| | modulated growth and phenotype | of SEQ ID NO 716 in the 10/412,699 |
| | characteristics | US patent application |
| SEQ_ID_NO_1682 | Useful for making plants with | The sequence is a functional homolog |
| | modulated growth and phenotype | of SEQ ID NO 644 in the 11/435,388 |
| | characteristics | US patent application |
| SEQ_ID_NO_1682 | Useful for making plants with | The sequence is a functional homolog |
| | modulated growth and phenotype | of SEQ ID NO 438 in the 10/666,642 |
| | characteristics | US patent application |
| SEQ_ID_NO_1682 | Useful for making plants with | The sequence is a functional homolog |
| | modulated growth and phenotype | of SEQ ID NO 20 in the 10/286,264 |
| | characteristics | US patent application |
| SEQ_ID_NO_1682 | Useful for making plants with | The sequence is a functional homolog |
| | modulated growth and phenotype | of SEQ ID NO 1773 in the 11/479,226 |
| | characteristics | US patent application |
| SEQ_ID_NO_1682 | Useful for making plants with | The sequence is a functional homolog |
| | modulated growth and phenotype | of SEQ ID NO 1492 in the 11/479,226 |
| | characteristics | US patent application |
| SEQ_ID_NO_1702 | | Useful for making plants with |
| | | modulated biomass |
| SEQ_ID_NO_1704 | | Useful for making plants with altered |
| | | cell wall content and/or composition |
| SEQ_ID_NO_172 | The plant flowers significantly late | Useful for making plants with |
| | compared to wild-type. | modulated flowering time |
| SEQ_ID_NO_172 | The plant is abnormally small. | Useful for making plants with |
| | | modulated biomass |
| SEQ_ID_NO_1720 | The plant flowers significantly earllier | Useful for making plants with |
| | than wild-type. | modulated flowering time |
| SEQ_ID_NO_1720 | The plant is abnormally small. | Useful for making plants with |
| | | modulated biomass |
| SEQ_ID_NO_1720 | | Useful for making plants with |
| | | modulated biomass |
| SEQ_ID_NO_1720 | Useful for making plants with | The sequence is a functional homolog |
| | modulated growth and phenotype | of SEQ ID NO 30 in the 10/666,642 |
| | characteristics | US patent application |
| SEQ_ID_NO_1720 | Useful for making plants with | The sequence is a functional homolog |
| | modulated growth and phenotype | of SEQ ID NO 26 in the 10/666,642 |
| | characteristics | US patent application |
| SEQ_ID_NO_1722 | The plant flowers significantly late | Useful for making plants with |
| | compared to wild-type. | modulated flowering time |
| SEQ_ID_NO_1722 | | Useful for making plants with |
| | | modulated cold sensitivity |
| SEQ_ID_NO_1742 | The plant flowers significantly late | Useful for making plants with |
| | compared to wild-type. | modulated flowering time |
| SEQ_ID_NO_1760 | The plant flowers significantly late | Useful for making plants with |
| | compared to wild-type. | modulated flowering time |
| SEQ_ID_NO_1760 | No seed is being produced by the plant | Useful for biocontainment applications |
| SEQ_ID_NO_1764 | Useful for making plants with | The sequence is a functional homolog |
| | modulated growth and phenotype | of SEQ ID NO 1839 in the 10/374,780 |
| | characteristics | US patent application |
| SEQ_ID_NO_1816 | | Useful for making plants with |
| | | modulated nitrogen use efficiency |
| SEQ_ID_NO_1820 | | Useful for making plants with altered |
| | | Lignin content |
| SEQ_ID_NO_1820 | | Useful for making plants with altered |
| | | cell wall content and/or composition |
| SEQ_ID_NO_1820 | | Useful for making plants with altered |
| | | cell wall composition |
| SEQ_ID_NO_1822 | Useful for making plants with | The sequence is a functional homolog |
| | modulated growth and phenotype | of SEQ ID NO 210 in the 10/666,642 |
| | characteristics | US patent application |
| SEQ_ID_NO_1826 | | Useful for making plants with altered |
| | | Lignin content |
| SEQ_ID_NO_1826 | | Useful for making plants with altered |
| | | alkaloid content |
| SEQ_ID_NO_1832 | | Useful for making plants with altered |
| | | Lignin content |
| SEQ_ID_NO_1832 | | Useful for making plants with altered |
| | | alkaloid content |
| SEQ_ID_NO_1860 | The plant flowers significantly earllier | Useful for making plants with |
| | than wild-type. | modulated flowering time |
| SEQ_ID_NO_1860 | | Useful for making plants with |
| | | modulated flowering time |
| SEQ_ID_NO_1860 | The plant is abnormally small. | Useful for making plants with |
| | | modulated biomass |
| SEQ_ID_NO_1860 | | Useful for making plants with |
| | | modulated biomass |
| SEQ_ID_NO_1862 | | Useful for making plants with |
| | | modulated cold sensitivity |
| SEQ_ID_NO_1920 | The plant flowers significantly late | Useful for making plants with |
| | compared to wild-type. | modulated flowering time |
| SEQ_ID_NO_1920 | No seed is being produced by the plant | Useful for biocontainment applications |
| SEQ_ID_NO_1922 | | Useful for making plants with altered |
| | | cell wall content and/or composition |
| SEQ_ID_NO_1926 | The plant flowers significantly late | Useful for making plants with |
| | compared to wild-type. | modulated flowering time |
| SEQ_ID_NO_1926 | No seed is being produced by the plant | Useful for biocontainment applications |
| SEQ_ID_NO_1934 | Useful for making plants with | The sequence is a functional homolog |
| | modulated growth and phenotype | of SEQ ID NO 501 in the 10/374,780 |
| | characteristics | US patent application |
| SEQ_ID_NO_1934 | Useful for making plants with | The sequence is a functional homolog |
| | modulated growth and phenotype | of SEQ ID NO 1000 in the 10/412,699 |
| | characteristics | US patent application |
| SEQ_ID_NO_1948 | | Useful for making plants with |
| | | modulated flowering time |
| SEQ_ID_NO_1950 | | Useful for making plants with altered |
| | | terpenoid content |
| SEQ_ID_NO_1950 | | Useful for making plants with altered |
| | | alkaloid content |
| SEQ_ID_NO_1954 | Useful for making plants with | The sequence is a functional homolog |
| | modulated growth and phenotype | of SEQ ID NO 712 in the 11/479,226 |
| | characteristics | US patent application |
| SEQ_ID_NO_1956 | The plant flowers significantly late | Useful for making plants with |
| | compared to wild-type. | modulated flowering time |
| SEQ_ID_NO_1958 | | Useful for making plants with altered |
| | | terpenoid content |
| SEQ_ID_NO_1958 | | Useful for making plants with altered |
| | | Lignin content |
| SEQ_ID_NO_1958 | Useful for making plants with | The sequence is a functional homolog |
| | modulated growth and phenotype | of SEQ ID NO 712 in the 10/374,780 |
| | characteristics | US patent application |
| SEQ_ID_NO_1958 | Useful for making plants with | The sequence is a functional homolog |
| | modulated growth and phenotype | of SEQ ID NO 709 in the 10/374,780 |
| | characteristics | US patent application |
| SEQ_ID_NO_1958 | Useful for making plants with | The sequence is a functional homolog |
| | modulated growth and phenotype | of SEQ ID NO 708 in the 10/374,780 |
| | characteristics | US patent application |
| SEQ_ID_NO_1958 | Useful for making plants with | The sequence is a functional homolog |
| | modulated growth and phenotype | of SEQ ID NO 686 in the 11/435,388 |
| | characteristics | US patent application |
| SEQ_ID_NO_1958 | Useful for making plants with | The sequence is a functional homolog |
| | modulated growth and phenotype | of SEQ ID NO 2188 in the 10/374,780 |
| | characteristics | US patent application |
| SEQ_ID_NO_196 | The plant is abnormally small. | Useful for making plants with |
| | | modulated biomass |
| SEQ_ID_NO_196 | The plant produces a reduced number | Useful for biocontainment applications |
| | of seeds. | |
| SEQ_ID_NO_1970 | | Useful for making plants with altered |
| | | cell wall content and/or composition |
| SEQ_ID_NO_1974 | | Useful for making plants with |
| | | modulated drought sensitivity |
| SEQ_ID_NO_1974 | Useful for making plants with | The sequence is a functional homolog |
| | modulated growth and phenotype | of SEQ ID NO 805 in the 11/479,226 |
| | characteristics | US patent application |
| SEQ_ID_NO_1974 | Useful for making plants with | The sequence is a functional homolog |
| | modulated growth and phenotype | of SEQ ID NO 801 in the 11/479,226 |
| | characteristics | US patent application |
| SEQ_ID_NO_1974 | Useful for making plants with | The sequence is a functional homolog |
| | modulated growth and phenotype | of SEQ ID NO 797 in the 11/479,226 |
| | characteristics | US patent application |
| SEQ_ID_NO_1974 | Useful for making plants with | The sequence is a functional homolog |
| | modulated growth and phenotype | of SEQ ID NO 790 in the 10/225,066 |
| | characteristics | US patent application |
| SEQ_ID_NO_1974 | Useful for making plants with | The sequence is a functional homolog |
| | modulated growth and phenotype | of SEQ ID NO 789 in the 11/479,226 |
| | characteristics | US patent application |
| SEQ_ID_NO_1974 | Useful for making plants with | The sequence is a functional homolog |
| | modulated growth and phenotype | of SEQ ID NO 786 in the 11/479,226 |
| | characteristics | US patent application |
| SEQ_ID_NO_1974 | Useful for making plants with | The sequence is a functional homolog |
| | modulated growth and phenotype | of SEQ ID NO 782 in the 11/479,226 |
| | characteristics | US patent application |
| SEQ_ID_NO_1974 | Useful for making plants with | The sequence is a functional homolog |
| | modulated growth and phenotype | of SEQ ID NO 344 in the 10/412,699 |
| | characteristics | US patent application |
| SEQ_ID_NO_1974 | Useful for making plants with | The sequence is a functional homolog |
| | modulated growth and phenotype | of SEQ ID NO 342 in the 10/412,699 |
| | characteristics | US patent application |
| SEQ_ID_NO_1974 | Useful for making plants with | The sequence is a functional homolog |
| | modulated growth and phenotype | of SEQ ID NO 2350 in the 10/374,780 |
| | characteristics | US patent application |
| SEQ_ID_NO_1974 | Useful for making plants with | The sequence is a functional homolog |
| | modulated growth and phenotype | of SEQ ID NO 1777 in the 11/479,226 |
| | characteristics | US patent application |
| SEQ_ID_NO_1994 | | Useful for making plants with |
| | | modulated nitrogen use efficiency |
| SEQ_ID_NO_1994 | The plant flowers significantly late | Useful for making plants with |
| | compared to wild-type. | modulated flowering time |
| SEQ_ID_NO_1994 | | Useful for making plants with altered |
| | | Lignin content |
| SEQ_ID_NO_2012 | The plant flowers significantly late | Useful for making plants with |
| | compared to wild-type. | modulated flowering time |
| SEQ_ID_NO_2012 | No seed is being produced by the plant | Useful for biocontainment applications |
| SEQ_ID_NO_202 | Useful for making plants with | The sequence is a functional homolog |
| | modulated growth and phenotype | of SEQ ID NO 186 in the 11/435,388 |
| | characteristics | US patent application |
| SEQ_ID_NO_202 | Useful for making plants with | The sequence is a functional homolog |
| | modulated growth and phenotype | of SEQ ID NO 186 in the 10/714,887 |
| | characteristics | US patent application |
| SEQ_ID_NO_202 | Useful for making plants with | The sequence is a functional homolog |
| | modulated growth and phenotype | of SEQ ID NO 178 in the 11/435,388 |
| | characteristics | US patent application |
| SEQ_ID_NO_202 | Useful for making plants with | The sequence is a functional homolog |
| | modulated growth and phenotype | of SEQ ID NO 178 in the 10/714,887 |
| | characteristics | US patent application |
| SEQ_ID_NO_202 | Useful for making plants with | The sequence is a functional homolog |
| | modulated growth and phenotype | of SEQ ID NO 174 in the 11/435,388 |
| | characteristics | US patent application |
| SEQ_ID_NO_202 | Useful for making plants with | The sequence is a functional homolog |
| | modulated growth and phenotype | of SEQ ID NO 174 in the 10/714,887 |
| | characteristics | US patent application |
| SEQ_ID_NO_2030 | The plant is abnormally small. | Useful for making plants with |
| | | modulated biomass |
| SEQ_ID_NO_2056 | The plant flowers significantly late | Useful for making plants with |
| | compared to wild-type. | modulated flowering time |
| SEQ_ID_NO_2056 | No seed is being produced by the plant | Useful for biocontainment applications |
| SEQ_ID_NO_2086 | The plant flowers significantly late | Useful for making plants with |
| | compared to wild-type. | modulated flowering time |
| SEQ_ID_NO_2090 | | Useful for making plants with |
| | | modulated oxidative stress sensitivity |
| SEQ_ID_NO_2094 | Useful for making plants with | The sequence is a functional homolog |
| | modulated growth and phenotype | of SEQ ID NO 462 in the 10/412,699 |
| | characteristics | US patent application |
| SEQ_ID_NO_2094 | Useful for making plants with | The sequence is a functional homolog |
| | modulated growth and phenotype | of SEQ ID NO 1064 in the 11/479,226 |
| | characteristics | US patent application |
| SEQ_ID_NO_2102 | Useful for making plants with | The sequence is a functional homolog |
| | modulated growth and phenotype | of SEQ ID NO 712 in the 11/479,226 |
| | characteristics | US patent application |
| SEQ_ID_NO_2102 | Useful for making plants with | The sequence is a functional homolog |
| | modulated growth and phenotype | of SEQ ID NO 1109 in the 10/374,780 |
| | characteristics | US patent application |
| SEQ_ID_NO_2108 | | Useful for making plants with altered |
| | | cell wall content and/or composition |
| SEQ_ID_NO_2112 | | Useful for making plants with altered |
| | | cell wall content and/or composition |
| SEQ_ID_NO_2132 | The plant senesces significantly late | Useful for making plants with |
| | copared to wild-type. | modulated time to senescence |
| SEQ_ID_NO_2132 | The plant is abnormally small. | Useful for making plants with |
| | | modulated biomass |
| SEQ_ID_NO_2132 | No seed is being produced by the plant | Useful for biocontainment applications |
| SEQ_ID_NO_2180 | | Useful for making plants with |
| | | modulated nitrogen use efficiency |
| SEQ_ID_NO_2180 | | Useful for making plants with altered |
| | | cell wall content and/or composition |
| SEQ_ID_NO_2180 | Useful for making plants with | The sequence is a functional homolog |
| | modulated growth and phenotype | of SEQ ID NO 460 in the 10/412,699 |
| | characteristics | US patent application |
| SEQ_ID_NO_2180 | Useful for making plants with | The sequence is a functional homolog |
| | modulated growth and phenotype | of SEQ ID NO 386 in the 10/374,780 |
| | characteristics | US patent application |
| SEQ_ID_NO_2180 | Useful for making plants with | The sequence is a functional homolog |
| | modulated growth and phenotype | of SEQ ID NO 1094 in the 10/225,066 |
| | characteristics | US patent application |
| SEQ_ID_NO_2180 | Useful for making plants with | The sequence is a functional homolog |
| | modulated growth and phenotype | of SEQ ID NO 1060 in the 11/479,226 |
| | characteristics | US patent application |
| SEQ_ID_NO_2186 | | Useful for making plants with altered |
| | | Lignin content |
| SEQ_ID_NO_22 | The plant flowers significantly late | Useful for making plants with |
| | compared to wild-type. | modulated flowering time |
| SEQ_ID_NO_22 | No seed is being produced by the plant | Useful for biocontainment applications |
| SEQ_ID_NO_2228 | | Useful for making plants with altered |
| | | cell wall content and/or composition |
| SEQ_ID_NO_2238 | The plant senesces significantly late | Useful for making plants with |
| | copared to wild-type. | modulated time to senescence |
| SEQ_ID_NO_2252 | The plant flowers significantly late | Useful for making plants with |
| | compared to wild-type. | modulated flowering time |
| SEQ_ID_NO_2252 | No seed is being produced by the plant | Useful for biocontainment applications |
| SEQ_ID_NO_2260 | | Useful for making plants with altered |
| | | terpenoid content |
| SEQ_ID_NO_2260 | | Useful for making plants with altered |
| | | Lignin content |
| SEQ_ID_NO_2260 | | Useful for making plants with altered |
| | | alkaloid content |
| SEQ_ID_NO_2282 | The plant flowers significantly late | Useful for making plants with |
| | compared to wild-type. | modulated flowering time |
| SEQ_ID_NO_2282 | No seed is being produced by the plant | Useful for biocontainment applications |
| SEQ_ID_NO_2294 | The plant flowers significantly late | Useful for making plants with |
| | compared to wild-type. | modulated flowering time |
| SEQ_ID_NO_2294 | No seed is being produced by the plant | Useful for biocontainment applications |
| SEQ_ID_NO_2298 | The plant produces a reduced number | Useful for biocontainment applications |
| | of seeds. | |
| SEQ_ID_NO_2308 | The plant flowers significantly late | Useful for making plants with |
| | compared to wild-type. | modulated flowering time |
| SEQ_ID_NO_2308 | No seed is being produced by the plant | Useful for biocontainment applications |
| SEQ_ID_NO_2330 | | Useful for making plants with |
| | | modulated phosphate use efficiency |
| SEQ_ID_NO_2330 | | Useful for making plants with |
| | | modulated nitrogen use efficiency |
| SEQ_ID_NO_2354 | The plant flowers significantly late | Useful for making plants with |
| | compared to wild-type. | modulated flowering time |
| SEQ_ID_NO_2354 | No seed is being produced by the plant | Useful for biocontainment applications |
| SEQ_ID_NO_2362 | | Useful for making plants with |
| | | modulated nitrogen use efficiency |
| SEQ_ID_NO_2362 | The plant flowers significantly late | Useful for making plants with |
| | compared to wild-type. | modulated flowering time |
| SEQ_ID_NO_2362 | | Useful for making plants with altered |
| | | Lignin content |
| SEQ_ID_NO_2362 | Useful for making plants with | The sequence is a functional homolog |
| | modulated growth and phenotype | of SEQ ID NO 1016 in the 11/435,388 |
| | characteristics | US patent application |
| SEQ_ID_NO_2382 | | Useful for making plants with altered |
| | | alkaloid content |
| SEQ_ID_NO_2392 | | Useful for making plants with |
| | | modulated low light sensitivity |
| SEQ_ID_NO_2392 | The plant flowers significantly late | Useful for making plants with |
| | compared to wild-type. | modulated flowering time |
| SEQ_ID_NO_2424 | | Useful for making plants with |
| | | modulated phosphate use efficiency |
| SEQ_ID_NO_2424 | The plant flowers significantly late | Useful for making plants with |
| | compared to wild-type. | modulated flowering time |
| SEQ_ID_NO_2424 | The plant is abnormally large. | Useful for making plants with |
| | | modulated biomass |
| SEQ_ID_NO_244 | The plant flowers significantly late | Useful for making plants with |
| | compared to wild-type. | modulated flowering time |
| SEQ_ID_NO_244 | No seed is being produced by the plant | Useful for biocontainment applications |
| SEQ_ID_NO_2440 | | Useful for making plants with altered |
| | | Lignin content |
| SEQ_ID_NO_2440 | | Useful for making plants with altered |
| | | alkaloid content |
| SEQ_ID_NO_2488 | | Useful for making plants with |
| | | modulated cold sensitivity |
| SEQ_ID_NO_2540 | Useful for making plants with | The sequence is a functional homolog |
| | modulated growth and phenotype | of SEQ ID NO 92 in the 10/669,824 |
| | characteristics | US patent application |
| SEQ_ID_NO_2540 | Useful for making plants with | The sequence is a functional homolog |
| | modulated growth and phenotype | of SEQ ID NO 88 in the 10/669,824 |
| | characteristics | US patent application |
| SEQ_ID_NO_2540 | Useful for making plants with | The sequence is a functional homolog |
| | modulated growth and phenotype | of SEQ ID NO 48 in the 10/870,198 |
| | characteristics | US patent application |
| SEQ_ID_NO_2540 | Useful for making plants with | The sequence is a functional homolog |
| | modulated growth and phenotype | of SEQ ID NO 48 in the 10/669,824 |
| | characteristics | US patent application |
| SEQ_ID_NO_2540 | Useful for making plants with | The sequence is a functional homolog |
| | modulated growth and phenotype | of SEQ ID NO 392 in the 10/714,887 |
| | characteristics | US patent application |
| SEQ_ID_NO_2540 | Useful for making plants with | The sequence is a functional homolog |
| | modulated growth and phenotype | of SEQ ID NO 390 in the 11/435,388 |
| | characteristics | US patent application |
| SEQ_ID_NO_2540 | Useful for making plants with | The sequence is a functional homolog |
| | modulated growth and phenotype | of SEQ ID NO 390 in the 10/714,887 |
| | characteristics | US patent application |
| SEQ_ID_NO_2540 | Useful for making plants with | The sequence is a functional homolog |
| | modulated growth and phenotype | of SEQ ID NO 388 in the 11/435,388 |
| | characteristics | US patent application |
| SEQ_ID_NO_2540 | Useful for making plants with | The sequence is a functional homolog |
| | modulated growth and phenotype | of SEQ ID NO 388 in the 10/714,887 |
| | characteristics | US patent application |
| SEQ_ID_NO_2540 | Useful for making plants with | The sequence is a functional homolog |
| | modulated growth and phenotype | of SEQ ID NO 386 in the 11/435,388 |
| | characteristics | US patent application |
| SEQ_ID_NO_2540 | Useful for making plants with | The sequence is a functional homolog |
| | modulated growth and phenotype | of SEQ ID NO 300 in the 10/714,887 |
| | characteristics | US patent application |
| SEQ_ID_NO_2540 | Useful for making plants with | The sequence is a functional homolog |
| | modulated growth and phenotype | of SEQ ID NO 298 in the 11/435,388 |
| | characteristics | US patent application |
| SEQ_ID_NO_2540 | Useful for making plants with | The sequence is a functional homolog |
| | modulated growth and phenotype | of SEQ ID NO 256 in the 10/225,066 |
| | characteristics | US patent application |
| SEQ_ID_NO_2540 | Useful for making plants with | The sequence is a functional homolog |
| | modulated growth and phenotype | of SEQ ID NO 2528 in the 10/374,780 |
| | characteristics | US patent application |
| SEQ_ID_NO_2540 | Useful for making plants with | The sequence is a functional homolog |
| | modulated growth and phenotype | of SEQ ID NO 24 in the 10/870,198 |
| | characteristics | US patent application |
| SEQ_ID_NO_2540 | Useful for making plants with | The sequence is a functional homolog |
| | modulated growth and phenotype | of SEQ ID NO 24 in the 10/669,824 |
| | characteristics | US patent application |
| SEQ_ID_NO_2540 | Useful for making plants with | The sequence is a functional homolog |
| | modulated growth and phenotype | of SEQ ID NO 18 in the 10/870,198 |
| | characteristics | US patent application |
| SEQ_ID_NO_2540 | Useful for making plants with | The sequence is a functional homolog |
| | modulated growth and phenotype | of SEQ ID NO 18 in the 10/669,824 |
| | characteristics | US patent application |
| SEQ_ID_NO_2540 | Useful for making plants with | The sequence is a functional homolog |
| | modulated growth and phenotype | of SEQ ID NO 16 in the 10/870,198 |
| | characteristics | US patent application |
| SEQ_ID_NO_2540 | Useful for making plants with | The sequence is a functional homolog |
| | modulated growth and phenotype | of SEQ ID NO 16 in the 10/669,824 |
| | characteristics | US patent application |
| SEQ_ID_NO_2540 | Useful for making plants with | The sequence is a functional homolog |
| | modulated growth and phenotype | of SEQ ID NO 1417 in the 10/374,780 |
| | characteristics | US patent application |
| SEQ_ID_NO_2540 | Useful for making plants with | The sequence is a functional homolog |
| | modulated growth and phenotype | of SEQ ID NO 1416 in the 10/374,780 |
| | characteristics | US patent application |
| SEQ_ID_NO_2540 | Useful for making plants with | The sequence is a functional homolog |
| | modulated growth and phenotype | of SEQ ID NO 1415 in the 10/374,780 |
| | characteristics | US patent application |
| SEQ_ID_NO_2540 | Useful for making plants with | The sequence is a functional homolog |
| | modulated growth and phenotype | of SEQ ID NO 1414 in the 10/374,780 |
| | characteristics | US patent application |
| SEQ_ID_NO_2578 | The plant flowers significantly late | Useful for making plants with |
| | compared to wild-type. | modulated flowering time |
| SEQ_ID_NO_2578 | No seed is being produced by the plant | Useful for biocontainment applications |
| SEQ_ID_NO_2588 | | Useful for making plants with altered |
| | | terpenoid content |
| SEQ_ID_NO_2588 | | Useful for making plants with altered |
| | | alkaloid content |
| SEQ_ID_NO_2598 | | Useful for making plants with altered |
| | | cell wall content and/or composition |
| SEQ_ID_NO_2666 | The plant flowers significantly earllier | Useful for making plants with |
| | than wild-type. | modulated flowering time |
| SEQ_ID_NO_2666 | | Useful for making plants with |
| | | modulated flowering time |
| SEQ_ID_NO_2666 | The plant is abnormally small. | Useful for making plants with |
| | | modulated biomass |
| SEQ_ID_NO_2666 | | Useful for making plants with |
| | | modulated biomass |
| SEQ_ID_NO_2718 | Useful for making plants with | The sequence is a functional homolog |
| | modulated growth and phenotype | of SEQ ID NO 805 in the 11/479,226 |
| | characteristics | US patent application |
| SEQ_ID_NO_2718 | Useful for making plants with | The sequence is a functional homolog |
| | modulated growth and phenotype | of SEQ ID NO 793 in the 11/479,226 |
| | characteristics | US patent application |
| SEQ_ID_NO_2718 | Useful for making plants with | The sequence is a functional homolog |
| | modulated growth and phenotype | of SEQ ID NO 790 in the 10/225,066 |
| | characteristics | US patent application |
| SEQ_ID_NO_2718 | Useful for making plants with | The sequence is a functional homolog |
| | modulated growth and phenotype | of SEQ ID NO 344 in the 10/412,699 |
| | characteristics | US patent application |
| SEQ_ID_NO_2718 | Useful for making plants with | The sequence is a functional homolog |
| | modulated growth and phenotype | of SEQ ID NO 2350 in the 10/374,780 |
| | characteristics | US patent application |
| SEQ_ID_NO_2722 | The plant produces a reduced number | Useful for biocontainment applications |
| | of seeds. | |
| SEQ_ID_NO_2752 | | Useful for making plants with |
| | | modulated salt sensitivity |
| SEQ_ID_NO_278 | The plant flowers significantly late | Useful for making plants with |
| | compared to wild-type. | modulated flowering time |
| SEQ_ID_NO_278 | No seed is being produced by the plant | Useful for biocontainment applications |
| SEQ_ID_NO_288 | The plant is abnormally small. | Useful for making plants with |
| | | modulated biomass |
| SEQ_ID_NO_2880 | | Useful for making plants with altered |
| | | Lignin content |
| SEQ_ID_NO_2898 | | Useful for making plants with |
| | | modulated phosphate use efficiency |
| SEQ_ID_NO_2898 | The plant flowers significantly late | Useful for making plants with |
| | compared to wild-type. | modulated flowering time |
| SEQ_ID_NO_2898 | The plant is abnormally large. | Useful for making plants with |
| | | modulated biomass |
| SEQ_ID_NO_2916 | | Useful for making plants with |
| | | modulated salt sensitivity |
| SEQ_ID_NO_2920 | The plant flowers significantly late | Useful for making plants with |
| | compared to wild-type. | modulated flowering time |
| SEQ_ID_NO_2920 | No seed is being produced by the plant | Useful for biocontainment applications |
| SEQ_ID_NO_294 | | Useful for making plants with |
| | | modulated cold sensitivity |
| SEQ_ID_NO_2940 | | Useful for making plants with |
| | | modulated phosphate use efficiency |
| SEQ_ID_NO_2940 | | Useful for making plants with |
| | | modulated nitrogen use efficiency |
| SEQ_ID_NO_2944 | The plant produces a reduced number | Useful for biocontainment applications |
| | of seeds. | |
| SEQ_ID_NO_2976 | The plant flowers significantly late | Useful for making plants with |
| | compared to wild-type. | modulated flowering time |
| SEQ_ID_NO_2976 | No seed is being produced by the plant | Useful for biocontainment applications |
| SEQ_ID_NO_2980 | | Useful for making plants with altered |
| | | Lignin content |
| SEQ_ID_NO_2980 | | Useful for making plants with altered |
| | | cell wall content and/or composition |
| SEQ_ID_NO_2980 | | Useful for making plants with altered |
| | | cell wall composition |
| SEQ_ID_NO_2988 | | Useful for making plants with |
| | | modulated flowering time |
| SEQ_ID_NO_2996 | | Useful for making plants with |
| | | modulated oxidative stress sensitivity |
| SEQ_ID_NO_300 | The plant is abnormally large. | Useful for making plants with |
| | | modulated biomass |
| SEQ_ID_NO_300 | The plant is abnormally small. | Useful for making plants with |
| | | modulated biomass |
| SEQ_ID_NO_3006 | | Useful for making plants with altered |
| | | Lignin content |
| SEQ_ID_NO_3006 | | Useful for making plants with altered |
| | | alkaloid content |
| SEQ_ID_NO_3012 | | Useful for making plants with |
| | | modulated flowering time |
| SEQ_ID_NO_3024 | | Useful for making plants with altered |
| | | cell wall content and/or composition |
| SEQ_ID_NO_3070 | | Useful for making plants with |
| | | modulated nitrogen use efficiency |
| SEQ_ID_NO_3080 | The plant flowers significantly late | Useful for making plants with |
| | compared to wild-type. | modulated flowering time |
| SEQ_ID_NO_3080 | The plant is abnormally small. | Useful for making plants with |
| | | modulated biomass |
| SEQ_ID_NO_3102 | | Useful for making plants with altered |
| | | terpenoid content |
| SEQ_ID_NO_3152 | The plant is abnormally small. | Useful for making plants with |
| | | modulated biomass |
| SEQ_ID_NO_3152 | The plant produces a reduced number | Useful for biocontainment applications |
| | of seeds. | |
| SEQ_ID_NO_3170 | The plant is abnormally large. | Useful for making plants with |
| | | modulated biomass |
| SEQ_ID_NO_3170 | Useful for making plants with | The sequence is a functional homolog |
| | modulated growth and phenotype | of SEQ ID NO 786 in the 10/412,699 |
| | characteristics | US patent application |
| SEQ_ID_NO_3170 | Useful for making plants with | The sequence is a functional homolog |
| | modulated growth and phenotype | of SEQ ID NO 784 in the 10/412,699 |
| | characteristics | US patent application |
| SEQ_ID_NO_3170 | Useful for making plants with | The sequence is a functional homolog |
| | modulated growth and phenotype | of SEQ ID NO 1617 in the 11/479,226 |
| | characteristics | US patent application |
| SEQ_ID_NO_3170 | Useful for making plants with | The sequence is a functional homolog |
| | modulated growth and phenotype | of SEQ ID NO 1614 in the 11/479,226 |
| | characteristics | US patent application |
| SEQ_ID_NO_3218 | | Useful for making plants with |
| | | modulated nitrogen use efficiency |
| SEQ_ID_NO_3282 | The plant flowers significantly late | Useful for making plants with |
| | compared to wild-type. | modulated flowering time |
| SEQ_ID_NO_3282 | No seed is being produced by the plant | Useful for biocontainment applications |
| SEQ_ID_NO_3306 | The plant flowers significantly late | Useful for making plants with |
| | compared to wild-type. | modulated flowering time |
| SEQ_ID_NO_3306 | No seed is being produced by the plant | Useful for biocontainment applications |
| SEQ_ID_NO_3316 | The plant is abnormally large. | Useful for making plants with |
| | | modulated biomass |
| SEQ_ID_NO_3316 | Useful for making plants with | The sequence is a functional homolog |
| | modulated growth and phenotype | of SEQ ID NO 786 in the 10/412,699 |
| | characteristics | US patent application |
| SEQ_ID_NO_3316 | Useful for making plants with | The sequence is a functional homolog |
| | modulated growth and phenotype | of SEQ ID NO 784 in the 10/412,699 |
| | characteristics | US patent application |
| SEQ_ID_NO_3316 | Useful for making plants with | The sequence is a functional homolog |
| | modulated growth and phenotype | of SEQ ID NO 1617 in the 11/479,226 |
| | characteristics | US patent application |
| SEQ_ID_NO_3316 | Useful for making plants with | The sequence is a functional homolog |
| | modulated growth and phenotype | of SEQ ID NO 1614 in the 11/479,226 |
| | characteristics | US patent application |
| SEQ_ID_NO_3352 | The plant produces a reduced number | Useful for biocontainment applications |
| | of seeds. | |
| SEQ_ID_NO_3364 | Useful for making plants with | The sequence is a functional homolog |
| | modulated growth and phenotype | of SEQ ID NO 739 in the 10/374,780 |
| | characteristics | US patent application |
| SEQ_ID_NO_3372 | The plant is abnormally large. | Useful for making plants with |
| | | modulated biomass |
| SEQ_ID_NO_3372 | Useful for making plants with | The sequence is a functional homolog |
| | modulated growth and phenotype | of SEQ ID NO 786 in the 10/412,699 |
| | characteristics | US patent application |
| SEQ_ID_NO_3372 | Useful for making plants with | The sequence is a functional homolog |
| | modulated growth and phenotype | of SEQ ID NO 784 in the 10/412,699 |
| | characteristics | US patent application |
| SEQ_ID_NO_3372 | Useful for making plants with | The sequence is a functional homolog |
| | modulated growth and phenotype | of SEQ ID NO 1617 in the 11/479,226 |
| | characteristics | US patent application |
| SEQ_ID_NO_3372 | Useful for making plants with | The sequence is a functional homolog |
| | modulated growth and phenotype | of SEQ ID NO 1614 in the 11/479,226 |
| | characteristics | US patent application |
| SEQ_ID_NO_3392 | | Useful for making plants with |
| | | modulated cold sensitivity |
| SEQ_ID_NO_3400 | | Useful for making plants with altered |
| | | cell wall content and/or composition |
| SEQ_ID_NO_3412 | | Useful for making plants with |
| | | modulated cold sensitivity |
| SEQ_ID_NO_3456 | The plant flowers significantly late | Useful for making plants with |
| | compared to wild-type. | modulated flowering time |
| SEQ_ID_NO_3456 | No seed is being produced by the plant | Useful for biocontainment applications |
| SEQ_ID_NO_3462 | | Useful for making plants with |
| | | modulated phosphate use efficiency |
| SEQ_ID_NO_3484 | | Useful for making plants with |
| | | modulated phosphate use efficiency |
| SEQ_ID_NO_3484 | The plant flowers significantly late | Useful for making plants with |
| | compared to wild-type. | modulated flowering time |
| SEQ_ID_NO_3484 | The plant is abnormally large. | Useful for making plants with |
| | | modulated biomass |
| SEQ_ID_NO_3518 | | Useful for making plants with altered |
| | | Lignin content |
| SEQ_ID_NO_3522 | The plant produces a reduced number | Useful for biocontainment applications |
| | of seeds. | |
| SEQ_ID_NO_3590 | | Useful for making plants with |
| | | modulated cold sensitivity |
| SEQ_ID_NO_3596 | The plant flowers significantly late | Useful for making plants with |
| | compared to wild-type. | modulated flowering time |
| SEQ_ID_NO_3596 | No seed is being produced by the plant | Useful for biocontainment applications |
| SEQ_ID_NO_3602 | | Useful for making plants with altered |
| | | terpenoid content |
| SEQ_ID_NO_3650 | | Useful for making plants with |
| | | modulated phosphate use efficiency |
| SEQ_ID_NO_3670 | | Useful for making plants with |
| | | modulated cold sensitivity |
| SEQ_ID_NO_3698 | Useful for making plants with | The sequence is a functional homolog |
| | modulated growth and phenotype | of SEQ ID NO 1503 in the 10/374,780 |
| | characteristics | US patent application |
| SEQ_ID_NO_3730 | The plant senesces significantly late | Useful for making plants with |
| | copared to wild-type. | modulated time to senescence |
| SEQ_ID_NO_3732 | | Useful for making plants with altered |
| | | Lignin content |
| SEQ_ID_NO_3746 | The plant flowers significantly late | Useful for making plants with |
| | compared to wild-type. | modulated flowering time |
| SEQ_ID_NO_3746 | No seed is being produced by the plant | Useful for biocontainment applications |
| SEQ_ID_NO_3752 | The plant flowers significantly late | Useful for making plants with |
| | compared to wild-type. | modulated flowering time |
| SEQ_ID_NO_3752 | No seed is being produced by the plant | Useful for biocontainment applications |
| SEQ_ID_NO_3820 | The plant flowers significantly late | Useful for making plants with |
| | compared to wild-type. | modulated flowering time |
| SEQ_ID_NO_3820 | No seed is being produced by the plant | Useful for biocontainment applications |
| SEQ_ID_NO_3852 | | Useful for making plants with altered |
| | | Lignin content |
| SEQ_ID_NO_386 | The plant is abnormally large. | Useful for making plants with |
| | | modulated biomass |
| SEQ_ID_NO_386 | Useful for making plants with | The sequence is a functional homolog |
| | modulated growth and phenotype | of SEQ ID NO 786 in the 10/412,699 |
| | characteristics | US patent application |
| SEQ_ID_NO_386 | Useful for making plants with | The sequence is a functional homolog |
| | modulated growth and phenotype | of SEQ ID NO 784 in the 10/412,699 |
| | characteristics | US patent application |
| SEQ_ID_NO_386 | Useful for making plants with | The sequence is a functional homolog |
| | modulated growth and phenotype | of SEQ ID NO 1617 in the 11/479,226 |
| | characteristics | US patent application |
| SEQ_ID_NO_386 | Useful for making plants with | The sequence is a functional homolog |
| | modulated growth and phenotype | of SEQ ID NO 1614 in the 11/479,226 |
| | characteristics | US patent application |
| SEQ_ID_NO_3860 | The plant is abnormally small. | Useful for making plants with |
| | | modulated biomass |
| SEQ_ID_NO_3872 | The plant flowers significantly late | Useful for making plants with |
| | compared to wild-type. | modulated flowering time |
| SEQ_ID_NO_3872 | No seed is being produced by the plant | Useful for biocontainment applications |
| SEQ_ID_NO_3876 | The plant is abnormally small. | Useful for making plants with |
| | | modulated biomass |
| SEQ_ID_NO_3878 | The plant flowers significantly late | Useful for making plants with |
| | compared to wild-type. | modulated flowering time |
| SEQ_ID_NO_3878 | No seed is being produced by the plant | Useful for biocontainment applications |
| SEQ_ID_NO_3982 | Useful for making plants with | The sequence is a functional homolog |
| | modulated growth and phenotype | of SEQ ID NO 126 in the 11/435,388 |
| | characteristics | US patent application |
| SEQ_ID_NO_3982 | Useful for making plants with | The sequence is a functional homolog |
| | modulated growth and phenotype | of SEQ ID NO 126 in the 10/714,887 |
| | characteristics | US patent application |
| SEQ_ID_NO_3986 | The plant flowers significantly late | Useful for making plants with |
| | compared to wild-type. | modulated flowering time |
| SEQ_ID_NO_3986 | The plant is abnormally small. | Useful for making plants with |
| | | modulated biomass |
| SEQ_ID_NO_4 | | Useful for making plants with |
| | | modulated nitrogen use efficiency |
| SEQ_ID_NO_4056 | The plant flowers significantly late | Useful for making plants with |
| | compared to wild-type. | modulated flowering time |
| SEQ_ID_NO_4056 | The plant is abnormally small. | Useful for making plants with |
| | | modulated biomass |
| SEQ_ID_NO_4126 | | Useful for making plants with altered |
| | | cell wall content and/or composition |
| SEQ_ID_NO_4142 | The plant flowers significantly late | Useful for making plants with |
| | compared to wild-type. | modulated flowering time |
| SEQ_ID_NO_4142 | No seed is being produced by the plant | Useful for biocontainment applications |
| SEQ_ID_NO_4164 | | Useful for making plants with |
| | | modulated phosphate use efficiency |
| SEQ_ID_NO_4164 | The plant flowers significantly late | Useful for making plants with |
| | compared to wild-type. | modulated flowering time |
| SEQ_ID_NO_4164 | The plant is abnormally large. | Useful for making plants with |
| | | modulated biomass |
| SEQ_ID_NO_4188 | The plant flowers significantly late | Useful for making plants with |
| | compared to wild-type. | modulated flowering time |
| SEQ_ID_NO_4188 | The plant is abnormally small. | Useful for making plants with |
| | | modulated biomass |
| SEQ_ID_NO_422 | | Useful for making plants with altered |
| | | Lignin content |
| SEQ_ID_NO_422 | | Useful for making plants with altered |
| | | alkaloid content |
| SEQ_ID_NO_4228 | The plant senesces significantly late | Useful for making plants with |
| | copared to wild-type. | modulated time to senescence |
| SEQ_ID_NO_4228 | The plant flowers significantly late | Useful for making plants with |
| | compared to wild-type. | modulated flowering time |
| SEQ_ID_NO_4238 | The plant flowers significantly late | Useful for making plants with |
| | compared to wild-type. | modulated flowering time |
| SEQ_ID_NO_4238 | No seed is being produced by the plant | Useful for biocontainment applications |
| SEQ_ID_NO_4292 | The plant produces a reduced number | Useful for biocontainment applications |
| | of seeds. | |
| SEQ_ID_NO_4314 | | Useful for making plants with altered |
| | | Lignin content |
| SEQ_ID_NO_4314 | | Useful for making plants with altered |
| | | alkaloid content |
| SEQ_ID_NO_4342 | The plant flowers significantly late | Useful for making plants with |
| | compared to wild-type. | modulated flowering time |
| SEQ_ID_NO_4342 | The plant is abnormally small. | Useful for making plants with |
| | | modulated biomass |
| SEQ_ID_NO_4364 | The plant flowers significantly late | Useful for making plants with |
| | compared to wild-type. | modulated flowering time |
| SEQ_ID_NO_4364 | No seed is being produced by the plant | Useful for biocontainment applications |
| SEQ_ID_NO_438 | | Useful for making plants with |
| | | modulated cold sensitivity |
| SEQ_ID_NO_4386 | The plant flowers significantly late | Useful for making plants with |
| | compared to wild-type. | modulated flowering time |
| SEQ_ID_NO_4386 | No seed is being produced by the plant | Useful for biocontainment applications |
| SEQ_ID_NO_4392 | | Useful for making plants with altered oil |
| | | content |
| SEQ_ID_NO_4410 | The plant is abnormally small. | Useful for making plants with |
| | | modulated biomass |
| SEQ_ID_NO_4418 | | Useful for making plants with |
| | | modulated heat sensitivity |
| SEQ_ID_NO_4418 | | Useful for making plants with altered |
| | | Lignin content |
| SEQ_ID_NO_4436 | The plant is abnormally small. | Useful for making plants with |
| | | modulated biomass |
| SEQ_ID_NO_4436 | The plant produces a reduced number | Useful for biocontainment applications |
| | of seeds. | |
| SEQ_ID_NO_4476 | | Useful for making plants with altered |
| | | cell wall content and/or composition |
| SEQ_ID_NO_4494 | | Useful for making plants with |
| | | modulated drought sensitivity |
| SEQ_ID_NO_4524 | | Useful for making plants with |
| | | modulated low light sensitivity |
| SEQ_ID_NO_4524 | The plant flowers significantly late | Useful for making plants with |
| | compared to wild-type. | modulated flowering time |
| SEQ_ID_NO_4592 | The plant is abnormally small. | Useful for making plants with |
| | | modulated biomass |
| SEQ_ID_NO_4592 | Useful for making plants with | The sequence is a functional homolog |
| | modulated growth and phenotype | of SEQ ID NO 960 in the 10/225,066 |
| | characteristics | US patent application |
| SEQ_ID_NO_4592 | Useful for making plants with | The sequence is a functional homolog |
| | modulated growth and phenotype | of SEQ ID NO 874 in the 10/412,699 |
| | characteristics | US patent application |
| SEQ_ID_NO_4592 | Useful for making plants with | The sequence is a functional homolog |
| | modulated growth and phenotype | of SEQ ID NO 2604 in the 10/374,780 |
| | characteristics | US patent application |
| SEQ_ID_NO_4592 | Useful for making plants with | The sequence is a functional homolog |
| | modulated growth and phenotype | of SEQ ID NO 1769 in the 11/479,226 |
| | characteristics | US patent application |
| SEQ_ID_NO_4592 | Useful for making plants with | The sequence is a functional homolog |
| | modulated growth and phenotype | of SEQ ID NO 1765 in the 11/479,226 |
| | characteristics | US patent application |
| SEQ_ID_NO_4592 | Useful for making plants with | The sequence is a functional homolog |
| | modulated growth and phenotype | of SEQ ID NO 1761 in the 11/479,226 |
| | characteristics | US patent application |
| SEQ_ID_NO_4604 | The plants are small. | Useful for making plants with |
| | | modulated biomass |
| SEQ_ID_NO_4604 | | Useful for making plants with |
| | | modulated biomass |
| SEQ_ID_NO_4610 | The plant is abnormally large. | Useful for making plants with |
| | | modulated biomass |
| SEQ_ID_NO_4610 | Useful for making plants with | The sequence is a functional homolog |
| | modulated growth and phenotype | of SEQ ID NO 786 in the 10/412,699 |
| | characteristics | US patent application |
| SEQ_ID_NO_4610 | Useful for making plants with | The sequence is a functional homolog |
| | modulated growth and phenotype | of SEQ ID NO 784 in the 10/412,699 |
| | characteristics | US patent application |
| SEQ_ID_NO_4610 | Useful for making plants with | The sequence is a functional homolog |
| | modulated growth and phenotype | of SEQ ID NO 1617 in the 11/479,226 |
| | characteristics | US patent application |
| SEQ_ID_NO_4610 | Useful for making plants with | The sequence is a functional homolog |
| | modulated growth and phenotype | of SEQ ID NO 1614 in the 11/479,226 |
| | characteristics | US patent application |
| SEQ_ID_NO_4666 | | Useful for making plants with altered |
| | | cell wall content and/or composition |
| SEQ_ID_NO_4720 | The plant is abnormally large. | Useful for making plants with |
| | | modulated biomass |
| SEQ_ID_NO_4748 | The plant flowers significantly late | Useful for making plants with |
| | compared to wild-type. | modulated flowering time |
| SEQ_ID_NO_4748 | No seed is being produced by the plant | Useful for biocontainment applications |
| SEQ_ID_NO_476 | | Useful for biocontainment applications |
| SEQ_ID_NO_4770 | The plant flowers significantly earllier | Useful for making plants with |
| | than wild-type. | modulated flowering time |
| SEQ_ID_NO_4770 | | Useful for making plants with |
| | | modulated flowering time |
| SEQ_ID_NO_4770 | The plant is abnormally small. | Useful for making plants with |
| | | modulated biomass |
| SEQ_ID_NO_4770 | | Useful for making plants with |
| | | modulated biomass |
| SEQ_ID_NO_48 | The plant flowers significantly late | Useful for making plants with |
| | compared to wild-type. | modulated flowering time |
| SEQ_ID_NO_48 | No seed is being produced by the plant | Useful for biocontainment applications |
| SEQ_ID_NO_4820 | Useful for making plants with | The sequence is a functional homolog |
| | modulated growth and phenotype | of SEQ ID NO 793 in the 11/479,226 |
| | characteristics | US patent application |
| SEQ_ID_NO_4878 | | Useful for making plants with |
| | | modulated phosphate use efficiency |
| SEQ_ID_NO_4878 | | Useful for making plants with |
| | | modulated nitrogen use efficiency |
| SEQ_ID_NO_4888 | | Useful for making plants with altered |
| | | cell wall content and/or composition |
| SEQ_ID_NO_4888 | | Useful for biocontainment applications |
| SEQ_ID_NO_4888 | The plant produces a reduced number | Useful for biocontainment applications |
| | of seeds. | |
| SEQ_ID_NO_4888 | Useful for making plants with | The sequence is a functional homolog |
| | modulated growth and phenotype | of SEQ ID NO 970 in the 11/435,388 |
| | characteristics | US patent application |
| SEQ_ID_NO_4888 | Useful for making plants with | The sequence is a functional homolog |
| | modulated growth and phenotype | of SEQ ID NO 72 in the 10/225,066 |
| | characteristics | US patent application |
| SEQ_ID_NO_4888 | Useful for making plants with | The sequence is a functional homolog |
| | modulated growth and phenotype | of SEQ ID NO 2624 in the 10/374,780 |
| | characteristics | US patent application |
| SEQ_ID_NO_4888 | Useful for making plants with | The sequence is a functional homolog |
| | modulated growth and phenotype | of SEQ ID NO 1520 in the 10/666,642 |
| | characteristics | US patent application |
| SEQ_ID_NO_4930 | Useful for making plants with | The sequence is a functional homolog |
| | modulated growth and phenotype | of SEQ ID NO 614 in the 10/666,642 |
| | characteristics | US patent application |
| SEQ_ID_NO_4940 | The plant flowers significantly earllier | Useful for making plants with |
| | than wild-type. | modulated flowering time |
| SEQ_ID_NO_4940 | | Useful for making plants with |
| | | modulated flowering time |
| SEQ_ID_NO_4940 | The plant is abnormally small. | Useful for making plants with |
| | | modulated biomass |
| SEQ_ID_NO_4940 | | Useful for making plants with |
| | | modulated biomass |
| SEQ_ID_NO_4942 | The plant is abnormally large. | Useful for making plants with |
| | | modulated biomass |
| SEQ_ID_NO_4950 | | Useful for making plants with altered oil |
| | | content |
| SEQ_ID_NO_4978 | | Useful for making plants with |
| | | modulated nitrogen use efficiency |
| SEQ_ID_NO_4978 | The plant is abnormally small. | Useful for making plants with |
| | | modulated biomass |
| SEQ_ID_NO_4978 | | Useful for making plants with altered |
| | | Lignin content |
| SEQ_ID_NO_4978 | | Useful for making plants with altered |
| | | alkaloid content |
| SEQ_ID_NO_4978 | Useful for making plants with | The sequence is a functional homolog |
| | modulated growth and phenotype | of SEQ ID NO 1016 in the 11/435,388 |
| | characteristics | US patent application |
| SEQ_ID_NO_4982 | | Useful for making plants with altered |
| | | Lignin content |
| SEQ_ID_NO_5014 | The plant is abnormally small. | Useful for making plants with |
| | | modulated biomass |
| SEQ_ID_NO_5020 | | Useful for making plants with altered |
| | | Lignin content |
| SEQ_ID_NO_5024 | | Useful for making plants with altered |
| | | Lignin content |
| SEQ_ID_NO_5024 | | Useful for making plants with altered |
| | | alkaloid content |
| SEQ_ID_NO_5036 | | Useful for making plants with |
| | | modulated heat sensitivity |
| SEQ_ID_NO_5036 | | Useful for making plants with altered |
| | | Lignin content |
| SEQ_ID_NO_5038 | | Useful for making plants with altered |
| | | cell wall content and/or composition |
| SEQ_ID_NO_5056 | The plant produces a reduced number | Useful for biocontainment applications |
| | of seeds. | |
| SEQ_ID_NO_5068 | The plant flowers significantly late | Useful for making plants with |
| | compared to wild-type. | modulated flowering time |
| SEQ_ID_NO_5068 | No seed is being produced by the plant | Useful for biocontainment applications |
| SEQ_ID_NO_5100 | The plant flowers significantly late | Useful for making plants with |
| | compared to wild-type. | modulated flowering time |
| SEQ_ID_NO_5100 | | Useful for making plants with |
| | | modulated cold sensitivity |
| SEQ_ID_NO_5114 | The plant is abnormally large. | Useful for making plants with |
| | | modulated biomass |
| SEQ_ID_NO_5114 | The plant is abnormally small. | Useful for making plants with |
| | | modulated biomass |
| SEQ_ID_NO_5118 | The plant flowers significantly late | Useful for making plants with |
| | compared to wild-type. | modulated flowering time |
| SEQ_ID_NO_5118 | The plant is abnormally small. | Useful for making plants with |
| | | modulated biomass |
| SEQ_ID_NO_5136 | The plant is abnormally large. | Useful for making plants with |
| | | modulated biomass |
| SEQ_ID_NO_5136 | The plant is abnormally small. | Useful for making plants with |
| | | modulated biomass |
| SEQ_ID_NO_5184 | The plant flowers significantly late | Useful for making plants with |
| | compared to wild-type. | modulated flowering time |
| SEQ_ID_NO_5184 | | Useful for making plants with |
| | | modulated cold sensitivity |
| SEQ_ID_NO_5188 | | Useful for making plants with altered |
| | | cell wall content and/or composition |
| SEQ_ID_NO_5196 | The plant is abnormally small. | Useful for making plants with |
| | | modulated biomass |
| SEQ_ID_NO_5240 | | Useful for making plants with |
| | | modulated drought sensitivity |
| SEQ_ID_NO_5248 | | Useful for making plants with altered |
| | | Lignin content |
| SEQ_ID_NO_5248 | | Useful for making plants with altered |
| | | alkaloid content |
| SEQ_ID_NO_5262 | The plant flowers significantly late | Useful for making plants with |
| | compared to wild-type. | modulated flowering time |
| SEQ_ID_NO_5274 | | Useful for making plants with |
| | | modulated flowering time |
| SEQ_ID_NO_5304 | | Useful for making plants with |
| | | modulated phosphate use efficiency |
| SEQ_ID_NO_5304 | | Useful for making plants with |
| | | modulated drought sensitivity |
| SEQ_ID_NO_5304 | | Useful for making plants with |
| | | modulated biomass |
| SEQ_ID_NO_5316 | The plant flowers significantly late | Useful for making plants with |
| | compared to wild-type. | modulated flowering time |
| SEQ_ID_NO_5316 | No seed is being produced by the plant | Useful for biocontainment applications |
| SEQ_ID_NO_5366 | The plant is abnormally small. | Useful for making plants with |
| | | modulated biomass |
| SEQ_ID_NO_5370 | | Useful for making plants with altered |
| | | terpenoid content |
| SEQ_ID_NO_5370 | | Useful for making plants with altered |
| | | Lignin content |
| SEQ_ID_NO_5372 | The plant flowers significantly late | Useful for making plants with |
| | compared to wild-type. | modulated flowering time |
| SEQ_ID_NO_5372 | No seed is being produced by the plant | Useful for biocontainment applications |
| SEQ_ID_NO_5380 | The plant flowers significantly late | Useful for making plants with |
| | compared to wild-type. | modulated flowering time |
| SEQ_ID_NO_5380 | No seed is being produced by the plant | Useful for biocontainment applications |
| SEQ_ID_NO_5398 | The plant senesces significantly late | Useful for making plants with |
| | copared to wild-type. | modulated time to senescence |
| SEQ_ID_NO_5422 | | Useful for making plants with |
| | | modulated nitrogen use efficiency |
| SEQ_ID_NO_5432 | The plant is abnormally small. | Useful for making plants with |
| | | modulated biomass |
| SEQ_ID_NO_5460 | | Useful for making plants with |
| | | modulated drought sensitivity |
| SEQ_ID_NO_5460 | Useful for making plants with | The sequence is a functional homolog |
| | modulated growth and phenotype | of SEQ ID NO 805 in the 11/479,226 |
| | characteristics | US patent application |
| SEQ_ID_NO_5460 | Useful for making plants with | The sequence is a functional homolog |
| | modulated growth and phenotype | of SEQ ID NO 801 in the 11/479,226 |
| | characteristics | US patent application |
| SEQ_ID_NO_5460 | Useful for making plants with | The sequence is a functional homolog |
| | modulated growth and phenotype | of SEQ ID NO 797 in the 11/479,226 |
| | characteristics | US patent application |
| SEQ_ID_NO_5460 | Useful for making plants with | The sequence is a functional homolog |
| | modulated growth and phenotype | of SEQ ID NO 790 in the 10/225,066 |
| | characteristics | US patent application |
| SEQ_ID_NO_5460 | Useful for making plants with | The sequence is a functional homolog |
| | modulated growth and phenotype | of SEQ ID NO 789 in the 11/479,226 |
| | characteristics | US patent application |
| SEQ_ID_NO_5460 | Useful for making plants with | The sequence is a functional homolog |
| | modulated growth and phenotype | of SEQ ID NO 786 in the 11/479,226 |
| | characteristics | US patent application |
| SEQ_ID_NO_5460 | Useful for making plants with | The sequence is a functional homolog |
| | modulated growth and phenotype | of SEQ ID NO 782 in the 11/479,226 |
| | characteristics | US patent application |
| SEQ_ID_NO_5460 | Useful for making plants with | The sequence is a functional homolog |
| | modulated growth and phenotype | of SEQ ID NO 344 in the 10/412,699 |
| | characteristics | US patent application |
| SEQ_ID_NO_5460 | Useful for making plants with | The sequence is a functional homolog |
| | modulated growth and phenotype | of SEQ ID NO 342 in the 10/412,699 |
| | characteristics | US patent application |
| SEQ_ID_NO_5460 | Useful for making plants with | The sequence is a functional homolog |
| | modulated growth and phenotype | of SEQ ID NO 2350 in the 10/374,780 |
| | characteristics | US patent application |
| SEQ_ID_NO_5462 | | Useful for making plants with altered |
| | | terpenoid content |
| SEQ_ID_NO_5588 | The plant produces a reduced number | Useful for biocontainment applications |
| | of seeds. | |
| SEQ_ID_NO_5600 | The plant is abnormally small. | Useful for making plants with |
| | | modulated biomass |
| SEQ_ID_NO_5600 | | Useful for making plants with altered |
| | | cell wall content and/or composition |
| SEQ_ID_NO_5664 | The plant is abnormally small. | Useful for making plants with |
| | | modulated biomass |
| SEQ_ID_NO_5708 | | Useful for making plants with |
| | | modulated phosphate use efficiency |
| SEQ_ID_NO_5708 | The plant flowers significantly late | Useful for making plants with |
| | compared to wild-type. | modulated flowering time |
| SEQ_ID_NO_5708 | The plant is abnormally large. | Useful for making plants with |
| | | modulated biomass |
| SEQ_ID_NO_5710 | The plant is abnormally small. | Useful for making plants with |
| | | modulated biomass |
| SEQ_ID_NO_5710 | The leaves start out wild-type green | Useful for biocontainment applications |
| | and gradually turn yellow-green in | |
| | color, while the cotyledons stay wild- | |
| | type green. | |
| SEQ_ID_NO_5746 | | Useful for making plants with altered |
| | | terpenoid content |
| SEQ_ID_NO_5748 | | Useful for making plants with |
| | | modulated phosphate use efficiency |
| SEQ_ID_NO_5748 | The plant flowers significantly late | Useful for making plants with |
| | compared to wild-type. | modulated flowering time |
| SEQ_ID_NO_5748 | The plant is abnormally large. | Useful for making plants with |
| | | modulated biomass |
| SEQ_ID_NO_5808 | There is no branching. | Useful for making plants with |
| | | modulated biomass |
| SEQ_ID_NO_5840 | Useful for making plants with | The sequence is a functional homolog |
| | modulated growth and phenotype | of SEQ ID NO 92 in the 10/374,780 |
| | characteristics | US patent application |
| SEQ_ID_NO_5840 | Useful for making plants with | The sequence is a functional homolog |
| | modulated growth and phenotype | of SEQ ID NO 824 in the 10/374,780 |
| | characteristics | US patent application |
| SEQ_ID_NO_5840 | Useful for making plants with | The sequence is a functional homolog |
| | modulated growth and phenotype | of SEQ ID NO 715 in the 11/479,226 |
| | characteristics | US patent application |
| SEQ_ID_NO_5840 | Useful for making plants with | The sequence is a functional homolog |
| | modulated growth and phenotype | of SEQ ID NO 24 in the 11/375,241 |
| | characteristics | US patent application |
| SEQ_ID_NO_5840 | Useful for making plants with | The sequence is a functional homolog |
| | modulated growth and phenotype | of SEQ ID NO 2190 in the 10/374,780 |
| | characteristics | US patent application |
| SEQ_ID_NO_5850 | The plant flowers significantly earllier | Useful for making plants with |
| | than wild-type. | modulated flowering time |
| SEQ_ID_NO_5850 | | Useful for making plants with |
| | | modulated flowering time |
| SEQ_ID_NO_5850 | The plant is abnormally small. | Useful for making plants with |
| | | modulated biomass |
| SEQ_ID_NO_5850 | | Useful for making plants with |
| | | modulated biomass |
| SEQ_ID_NO_5872 | | Useful for making plants with |
| | | modulated drought sensitivity |
| SEQ_ID_NO_5872 | Useful for making plants with | The sequence is a functional homolog |
| | modulated growth and phenotype | of SEQ ID NO 805 in the 11/479,226 |
| | characteristics | US patent application |
| SEQ_ID_NO_5872 | Useful for making plants with | The sequence is a functional homolog |
| | modulated growth and phenotype | of SEQ ID NO 801 in the 11/479,226 |
| | characteristics | US patent application |
| SEQ_ID_NO_5872 | Useful for making plants with | The sequence is a functional homolog |
| | modulated growth and phenotype | of SEQ ID NO 797 in the 11/479,226 |
| | characteristics | US patent application |
| SEQ_ID_NO_5872 | Useful for making plants with | The sequence is a functional homolog |
| | modulated growth and phenotype | of SEQ ID NO 790 in the 10/225,066 |
| | characteristics | US patent application |
| SEQ_ID_NO_5872 | Useful for making plants with | The sequence is a functional homolog |
| | modulated growth and phenotype | of SEQ ID NO 789 in the 11/479,226 |
| | characteristics | US patent application |
| SEQ_ID_NO_5872 | Useful for making plants with | The sequence is a functional homolog |
| | modulated growth and phenotype | of SEQ ID NO 786 in the 11/479,226 |
| | characteristics | US patent application |
| SEQ_ID_NO_5872 | Useful for making plants with | The sequence is a functional homolog |
| | modulated growth and phenotype | of SEQ ID NO 782 in the 11/479,226 |
| | characteristics | US patent application |
| SEQ_ID_NO_5872 | Useful for making plants with | The sequence is a functional homolog |
| | modulated growth and phenotype | of SEQ ID NO 344 in the 10/412,699 |
| | characteristics | US patent application |
| SEQ_ID_NO_5872 | Useful for making plants with | The sequence is a functional homolog |
| | modulated growth and phenotype | of SEQ ID NO 342 in the 10/412,699 |
| | characteristics | US patent application |
| SEQ_ID_NO_5872 | Useful for making plants with | The sequence is a functional homolog |
| | modulated growth and phenotype | of SEQ ID NO 2350 in the 10/374,780 |
| | characteristics | US patent application |
| SEQ_ID_NO_5872 | Useful for making plants with | The sequence is a functional homolog |
| | modulated growth and phenotype | of SEQ ID NO 1777 in the 11/479,226 |
| | characteristics | US patent application |
| SEQ_ID_NO_5876 | The plant flowers significantly earllier | Useful for making plants with |
| | than wild-type. | modulated flowering time |
| SEQ_ID_NO_5876 | | Useful for making plants with |
| | | modulated flowering time |
| SEQ_ID_NO_5876 | The plant is abnormally small. | Useful for making plants with |
| | | modulated biomass |
| SEQ_ID_NO_5876 | | Useful for making plants with |
| | | modulated biomass |
| SEQ_ID_NO_5882 | The plant is abnormally large. | Useful for making plants with |
| | | modulated biomass |
| SEQ_ID_NO_5882 | The plant is abnormally small. | Useful for making plants with |
| | | modulated biomass |
| SEQ_ID_NO_5886 | The plant is abnormally large. | Useful for making plants with |
| | | modulated biomass |
| SEQ_ID_NO_5886 | Useful for making plants with | The sequence is a functional homolog |
| | modulated growth and phenotype | of SEQ ID NO 786 in the 10/412,699 |
| | characteristics | US patent application |
| SEQ_ID_NO_5886 | Useful for making plants with | The sequence is a functional homolog |
| | modulated growth and phenotype | of SEQ ID NO 784 in the 10/412,699 |
| | characteristics | US patent application |
| SEQ_ID_NO_5886 | Useful for making plants with | The sequence is a functional homolog |
| | modulated growth and phenotype | of SEQ ID NO 1617 in the 11/479,226 |
| | characteristics | US patent application |
| SEQ_ID_NO_5886 | Useful for making plants with | The sequence is a functional homolog |
| | modulated growth and phenotype | of SEQ ID NO 1614 in the 11/479,226 |
| | characteristics | US patent application |
| SEQ_ID_NO_5894 | The plant flowers significantly late | Useful for making plants with |
| | compared to wild-type. | modulated flowering time |
| SEQ_ID_NO_5900 | | Useful for making plants with |
| | | modulated flowering time |
| SEQ_ID_NO_5910 | The plant flowers significantly late | Useful for making plants with |
| | compared to wild-type. | modulated flowering time |
| SEQ_ID_NO_5910 | No seed is being produced by the plant | Useful for biocontainment applications |
| SEQ_ID_NO_5926 | | Useful for making plants with |
| | | modulated heat sensitivity |
| SEQ_ID_NO_5926 | | Useful for making plants with altered |
| | | Lignin content |
| SEQ_ID_NO_5932 | The plant produces a reduced number | Useful for biocontainment applications |
| | of seeds. | |
| SEQ_ID_NO_5970 | There is no branching. | Useful for making plants with |
| | | modulated biomass |
| SEQ_ID_NO_5972 | | Useful for making plants with altered |
| | | Lignin content |
| SEQ_ID_NO_5978 | The plant senesces significantly late | Useful for making plants with |
| | copared to wild-type. | modulated time to senescence |
| SEQ_ID_NO_5978 | The plant flowers significantly late | Useful for making plants with |
| | compared to wild-type. | modulated flowering time |
| SEQ_ID_NO_5990 | The plant is abnormally large. | Useful for making plants with |
| | | modulated biomass |
| SEQ_ID_NO_6002 | The plant is abnormally small. | Useful for making plants with |
| | | modulated biomass |
| SEQ_ID_NO_6002 | The plant produces a reduced number | Useful for biocontainment applications |
| | of seeds. | |
| SEQ_ID_NO_6008 | | Useful for making plants with |
| | | modulated drought sensitivity |
| SEQ_ID_NO_6008 | Useful for making plants with | The sequence is a functional homolog |
| | modulated growth and phenotype | of SEQ ID NO 805 in the 11/479,226 |
| | characteristics | US patent application |
| SEQ_ID_NO_6008 | Useful for making plants with | The sequence is a functional homolog |
| | modulated growth and phenotype | of SEQ ID NO 801 in the 11/479,226 |
| | characteristics | US patent application |
| SEQ_ID_NO_6008 | Useful for making plants with | The sequence is a functional homolog |
| | modulated growth and phenotype | of SEQ ID NO 797 in the 11/479,226 |
| | characteristics | US patent application |
| SEQ_ID_NO_6008 | Useful for making plants with | The sequence is a functional homolog |
| | modulated growth and phenotype | of SEQ ID NO 790 in the 10/225,066 |
| | characteristics | US patent application |
| SEQ_ID_NO_6008 | Useful for making plants with | The sequence is a functional homolog |
| | modulated growth and phenotype | of SEQ ID NO 789 in the 11/479,226 |
| | characteristics | US patent application |
| SEQ_ID_NO_6008 | Useful for making plants with | The sequence is a functional homolog |
| | modulated growth and phenotype | of SEQ ID NO 786 in the 11/479,226 |
| | characteristics | US patent application |
| SEQ_ID_NO_6008 | Useful for making plants with | The sequence is a functional homolog |
| | modulated growth and phenotype | of SEQ ID NO 782 in the 11/479,226 |
| | characteristics | US patent application |
| SEQ_ID_NO_6008 | Useful for making plants with | The sequence is a functional homolog |
| | modulated growth and phenotype | of SEQ ID NO 344 in the 10/412,699 |
| | characteristics | US patent application |
| SEQ_ID_NO_6008 | Useful for making plants with | The sequence is a functional homolog |
| | modulated growth and phenotype | of SEQ ID NO 342 in the 10/412,699 |
| | characteristics | US patent application |
| SEQ_ID_NO_6008 | Useful for making plants with | The sequence is a functional homolog |
| | modulated growth and phenotype | of SEQ ID NO 2350 in the 10/374,780 |
| | characteristics | US patent application |
| SEQ_ID_NO_602 | The plant flowers significantly late | Useful for making plants with |
| | compared to wild-type. | modulated flowering time |
| SEQ_ID_NO_602 | No seed is being produced by the plant | Useful for biocontainment applications |
| SEQ_ID_NO_6032 | | Useful for making plants with altered |
| | | terpenoid content |
| SEQ_ID_NO_6032 | | Useful for making plants with altered |
| | | Lignin content |
| SEQ_ID_NO_6044 | | Useful for making plants with altered |
| | | Lignin content |
| SEQ_ID_NO_6058 | | Useful for making plants with |
| | | modulated flowering time |
| SEQ_ID_NO_6132 | There is no branching. | Useful for making plants with |
| | | modulated biomass |
| SEQ_ID_NO_6134 | The plant is abnormally large. | Useful for making plants with |
| | | modulated biomass |
| SEQ_ID_NO_6134 | The plant is abnormally small. | Useful for making plants with |
| | | modulated biomass |
| SEQ_ID_NO_6156 | | Useful for making plants with |
| | | modulated cold sensitivity |
| SEQ_ID_NO_6166 | | Useful for making plants with altered |
| | | Lignin content |
| SEQ_ID_NO_6194 | | Useful for making plants with |
| | | modulated drought sensitivity |
| SEQ_ID_NO_6194 | Useful for making plants with | The sequence is a functional homolog |
| | modulated growth and phenotype | of SEQ ID NO 805 in the 11/479,226 |
| | characteristics | US patent application |
| SEQ_ID_NO_6194 | Useful for making plants with | The sequence is a functional homolog |
| | modulated growth and phenotype | of SEQ ID NO 801 in the 11/479,226 |
| | characteristics | US patent application |
| SEQ_ID_NO_6194 | Useful for making plants with | The sequence is a functional homolog |
| | modulated growth and phenotype | of SEQ ID NO 797 in the 11/479,226 |
| | characteristics | US patent application |
| SEQ_ID_NO_6194 | Useful for making plants with | The sequence is a functional homolog |
| | modulated growth and phenotype | of SEQ ID NO 790 in the 10/225,066 |
| | characteristics | US patent application |
| SEQ_ID_NO_6194 | Useful for making plants with | The sequence is a functional homolog |
| | modulated growth and phenotype | of SEQ ID NO 789 in the 11/479,226 |
| | characteristics | US patent application |
| SEQ_ID_NO_6194 | Useful for making plants with | The sequence is a functional homolog |
| | modulated growth and phenotype | of SEQ ID NO 786 in the 11/479,226 |
| | characteristics | US patent application |
| SEQ_ID_NO_6194 | Useful for making plants with | The sequence is a functional homolog |
| | modulated growth and phenotype | of SEQ ID NO 782 in the 11/479,226 |
| | characteristics | US patent application |
| SEQ_ID_NO_6194 | Useful for making plants with | The sequence is a functional homolog |
| | modulated growth and phenotype | of SEQ ID NO 344 in the 10/412,699 |
| | characteristics | US patent application |
| SEQ_ID_NO_6194 | Useful for making plants with | The sequence is a functional homolog |
| | modulated growth and phenotype | of SEQ ID NO 342 in the 10/412,699 |
| | characteristics | US patent application |
| SEQ_ID_NO_6194 | Useful for making plants with | The sequence is a functional homolog |
| | modulated growth and phenotype | of SEQ ID NO 2350 in the 10/374,780 |
| | characteristics | US patent application |
| SEQ_ID_NO_6194 | Useful for making plants with | The sequence is a functional homolog |
| | modulated growth and phenotype | of SEQ ID NO 1777 in the 11/479,226 |
| | characteristics | US patent application |
| SEQ_ID_NO_6200 | | Useful for making plants with |
| | | modulated phosphate use efficiency |
| SEQ_ID_NO_6200 | | Useful for making plants with |
| | | modulated nitrogen use efficiency |
| SEQ_ID_NO_6220 | The plant is abnormally large. | Useful for making plants with |
| | | modulated biomass |
| SEQ_ID_NO_6236 | The plant senesces significantly late | Useful for making plants with |
| | copared to wild-type. | modulated time to senescence |
| SEQ_ID_NO_6256 | | Useful for making plants with |
| | | modulated drought sensitivity |
| SEQ_ID_NO_6256 | Useful for making plants with | The sequence is a functional homolog |
| | modulated growth and phenotype | of SEQ ID NO 805 in the 11/479,226 |
| | characteristics | US patent application |
| SEQ_ID_NO_6256 | Useful for making plants with | The sequence is a functional homolog |
| | modulated growth and phenotype | of SEQ ID NO 801 in the 11/479,226 |
| | characteristics | US patent application |
| SEQ_ID_NO_6256 | Useful for making plants with | The sequence is a functional homolog |
| | modulated growth and phenotype | of SEQ ID NO 797 in the 11/479,226 |
| | characteristics | US patent application |
| SEQ_ID_NO_6256 | Useful for making plants with | The sequence is a functional homolog |
| | modulated growth and phenotype | of SEQ ID NO 790 in the 10/225,066 |
| | characteristics | US patent application |
| SEQ_ID_NO_6256 | Useful for making plants with | The sequence is a functional homolog |
| | modulated growth and phenotype | of SEQ ID NO 789 in the 11/479,226 |
| | characteristics | US patent application |
| SEQ_ID_NO_6256 | Useful for making plants with | The sequence is a functional homolog |
| | modulated growth and phenotype | of SEQ ID NO 786 in the 11/479,226 |
| | characteristics | US patent application |
| SEQ_ID_NO_6256 | Useful for making plants with | The sequence is a functional homolog |
| | modulated growth and phenotype | of SEQ ID NO 782 in the 11/479,226 |
| | characteristics | US patent application |
| SEQ_ID_NO_6256 | Useful for making plants with | The sequence is a functional homolog |
| | modulated growth and phenotype | of SEQ ID NO 344 in the 10/412,699 |
| | characteristics | US patent application |
| SEQ_ID_NO_6256 | Useful for making plants with | The sequence is a functional homolog |
| | modulated growth and phenotype | of SEQ ID NO 342 in the 10/412,699 |
| | characteristics | US patent application |
| SEQ_ID_NO_6256 | Useful for making plants with | The sequence is a functional homolog |
| | modulated growth and phenotype | of SEQ ID NO 2350 in the 10/374,780 |
| | characteristics | US patent application |
| SEQ_ID_NO_6304 | | Useful for making plants with altered |
| | | terpenoid content |
| SEQ_ID_NO_632 | | Useful for making plants with |
| | | modulated nitrogen use efficiency |
| SEQ_ID_NO_632 | | Useful for making plants with altered |
| | | Lignin content |
| SEQ_ID_NO_6320 | | Useful for making plants with altered |
| | | cell wall content and/or composition |
| SEQ_ID_NO_6326 | The plant flowers significantly late | Useful for making plants with |
| | compared to wild-type. | modulated flowering time |
| SEQ_ID_NO_6326 | The plant produces a reduced number | Useful for biocontainment applications |
| | of seeds. | |
| SEQ_ID_NO_636 | Useful for making plants with | The sequence is a functional homolog |
| | modulated growth and phenotype | of SEQ ID NO 186 in the 11/435,388 |
| | characteristics | US patent application |
| SEQ_ID_NO_636 | Useful for making plants with | The sequence is a functional homolog |
| | modulated growth and phenotype | of SEQ ID NO 186 in the 10/714,887 |
| | characteristics | US patent application |
| SEQ_ID_NO_636 | Useful for making plants with | The sequence is a functional homolog |
| | modulated growth and phenotype | of SEQ ID NO 174 in the 11/435,388 |
| | characteristics | US patent application |
| SEQ_ID_NO_636 | Useful for making plants with | The sequence is a functional homolog |
| | modulated growth and phenotype | of SEQ ID NO 174 in the 10/714,887 |
| | characteristics | US patent application |
| SEQ_ID_NO_636 | Useful for making plants with | The sequence is a functional homolog |
| | modulated growth and phenotype | of SEQ ID NO 172 in the 11/435,388 |
| | characteristics | US patent application |
| SEQ_ID_NO_636 | Useful for making plants with | The sequence is a functional homolog |
| | modulated growth and phenotype | of SEQ ID NO 172 in the 10/714,887 |
| | characteristics | US patent application |
| SEQ_ID_NO_6372 | | Useful for making plants with |
| | | modulated flowering time |
| SEQ_ID_NO_6372 | | Useful for making plants with altered |
| | | Lignin content |
| SEQ_ID_NO_6372 | Useful for making plants with | The sequence is a functional homolog |
| | modulated growth and phenotype | of SEQ ID NO 780 in the 10/412,699 |
| | characteristics | US patent application |
| SEQ_ID_NO_6372 | Useful for making plants with | The sequence is a functional homolog |
| | modulated growth and phenotype | of SEQ ID NO 1611 in the 11/479,226 |
| | characteristics | US patent application |
| SEQ_ID_NO_638 | Useful for making plants with | The sequence is a functional homolog |
| | modulated growth and phenotype | of SEQ ID NO 92 in the 10/669,824 |
| | characteristics | US patent application |
| SEQ_ID_NO_638 | Useful for making plants with | The sequence is a functional homolog |
| | modulated growth and phenotype | of SEQ ID NO 90 in the 10/669,824 |
| | characteristics | US patent application |
| SEQ_ID_NO_638 | Useful for making plants with | The sequence is a functional homolog |
| | modulated growth and phenotype | of SEQ ID NO 88 in the 10/669,824 |
| | characteristics | US patent application |
| SEQ_ID_NO_638 | Useful for making plants with | The sequence is a functional homolog |
| | modulated growth and phenotype | of SEQ ID NO 66 in the 10/870,198 |
| | characteristics | US patent application |
| SEQ_ID_NO_638 | Useful for making plants with | The sequence is a functional homolog |
| | modulated growth and phenotype | of SEQ ID NO 66 in the 10/669,824 |
| | characteristics | US patent application |
| SEQ_ID_NO_638 | Useful for making plants with | The sequence is a functional homolog |
| | modulated growth and phenotype | of SEQ ID NO 38 in the 10/870,198 |
| | characteristics | US patent application |
| SEQ_ID_NO_638 | Useful for making plants with | The sequence is a functional homolog |
| | modulated growth and phenotype | of SEQ ID NO 38 in the 10/669,824 |
| | characteristics | US patent application |
| SEQ_ID_NO_638 | Useful for making plants with | The sequence is a functional homolog |
| | modulated growth and phenotype | of SEQ ID NO 348 in the 10/714,887 |
| | characteristics | US patent application |
| SEQ_ID_NO_638 | Useful for making plants with | The sequence is a functional homolog |
| | modulated growth and phenotype | of SEQ ID NO 346 in the 11/435,388 |
| | characteristics | US patent application |
| SEQ_ID_NO_638 | Useful for making plants with | The sequence is a functional homolog |
| | modulated growth and phenotype | of SEQ ID NO 346 in the 10/714,887 |
| | characteristics | US patent application |
| SEQ_ID_NO_638 | Useful for making plants with | The sequence is a functional homolog |
| | modulated growth and phenotype | of SEQ ID NO 344 in the 11/435,388 |
| | characteristics | US patent application |
| SEQ_ID_NO_638 | Useful for making plants with | The sequence is a functional homolog |
| | modulated growth and phenotype | of SEQ ID NO 22 in the 10/870,198 |
| | characteristics | US patent application |
| SEQ_ID_NO_638 | Useful for making plants with | The sequence is a functional homolog |
| | modulated growth and phenotype | of SEQ ID NO 22 in the 10/669,824 |
| | characteristics | US patent application |
| SEQ_ID_NO_638 | Useful for making plants with | The sequence is a functional homolog |
| | modulated growth and phenotype | of SEQ ID NO 1507 in the 10/412,699 |
| | characteristics | US patent application |
| SEQ_ID_NO_638 | Useful for making plants with | The sequence is a functional homolog |
| | modulated growth and phenotype | of SEQ ID NO 1413 in the 10/374,780 |
| | characteristics | US patent application |
| SEQ_ID_NO_638 | Useful for making plants with | The sequence is a functional homolog |
| | modulated growth and phenotype | of SEQ ID NO 1410 in the 10/374,780 |
| | characteristics | US patent application |
| SEQ_ID_NO_638 | Useful for making plants with | The sequence is a functional homolog |
| | modulated growth and phenotype | of SEQ ID NO 1402 in the 10/374,780 |
| | characteristics | US patent application |
| SEQ_ID_NO_638 | Useful for making plants with | The sequence is a functional homolog |
| | modulated growth and phenotype | of SEQ ID NO 1395 in the 10/374,780 |
| | characteristics | US patent application |
| SEQ_ID_NO_6412 | The plant is abnormally small. | Useful for making plants with |
| | | modulated biomass |
| SEQ_ID_NO_6412 | | Useful for making plants with altered |
| | | alkaloid content |
| SEQ_ID_NO_6414 | | Useful for making plants with |
| | | modulated drought sensitivity |
| SEQ_ID_NO_6414 | Useful for making plants with | The sequence is a functional homolog |
| | modulated growth and phenotype | of SEQ ID NO 805 in the 11/479,226 |
| | characteristics | US patent application |
| SEQ_ID_NO_6414 | Useful for making plants with | The sequence is a functional homolog |
| | modulated growth and phenotype | of SEQ ID NO 801 in the 11/479,226 |
| | characteristics | US patent application |
| SEQ_ID_NO_6414 | Useful for making plants with | The sequence is a functional homolog |
| | modulated growth and phenotype | of SEQ ID NO 797 in the 11/479,226 |
| | characteristics | US patent application |
| SEQ_ID_NO_6414 | Useful for making plants with | The sequence is a functional homolog |
| | modulated growth and phenotype | of SEQ ID NO 790 in the 10/225,066 |
| | characteristics | US patent application |
| SEQ_ID_NO_6414 | Useful for making plants with | The sequence is a functional homolog |
| | modulated growth and phenotype | of SEQ ID NO 789 in the 11/479,226 |
| | characteristics | US patent application |
| SEQ_ID_NO_6414 | Useful for making plants with | The sequence is a functional homolog |
| | modulated growth and phenotype | of SEQ ID NO 786 in the 11/479,226 |
| | characteristics | US patent application |
| SEQ_ID_NO_6414 | Useful for making plants with | The sequence is a functional homolog |
| | modulated growth and phenotype | of SEQ ID NO 782 in the 11/479,226 |
| | characteristics | US patent application |
| SEQ_ID_NO_6414 | Useful for making plants with | The sequence is a functional homolog |
| | modulated growth and phenotype | of SEQ ID NO 344 in the 10/412,699 |
| | characteristics | US patent application |
| SEQ_ID_NO_6414 | Useful for making plants with | The sequence is a functional homolog |
| | modulated growth and phenotype | of SEQ ID NO 342 in the 10/412,699 |
| | characteristics | US patent application |
| SEQ_ID_NO_6414 | Useful for making plants with | The sequence is a functional homolog |
| | modulated growth and phenotype | of SEQ ID NO 2350 in the 10/374,780 |
| | characteristics | US patent application |
| SEQ_ID_NO_6424 | | Useful for making plants with |
| | | modulated drought sensitivity |
| SEQ_ID_NO_6424 | Useful for making plants with | The sequence is a functional homolog |
| | modulated growth and phenotype | of SEQ ID NO 805 in the 11/479,226 |
| | characteristics | US patent application |
| SEQ_ID_NO_6424 | Useful for making plants with | The sequence is a functional homolog |
| | modulated growth and phenotype | of SEQ ID NO 801 in the 11/479,226 |
| | characteristics | US patent application |
| SEQ_ID_NO_6424 | Useful for making plants with | The sequence is a functional homolog |
| | modulated growth and phenotype | of SEQ ID NO 797 in the 11/479,226 |
| | characteristics | US patent application |
| SEQ_ID_NO_6424 | Useful for making plants with | The sequence is a functional homolog |
| | modulated growth and phenotype | of SEQ ID NO 790 in the 10/225,066 |
| | characteristics | US patent application |
| SEQ_ID_NO_6424 | Useful for making plants with | The sequence is a functional homolog |
| | modulated growth and phenotype | of SEQ ID NO 789 in the 11/479,226 |
| | characteristics | US patent application |
| SEQ_ID_NO_6424 | Useful for making plants with | The sequence is a functional homolog |
| | modulated growth and phenotype | of SEQ ID NO 786 in the 11/479,226 |
| | characteristics | US patent application |
| SEQ_ID_NO_6424 | Useful for making plants with | The sequence is a functional homolog |
| | modulated growth and phenotype | of SEQ ID NO 782 in the 11/479,226 |
| | characteristics | US patent application |
| SEQ_ID_NO_6424 | Useful for making plants with | The sequence is a functional homolog |
| | modulated growth and phenotype | of SEQ ID NO 344 in the 10/412,699 |
| | characteristics | US patent application |
| SEQ_ID_NO_6424 | Useful for making plants with | The sequence is a functional homolog |
| | modulated growth and phenotype | of SEQ ID NO 342 in the 10/412,699 |
| | characteristics | US patent application |
| SEQ_ID_NO_6424 | Useful for making plants with | The sequence is a functional homolog |
| | modulated growth and phenotype | of SEQ ID NO 2350 in the 10/374,780 |
| | characteristics | US patent application |
| SEQ_ID_NO_6430 | | Useful for making plants with |
| | | modulated oxidative stress sensitivity |
| SEQ_ID_NO_646 | The plant flowers significantly earllier | Useful for making plants with |
| | than wild-type. | modulated flowering time |
| SEQ_ID_NO_646 | The plant is abnormally small. | Useful for making plants with |
| | | modulated biomass |
| SEQ_ID_NO_646 | The plant produces a reduced number | Useful for biocontainment applications |
| | of seeds. | |
| SEQ_ID_NO_6496 | Useful for making plants with | The sequence is a functional homolog |
| | modulated growth and phenotype | of SEQ ID NO 32 in the 10/412,699 |
| | characteristics | US patent application |
| SEQ_ID_NO_6496 | Useful for making plants with | The sequence is a functional homolog |
| | modulated growth and phenotype | of SEQ ID NO 151 in the 11/479,226 |
| | characteristics | US patent application |
| SEQ_ID_NO_6518 | | Useful for making plants with |
| | | modulated oxidative stress sensitivity |
| SEQ_ID_NO_6524 | | Useful for making plants with altered |
| | | sterol content |
| SEQ_ID_NO_6538 | Useful for making plants with | The sequence is a functional homolog |
| | modulated growth and phenotype | of SEQ ID NO 542 in the 11/479,226 |
| | characteristics | US patent application |
| SEQ_ID_NO_6540 | | Useful for making plants with |
| | | modulated phosphate use efficiency |
| SEQ_ID_NO_6596 | The plant is abnormally small. | Useful for making plants with |
| | | modulated biomass |
| SEQ_ID_NO_6598 | | Useful for making plants with altered |
| | | Lignin content |
| SEQ_ID_NO_6598 | | Useful for making plants with altered |
| | | alkaloid content |
| SEQ_ID_NO_6604 | The plant flowers significantly late | Useful for making plants with |
| | compared to wild-type. | modulated flowering time |
| SEQ_ID_NO_6620 | The plant is abnormally large. | Useful for making plants with |
| | | modulated biomass |
| SEQ_ID_NO_6620 | The plant is abnormally small. | Useful for making plants with |
| | | modulated biomass |
| SEQ_ID_NO_6638 | The plant is abnormally small. | Useful for making plants with |
| | | modulated biomass |
| SEQ_ID_NO_6638 | The plant produces a reduced number | Useful for biocontainment applications |
| | of seeds. | |
| SEQ_ID_NO_6640 | The plant flowers significantly late | Useful for making plants with |
| | compared to wild-type. | modulated flowering time |
| SEQ_ID_NO_6640 | No seed is being produced by the plant | Useful for biocontainment applications |
| SEQ_ID_NO_6656 | The plant flowers significantly late | Useful for making plants with |
| | compared to wild-type. | modulated flowering time |
| SEQ_ID_NO_6656 | No seed is being produced by the plant | Useful for biocontainment applications |
| SEQ_ID_NO_6668 | | Useful for making plants with |
| | | modulated nitrogen use efficiency |
| SEQ_ID_NO_6696 | The plant flowers significantly late | Useful for making plants with |
| | compared to wild-type. | modulated flowering time |
| SEQ_ID_NO_6696 | The plant is abnormally small. | Useful for making plants with |
| | | modulated biomass |
| SEQ_ID_NO_6704 | The plant flowers significantly late | Useful for making plants with |
| | compared to wild-type. | modulated flowering time |
| SEQ_ID_NO_6704 | The plant is abnormally small. | Useful for making plants with |
| | | modulated biomass |
| SEQ_ID_NO_6712 | | Useful for making plants with |
| | | modulated nitrogen use efficiency |
| SEQ_ID_NO_6756 | | Useful for making plants with altered |
| | | Lignin content |
| SEQ_ID_NO_6758 | | Useful for making plants with |
| | | modulated nitrogen use efficiency |
| SEQ_ID_NO_6780 | The plant is abnormally small. | Useful for making plants with |
| | | modulated biomass |
| SEQ_ID_NO_6802 | | Useful for making plants with altered |
| | | terpenoid content |
| SEQ_ID_NO_6804 | | Useful for making plants with altered |
| | | Lignin content |
| SEQ_ID_NO_6820 | The plant flowers significantly late | Useful for making plants with |
| | compared to wild-type. | modulated flowering time |
| SEQ_ID_NO_6820 | No seed is being produced by the plant | Useful for biocontainment applications |
| SEQ_ID_NO_6836 | Useful for making plants with | The sequence is a functional homolog |
| | modulated growth and phenotype | of SEQ ID NO 1109 in the 10/374,780 |
| | characteristics | US patent application |
| SEQ_ID_NO_6836 | Useful for making plants with | The sequence is a functional homolog |
| | modulated growth and phenotype | of SEQ ID NO 1108 in the 10/374,780 |
| | characteristics | US patent application |
| SEQ_ID_NO_6894 | | Useful for biocontainment applications |
| SEQ_ID_NO_690 | | Useful for making plants with altered |
| | | terpenoid content |
| SEQ_ID_NO_690 | | Useful for making plants with altered |
| | | Lignin content |
| SEQ_ID_NO_6908 | | Useful for making plants with altered |
| | | terpenoid content |
| SEQ_ID_NO_6910 | The plant produces a reduced number | Useful for biocontainment applications |
| | of seeds. | |
| SEQ_ID_NO_6940 | The plant flowers significantly earllier | Useful for making plants with |
| | than wild-type. | modulated flowering time |
| SEQ_ID_NO_6940 | | Useful for making plants with |
| | | modulated flowering time |
| SEQ_ID_NO_6940 | The plant is abnormally small. | Useful for making plants with |
| | | modulated biomass |
| SEQ_ID_NO_6940 | | Useful for making plants with |
| | | modulated biomass |
| SEQ_ID_NO_6946 | The plant flowers significantly late | Useful for making plants with |
| | compared to wild-type. | modulated flowering time |
| SEQ_ID_NO_6946 | No seed is being produced by the plant | Useful for biocontainment applications |
| SEQ_ID_NO_6978 | | Useful for making plants with |
| | | modulated nitrogen use efficiency |
| SEQ_ID_NO_6986 | | Useful for making plants with |
| | | modulated nitrogen use efficiency |
| SEQ_ID_NO_7004 | | Useful for making plants with altered |
| | | terpenoid content |
| SEQ_ID_NO_7020 | | Useful for making plants with altered |
| | | terpenoid content |
| SEQ_ID_NO_7020 | | Useful for making plants with altered |
| | | alkaloid content |
| SEQ_ID_NO_7020 | Useful for making plants with | The sequence is a functional homolog |
| | modulated growth and phenotype | of SEQ ID NO 768 in the 10/412,699 |
| | characteristics | US patent application |
| SEQ_ID_NO_7032 | | Useful for making plants with altered |
| | | Lignin content |
| SEQ_ID_NO_7088 | The plant flowers significantly late | Useful for making plants with |
| | compared to wild-type. | modulated flowering time |
| SEQ_ID_NO_7088 | No seed is being produced by the plant | Useful for biocontainment applications |
| SEQ_ID_NO_7100 | | Useful for making plants with altered |
| | | Lignin content |
| SEQ_ID_NO_712 | | Useful for making plants with altered oil |
| | | content |
| SEQ_ID_NO_7126 | The plant flowers significantly late | Useful for making plants with |
| | compared to wild-type. | modulated flowering time |
| SEQ_ID_NO_7126 | No seed is being produced by the plant | Useful for biocontainment applications |
| SEQ_ID_NO_7142 | There is no branching. | Useful for making plants with |
| | | modulated biomass |
| SEQ_ID_NO_7156 | | Useful for making plants with |
| | | modulated nitrogen use efficiency |
| SEQ_ID_NO_7162 | The plant flowers significantly late | Useful for making plants with |
| | compared to wild-type. | modulated flowering time |
| SEQ_ID_NO_7162 | No seed is being produced by the plant | Useful for biocontainment applications |
| SEQ_ID_NO_7166 | The plant flowers significantly late | Useful for making plants with |
| | compared to wild-type. | modulated flowering time |
| SEQ_ID_NO_7166 | No seed is being produced by the plant | Useful for biocontainment applications |
| SEQ_ID_NO_7174 | The plant flowers significantly late | Useful for making plants with |
| | compared to wild-type. | modulated flowering time |
| SEQ_ID_NO_7174 | No seed is being produced by the plant | Useful for biocontainment applications |
| SEQ_ID_NO_7176 | | Useful for making plants with altered |
| | | cell wall content and/or composition |
| SEQ_ID_NO_72 | | Useful for making plants with altered |
| | | Lignin content |
| SEQ_ID_NO_72 | | Useful for making plants with altered |
| | | alkaloid content |
| SEQ_ID_NO_7216 | | Useful for making plants with |
| | | modulated phosphate use efficiency |
| SEQ_ID_NO_7218 | Useful for making plants with | The sequence is a functional homolog |
| | modulated growth and phenotype | of SEQ ID NO 837 in the 10/374,780 |
| | characteristics | US patent application |
| SEQ_ID_NO_7218 | Useful for making plants with | The sequence is a functional homolog |
| | modulated growth and phenotype | of SEQ ID NO 835 in the 10/374,780 |
| | characteristics | US patent application |
| SEQ_ID_NO_7218 | Useful for making plants with | The sequence is a functional homolog |
| | modulated growth and phenotype | of SEQ ID NO 712 in the 11/479,226 |
| | characteristics | US patent application |
| SEQ_ID_NO_7218 | Useful for making plants with | The sequence is a functional homolog |
| | modulated growth and phenotype | of SEQ ID NO 1190 in the 10/412,699 |
| | characteristics | US patent application |
| SEQ_ID_NO_7218 | Useful for making plants with | The sequence is a functional homolog |
| | modulated growth and phenotype | of SEQ ID NO 1188 in the 10/412,699 |
| | characteristics | US patent application |
| SEQ_ID_NO_7226 | The plant flowers significantly late | Useful for making plants with |
| | compared to wild-type. | modulated flowering time |
| SEQ_ID_NO_7226 | No seed is being produced by the plant | Useful for biocontainment applications |
| SEQ_ID_NO_7240 | | Useful for making plants with |
| | | modulated drought sensitivity |
| SEQ_ID_NO_7240 | Useful for making plants with | The sequence is a functional homolog |
| | modulated growth and phenotype | of SEQ ID NO 805 in the 11/479,226 |
| | characteristics | US patent application |
| SEQ_ID_NO_7240 | Useful for making plants with | The sequence is a functional homolog |
| | modulated growth and phenotype | of SEQ ID NO 801 in the 11/479,226 |
| | characteristics | US patent application |
| SEQ_ID_NO_7240 | Useful for making plants with | The sequence is a functional homolog |
| | modulated growth and phenotype | of SEQ ID NO 797 in the 11/479,226 |
| | characteristics | US patent application |
| SEQ_ID_NO_7240 | Useful for making plants with | The sequence is a functional homolog |
| | modulated growth and phenotype | of SEQ ID NO 790 in the 10/225,066 |
| | characteristics | US patent application |
| SEQ_ID_NO_7240 | Useful for making plants with | The sequence is a functional homolog |
| | modulated growth and phenotype | of SEQ ID NO 789 in the 11/479,226 |
| | characteristics | US patent application |
| SEQ_ID_NO_7240 | Useful for making plants with | The sequence is a functional homolog |
| | modulated growth and phenotype | of SEQ ID NO 786 in the 11/479,226 |
| | characteristics | US patent application |
| SEQ_ID_NO_7240 | Useful for making plants with | The sequence is a functional homolog |
| | modulated growth and phenotype | of SEQ ID NO 782 in the 11/479,226 |
| | characteristics | US patent application |
| SEQ_ID_NO_7240 | Useful for making plants with | The sequence is a functional homolog |
| | modulated growth and phenotype | of SEQ ID NO 344 in the 10/412,699 |
| | characteristics | US patent application |
| SEQ_ID_NO_7240 | Useful for making plants with | The sequence is a functional homolog |
| | modulated growth and phenotype | of SEQ ID NO 342 in the 10/412,699 |
| | characteristics | US patent application |
| SEQ_ID_NO_7240 | Useful for making plants with | The sequence is a functional homolog |
| | modulated growth and phenotype | of SEQ ID NO 2350 in the 10/374,780 |
| | characteristics | US patent application |
| SEQ_ID_NO_7250 | The plant produces a reduced number | Useful for biocontainment applications |
| | of seeds. | |
| SEQ_ID_NO_7252 | The plant flowers significantly earllier | Useful for making plants with |
| | than wild-type. | modulated flowering time |
| SEQ_ID_NO_7252 | | Useful for making plants with |
| | | modulated flowering time |
| SEQ_ID_NO_7252 | The plant is abnormally small. | Useful for making plants with |
| | | modulated biomass |
| SEQ_ID_NO_7252 | | Useful for making plants with |
| | | modulated biomass |
| SEQ_ID_NO_7258 | The plant flowers significantly late | Useful for making plants with |
| | compared to wild-type. | modulated flowering time |
| SEQ_ID_NO_7258 | No seed is being produced by the plant | Useful for biocontainment applications |
| SEQ_ID_NO_7276 | The plant is abnormally large. | Useful for making plants with |
| | | modulated biomass |
| SEQ_ID_NO_7276 | The plant is abnormally small. | Useful for making plants with |
| | | modulated biomass |
| SEQ_ID_NO_7286 | The plant is abnormally large. | Useful for making plants with |
| | | modulated biomass |
| SEQ_ID_NO_7300 | | Useful for making plants with altered |
| | | Lignin content |
| SEQ_ID_NO_7300 | | Useful for making plants with altered |
| | | cell wall content and/or composition |
| SEQ_ID_NO_7300 | | Useful for making plants with altered |
| | | cell wall composition |
| SEQ_ID_NO_7306 | | Useful for making plants with |
| | | modulated nitrogen use efficiency |
| SEQ_ID_NO_7308 | The plant is abnormally small. | Useful for making plants with |
| | | modulated biomass |
| SEQ_ID_NO_7308 | Useful for making plants with | The sequence is a functional homolog |
| | modulated growth and phenotype | of SEQ ID NO 960 in the 10/225,066 |
| | characteristics | US patent application |
| SEQ_ID_NO_7308 | Useful for making plants with | The sequence is a functional homolog |
| | modulated growth and phenotype | of SEQ ID NO 874 in the 10/412,699 |
| | characteristics | US patent application |
| SEQ_ID_NO_7308 | Useful for making plants with | The sequence is a functional homolog |
| | modulated growth and phenotype | of SEQ ID NO 2604 in the 10/374,780 |
| | characteristics | US patent application |
| SEQ_ID_NO_7308 | Useful for making plants with | The sequence is a functional homolog |
| | modulated growth and phenotype | of SEQ ID NO 1769 in the 11/479,226 |
| | characteristics | US patent application |
| SEQ_ID_NO_7308 | Useful for making plants with | The sequence is a functional homolog |
| | modulated growth and phenotype | of SEQ ID NO 1765 in the 11/479,226 |
| | characteristics | US patent application |
| SEQ_ID_NO_7316 | The plant is abnormally large. | Useful for making plants with |
| | | modulated biomass |
| SEQ_ID_NO_7316 | The plant is abnormally small. | Useful for making plants with |
| | | modulated biomass |
| SEQ_ID_NO_7350 | The plant flowers significantly late | Useful for making plants with |
| | compared to wild-type. | modulated flowering time |
| SEQ_ID_NO_7350 | No seed is being produced by the plant | Useful for biocontainment applications |
| SEQ_ID_NO_7352 | | Useful for making plants with |
| | | modulated heat sensitivity |
| SEQ_ID_NO_7352 | | Useful for making plants with altered |
| | | Lignin content |
| SEQ_ID_NO_7374 | | Useful for making plants with |
| | | modulated nitrogen use efficiency |
| SEQ_ID_NO_7462 | The plant flowers significantly late | Useful for making plants with |
| | compared to wild-type. | modulated flowering time |
| SEQ_ID_NO_7462 | No seed is being produced by the plant | Useful for biocontainment applications |
| SEQ_ID_NO_7466 | The plant produces a reduced number | Useful for biocontainment applications |
| | of seeds. | |
| SEQ_ID_NO_7480 | | Useful for making plants with |
| | | modulated oxidative stress sensitivity |
| SEQ_ID_NO_7480 | Useful for making plants with | The sequence is a functional homolog |
| | modulated growth and phenotype | of SEQ ID NO 464 in the 10/666,642 |
| | characteristics | US patent application |
| SEQ_ID_NO_7508 | | Useful for making plants with |
| | | modulated drought sensitivity |
| SEQ_ID_NO_7508 | Useful for making plants with | The sequence is a functional homolog |
| | modulated growth and phenotype | of SEQ ID NO 805 in the 11/479,226 |
| | characteristics | US patent application |
| SEQ_ID_NO_7508 | Useful for making plants with | The sequence is a functional homolog |
| | modulated growth and phenotype | of SEQ ID NO 801 in the 11/479,226 |
| | characteristics | US patent application |
| SEQ_ID_NO_7508 | Useful for making plants with | The sequence is a functional homolog |
| | modulated growth and phenotype | of SEQ ID NO 797 in the 11/479,226 |
| | characteristics | US patent application |
| SEQ_ID_NO_7508 | Useful for making plants with | The sequence is a functional homolog |
| | modulated growth and phenotype | of SEQ ID NO 790 in the 10/225,066 |
| | characteristics | US patent application |
| SEQ_ID_NO_7508 | Useful for making plants with | The sequence is a functional homolog |
| | modulated growth and phenotype | of SEQ ID NO 789 in the 11/479,226 |
| | characteristics | US patent application |
| SEQ_ID_NO_7508 | Useful for making plants with | The sequence is a functional homolog |
| | modulated growth and phenotype | of SEQ ID NO 786 in the 11/479,226 |
| | characteristics | US patent application |
| SEQ_ID_NO_7508 | Useful for making plants with | The sequence is a functional homolog |
| | modulated growth and phenotype | of SEQ ID NO 782 in the 11/479,226 |
| | characteristics | US patent application |
| SEQ_ID_NO_7508 | Useful for making plants with | The sequence is a functional homolog |
| | modulated growth and phenotype | of SEQ ID NO 344 in the 10/412,699 |
| | characteristics | US patent application |
| SEQ_ID_NO_7508 | Useful for making plants with | The sequence is a functional homolog |
| | modulated growth and phenotype | of SEQ ID NO 342 in the 10/412,699 |
| | characteristics | US patent application |
| SEQ_ID_NO_7508 | Useful for making plants with | The sequence is a functional homolog |
| | modulated growth and phenotype | of SEQ ID NO 2350 in the 10/374,780 |
| | characteristics | US patent application |
| SEQ_ID_NO_7516 | The plant produces a reduced number | Useful for biocontainment applications |
| | of seeds. | |
| SEQ_ID_NO_7520 | There is no branching. | Useful for making plants with |
| | | modulated biomass |
| SEQ_ID_NO_7532 | | Useful for making plants with |
| | | modulated phosphate use efficiency |
| SEQ_ID_NO_7532 | The plant flowers significantly late | Useful for making plants with |
| | compared to wild-type. | modulated flowering time |
| SEQ_ID_NO_7532 | The plant is abnormally large. | Useful for making plants with |
| | | modulated biomass |
| SEQ_ID_NO_7554 | The plant senesces significantly late | Useful for making plants with |
| | copared to wild-type. | modulated time to senescence |
| SEQ_ID_NO_7566 | The plant flowers significantly late | Useful for making plants with |
| | compared to wild-type. | modulated flowering time |
| SEQ_ID_NO_7566 | No seed is being produced by the plant | Useful for biocontainment applications |
| SEQ_ID_NO_7568 | The plant flowers significantly late | Useful for making plants with |
| | compared to wild-type. | modulated flowering time |
| SEQ_ID_NO_7568 | No seed is being produced by the plant | Useful for biocontainment applications |
| SEQ_ID_NO_7580 | The plant flowers significantly late | Useful for making plants with |
| | compared to wild-type. | modulated flowering time |
| SEQ_ID_NO_7580 | The plant is abnormally small. | Useful for making plants with |
| | | modulated biomass |
| SEQ_ID_NO_7596 | | Useful for making plants with |
| | | modulated phosphate use efficiency |
| SEQ_ID_NO_7596 | The plant flowers significantly late | Useful for making plants with |
| | compared to wild-type. | modulated flowering time |
| SEQ_ID_NO_7596 | The plant is abnormally large. | Useful for making plants with |
| | | modulated biomass |
| SEQ_ID_NO_7610 | | Useful for making plants with altered |
| | | Lignin content |
| SEQ_ID_NO_7610 | | Useful for making plants with altered |
| | | alkaloid content |
| SEQ_ID_NO_7616 | | Useful for making plants with altered |
| | | Lignin content |
| SEQ_ID_NO_7616 | | Useful for making plants with altered |
| | | alkaloid content |
| SEQ_ID_NO_7616 | Useful for making plants with | The sequence is a functional homolog |
| | modulated growth and phenotype | of SEQ ID NO 926 in the 10/225,066 |
| | characteristics | US patent application |
| SEQ_ID_NO_7616 | Useful for making plants with | The sequence is a functional homolog |
| | modulated growth and phenotype | of SEQ ID NO 846 in the 10/225,066 |
| | characteristics | US patent application |
| SEQ_ID_NO_7616 | Useful for making plants with | The sequence is a functional homolog |
| | modulated growth and phenotype | of SEQ ID NO 712 in the 11/479,226 |
| | characteristics | US patent application |
| SEQ_ID_NO_7616 | Useful for making plants with | The sequence is a functional homolog |
| | modulated growth and phenotype | of SEQ ID NO 314 in the 10/374,780 |
| | characteristics | US patent application |
| SEQ_ID_NO_7616 | Useful for making plants with | The sequence is a functional homolog |
| | modulated growth and phenotype | of SEQ ID NO 2126 in the 10/374,780 |
| | characteristics | US patent application |
| SEQ_ID_NO_7616 | Useful for making plants with | The sequence is a functional homolog |
| | modulated growth and phenotype | of SEQ ID NO 1908 in the 11/479,226 |
| | characteristics | US patent application |
| SEQ_ID_NO_7616 | Useful for making plants with | The sequence is a functional homolog |
| | modulated growth and phenotype | of SEQ ID NO 150 in the 10/374,780 |
| | characteristics | US patent application |
| SEQ_ID_NO_762 | The plant flowers significantly late | Useful for making plants with |
| | compared to wild-type. | modulated flowering time |
| SEQ_ID_NO_7628 | The plant senesces significantly late | Useful for making plants with |
| | copared to wild-type. | modulated time to senescence |
| SEQ_ID_NO_764 | | Useful for making plants with altered |
| | | terpenoid content |
| SEQ_ID_NO_7640 | | Useful for making plants with |
| | | modulated cold sensitivity |
| SEQ_ID_NO_7660 | | Useful for making plants with |
| | | modulated nitrogen use efficiency |
| SEQ_ID_NO_7668 | The plant produces a reduced number | Useful for biocontainment applications |
| | of seeds. | |
| SEQ_ID_NO_7692 | The plant is abnormally small. | Useful for making plants with |
| | | modulated biomass |
| SEQ_ID_NO_7700 | The plant flowers significantly late | Useful for making plants with |
| | compared to wild-type. | modulated flowering time |
| SEQ_ID_NO_7700 | No seed is being produced by the plant | Useful for biocontainment applications |
| SEQ_ID_NO_7706 | The plant is abnormally small. | Useful for making plants with |
| | | modulated biomass |
| SEQ_ID_NO_7728 | The plant produces a reduced number | Useful for biocontainment applications |
| | of seeds. | |
| SEQ_ID_NO_7730 | | Useful for making plants with |
| | | modulated drought sensitivity |
| SEQ_ID_NO_7730 | Useful for making plants with | The sequence is a functional homolog |
| | modulated growth and phenotype | of SEQ ID NO 805 in the 11/479,226 |
| | characteristics | US patent application |
| SEQ_ID_NO_7730 | Useful for making plants with | The sequence is a functional homolog |
| | modulated growth and phenotype | of SEQ ID NO 801 in the 11/479,226 |
| | characteristics | US patent application |
| SEQ_ID_NO_7730 | Useful for making plants with | The sequence is a functional homolog |
| | modulated growth and phenotype | of SEQ ID NO 797 in the 11/479,226 |
| | characteristics | US patent application |
| SEQ_ID_NO_7730 | Useful for making plants with | The sequence is a functional homolog |
| | modulated growth and phenotype | of SEQ ID NO 790 in the 10/225,066 |
| | characteristics | US patent application |
| SEQ_ID_NO_7730 | Useful for making plants with | The sequence is a functional homolog |
| | modulated growth and phenotype | of SEQ ID NO 789 in the 11/479,226 |
| | characteristics | US patent application |
| SEQ_ID_NO_7730 | Useful for making plants with | The sequence is a functional homolog |
| | modulated growth and phenotype | of SEQ ID NO 786 in the 11/479,226 |
| | characteristics | US patent application |
| SEQ_ID_NO_7730 | Useful for making plants with | The sequence is a functional homolog |
| | modulated growth and phenotype | of SEQ ID NO 782 in the 11/479,226 |
| | characteristics | US patent application |
| SEQ_ID_NO_7730 | Useful for making plants with | The sequence is a functional homolog |
| | modulated growth and phenotype | of SEQ ID NO 344 in the 10/412,699 |
| | characteristics | US patent application |
| SEQ_ID_NO_7730 | Useful for making plants with | The sequence is a functional homolog |
| | modulated growth and phenotype | of SEQ ID NO 342 in the 10/412,699 |
| | characteristics | US patent application |
| SEQ_ID_NO_7730 | Useful for making plants with | The sequence is a functional homolog |
| | modulated growth and phenotype | of SEQ ID NO 2350 in the 10/374,780 |
| | characteristics | US patent application |
| SEQ_ID_NO_7730 | Useful for making plants with | The sequence is a functional homolog |
| | modulated growth and phenotype | of SEQ ID NO 1777 in the 11/479,226 |
| | characteristics | US patent application |
| SEQ_ID_NO_7740 | | Useful for making plants with altered |
| | | Lignin content |
| SEQ_ID_NO_7740 | | Useful for making plants with altered |
| | | alkaloid content |
| SEQ_ID_NO_7746 | The plant senesces significantly late | Useful for making plants with |
| | copared to wild-type. | modulated time to senescence |
| SEQ_ID_NO_7764 | The plant flowers significantly late | Useful for making plants with |
| | compared to wild-type. | modulated flowering time |
| SEQ_ID_NO_7764 | The plant is abnormally small. | Useful for making plants with |
| | | modulated biomass |
| SEQ_ID_NO_7766 | | Useful for making plants with altered oil |
| | | content |
| SEQ_ID_NO_7772 | The plant flowers significantly late | Useful for making plants with |
| | compared to wild-type. | modulated flowering time |
| SEQ_ID_NO_7772 | No seed is being produced by the plant | Useful for biocontainment applications |
| SEQ_ID_NO_778 | | Useful for making plants with altered |
| | | Lignin content |
| SEQ_ID_NO_778 | | Useful for making plants with altered |
| | | alkaloid content |
| SEQ_ID_NO_7780 | The plant flowers significantly late | Useful for making plants with |
| | compared to wild-type. | modulated flowering time |
| SEQ_ID_NO_7780 | No seed is being produced by the plant | Useful for biocontainment applications |
| SEQ_ID_NO_782 | The plant is abnormally small. | Useful for making plants with |
| | | modulated biomass |
| SEQ_ID_NO_7834 | | Useful for making plants with |
| | | modulated nitrogen use efficiency |
| SEQ_ID_NO_7846 | The plant flowers significantly late | Useful for making plants with |
| | compared to wild-type. | modulated flowering time |
| SEQ_ID_NO_7846 | No seed is being produced by the plant | Useful for biocontainment applications |
| SEQ_ID_NO_7854 | The plant is abnormally large. | Useful for making plants with |
| | | modulated biomass |
| SEQ_ID_NO_7854 | The plant is abnormally small. | Useful for making plants with |
| | | modulated biomass |
| SEQ_ID_NO_7866 | | Useful for making plants with altered |
| | | Lignin content |
| SEQ_ID_NO_7866 | | Useful for making plants with altered |
| | | alkaloid content |
| SEQ_ID_NO_7874 | The plant is abnormally small. | Useful for making plants with |
| | | modulated biomass |
| SEQ_ID_NO_7876 | The plant flowers significantly late | Useful for making plants with |
| | compared to wild-type. | modulated flowering time |
| SEQ_ID_NO_7876 | No seed is being produced by the plant | Useful for biocontainment applications |
| SEQ_ID_NO_7878 | | Useful for making plants with |
| | | modulated nitrogen use efficiency |
| SEQ_ID_NO_7878 | The plant flowers significantly late | Useful for making plants with |
| | compared to wild-type. | modulated flowering time |
| SEQ_ID_NO_7878 | The plant is abnormally small. | Useful for making plants with |
| | | modulated biomass |
| SEQ_ID_NO_7878 | | Useful for making plants with altered |
| | | Lignin content |
| SEQ_ID_NO_7878 | | Useful for making plants with altered |
| | | alkaloid content |
| SEQ_ID_NO_7878 | Useful for making plants with | The sequence is a functional homolog |
| | modulated growth and phenotype | of SEQ ID NO 1016 in the 11/435,388 |
| | characteristics | US patent application |
| SEQ_ID_NO_7884 | The plant flowers significantly late | Useful for making plants with |
| | compared to wild-type. | modulated flowering time |
| SEQ_ID_NO_7884 | No seed is being produced by the plant | Useful for biocontainment applications |
| SEQ_ID_NO_7886 | Useful for making plants with | The sequence is a functional homolog |
| | modulated growth and phenotype | of SEQ ID NO 992 in the 11/479,226 |
| | characteristics | US patent application |
| SEQ_ID_NO_7886 | Useful for making plants with | The sequence is a functional homolog |
| | modulated growth and phenotype | of SEQ ID NO 988 in the 11/479,226 |
| | characteristics | US patent application |
| SEQ_ID_NO_7886 | Useful for making plants with | The sequence is a functional homolog |
| | modulated growth and phenotype | of SEQ ID NO 616 in the 11/435,388 |
| | characteristics | US patent application |
| SEQ_ID_NO_7886 | Useful for making plants with | The sequence is a functional homolog |
| | modulated growth and phenotype | of SEQ ID NO 504 in the 11/435,388 |
| | characteristics | US patent application |
| SEQ_ID_NO_7886 | Useful for making plants with | The sequence is a functional homolog |
| | modulated growth and phenotype | of SEQ ID NO 428 in the 10/412,699 |
| | characteristics | US patent application |
| SEQ_ID_NO_7908 | | Useful for making plants with |
| | | modulated cold sensitivity |
| SEQ_ID_NO_7936 | | Useful for making plants with |
| | | modulated nitrogen use efficiency |
| SEQ_ID_NO_7960 | | Useful for making plants with |
| | | modulated phosphate use efficiency |
| SEQ_ID_NO_7974 | The plant flowers significantly late | Useful for making plants with |
| | compared to wild-type. | modulated flowering time |
| SEQ_ID_NO_7974 | No seed is being produced by the plant | Useful for biocontainment applications |
| SEQ_ID_NO_8006 | | Useful for making plants with |
| | | modulated drought sensitivity |
| SEQ_ID_NO_8008 | The plant flowers significantly earllier | Useful for making plants with |
| | than wild-type. | modulated flowering time |
| SEQ_ID_NO_8008 | The plant is abnormally small. | Useful for making plants with |
| | | modulated biomass |
| SEQ_ID_NO_8008 | The plant produces a reduced number | Useful for biocontainment applications |
| | of seeds. | |
| SEQ_ID_NO_8056 | | Useful for making plants with |
| | | modulated phosphate use efficiency |
| SEQ_ID_NO_8056 | The plant flowers significantly late | Useful for making plants with |
| | compared to wild-type. | modulated flowering time |
| SEQ_ID_NO_8056 | The plant is abnormally large. | Useful for making plants with |
| | | modulated biomass |
| SEQ_ID_NO_806 | | Useful for making plants with |
| | | modulated cold sensitivity |
| SEQ_ID_NO_8060 | | Useful for making plants with |
| | | modulated drought sensitivity |
| SEQ_ID_NO_8060 | Useful for making plants with | The sequence is a functional homolog |
| | modulated growth and phenotype | of SEQ ID NO 805 in the 11/479,226 |
| | characteristics | US patent application |
| SEQ_ID_NO_8060 | Useful for making plants with | The sequence is a functional homolog |
| | modulated growth and phenotype | of SEQ ID NO 801 in the 11/479,226 |
| | characteristics | US patent application |
| SEQ_ID_NO_8060 | Useful for making plants with | The sequence is a functional homolog |
| | modulated growth and phenotype | of SEQ ID NO 797 in the 11/479,226 |
| | characteristics | US patent application |
| SEQ_ID_NO_8060 | Useful for making plants with | The sequence is a functional homolog |
| | modulated growth and phenotype | of SEQ ID NO 790 in the 10/225,066 |
| | characteristics | US patent application |
| SEQ_ID_NO_8060 | Useful for making plants with | The sequence is a functional homolog |
| | modulated growth and phenotype | of SEQ ID NO 789 in the 11/479,226 |
| | characteristics | US patent application |
| SEQ_ID_NO_8060 | Useful for making plants with | The sequence is a functional homolog |
| | modulated growth and phenotype | of SEQ ID NO 786 in the 11/479,226 |
| | characteristics | US patent application |
| SEQ_ID_NO_8060 | Useful for making plants with | The sequence is a functional homolog |
| | modulated growth and phenotype | of SEQ ID NO 782 in the 11/479,226 |
| | characteristics | US patent application |
| SEQ_ID_NO_8060 | Useful for making plants with | The sequence is a functional homolog |
| | modulated growth and phenotype | of SEQ ID NO 344 in the 10/412,699 |
| | characteristics | US patent application |
| SEQ_ID_NO_8060 | Useful for making plants with | The sequence is a functional homolog |
| | modulated growth and phenotype | of SEQ ID NO 342 in the 10/412,699 |
| | characteristics | US patent application |
| SEQ_ID_NO_8060 | Useful for making plants with | The sequence is a functional homolog |
| | modulated growth and phenotype | of SEQ ID NO 2350 in the 10/374,780 |
| | characteristics | US patent application |
| SEQ_ID_NO_8060 | Useful for making plants with | The sequence is a functional homolog |
| | modulated growth and phenotype | of SEQ ID NO 1777 in the 11/479,226 |
| | characteristics | US patent application |
| SEQ_ID_NO_8062 | The plant flowers significantly late | Useful for making plants with |
| | compared to wild-type. | modulated flowering time |
| SEQ_ID_NO_8086 | Useful for making plants with | The sequence is a functional homolog |
| | modulated growth and phenotype | of SEQ ID NO 960 in the 10/225,066 |
| | characteristics | US patent application |
| SEQ_ID_NO_8086 | Useful for making plants with | The sequence is a functional homolog |
| | modulated growth and phenotype | of SEQ ID NO 2604 in the 10/374,780 |
| | characteristics | US patent application |
| SEQ_ID_NO_8086 | Useful for making plants with | The sequence is a functional homolog |
| | modulated growth and phenotype | of SEQ ID NO 1769 in the 11/479,226 |
| | characteristics | US patent application |
| SEQ_ID_NO_8120 | | Useful for making plants with |
| | | modulated cold sensitivity |
| SEQ_ID_NO_8202 | | Useful for making plants with altered |
| | | cell wall content and/or composition |
| SEQ_ID_NO_8224 | | Useful for making plants with |
| | | modulated nitrogen use efficiency |
| SEQ_ID_NO_8240 | | Useful for making plants with |
| | | modulated nitrogen use efficiency |
| SEQ_ID_NO_8240 | The plant is abnormally small. | Useful for making plants with |
| | | modulated biomass |
| SEQ_ID_NO_8240 | | Useful for making plants with altered |
| | | Lignin content |
| SEQ_ID_NO_8240 | | Useful for making plants with altered |
| | | alkaloid content |
| SEQ_ID_NO_8240 | Useful for making plants with | The sequence is a functional homolog |
| | modulated growth and phenotype | of SEQ ID NO 1016 in the 11/435,388 |
| | characteristics | US patent application |
| SEQ_ID_NO_8256 | | Useful for making plants with |
| | | modulated salt sensitivity |
| SEQ_ID_NO_8258 | | Useful for making plants with |
| | | modulated phosphate use efficiency |
| SEQ_ID_NO_8258 | The plant flowers significantly late | Useful for making plants with |
| | compared to wild-type. | modulated flowering time |
| SEQ_ID_NO_8258 | The plant is abnormally large. | Useful for making plants with |
| | | modulated biomass |
| SEQ_ID_NO_8316 | | Useful for making plants with |
| | | modulated phosphate use efficiency |
| SEQ_ID_NO_8366 | The plant is abnormally small. | Useful for making plants with |
| | | modulated biomass |
| SEQ_ID_NO_8370 | The plant senesces significantly late | Useful for making plants with |
| | copared to wild-type. | modulated time to senescence |
| SEQ_ID_NO_8374 | The plant flowers significantly late | Useful for making plants with |
| | compared to wild-type. | modulated flowering time |
| SEQ_ID_NO_8374 | No seed is being produced by the plant | Useful for biocontainment applications |
| SEQ_ID_NO_8410 | The plant senesces significantly late | Useful for making plants with |
| | copared to wild-type. | modulated time to senescence |
| SEQ_ID_NO_8432 | The plant flowers significantly late | Useful for making plants with |
| | compared to wild-type. | modulated flowering time |
| SEQ_ID_NO_8432 | No seed is being produced by the plant | Useful for biocontainment applications |
| SEQ_ID_NO_8550 | | Useful for making plants with altered oil |
| | | content |
| SEQ_ID_NO_8552 | | Useful for making plants with |
| | | modulated drought sensitivity |
| SEQ_ID_NO_8552 | Useful for making plants with | The sequence is a functional homolog |
| | modulated growth and phenotype | of SEQ ID NO 805 in the 11/479,226 |
| | characteristics | US patent application |
| SEQ_ID_NO_8552 | Useful for making plants with | The sequence is a functional homolog |
| | modulated growth and phenotype | of SEQ ID NO 801 in the 11/479,226 |
| | characteristics | US patent application |
| SEQ_ID_NO_8552 | Useful for making plants with | The sequence is a functional homolog |
| | modulated growth and phenotype | of SEQ ID NO 797 in the 11/479,226 |
| | characteristics | US patent application |
| SEQ_ID_NO_8552 | Useful for making plants with | The sequence is a functional homolog |
| | modulated growth and phenotype | of SEQ ID NO 790 in the 10/225,066 |
| | characteristics | US patent application |
| SEQ_ID_NO_8552 | Useful for making plants with | The sequence is a functional homolog |
| | modulated growth and phenotype | of SEQ ID NO 789 in the 11/479,226 |
| | characteristics | US patent application |
| SEQ_ID_NO_8552 | Useful for making plants with | The sequence is a functional homolog |
| | modulated growth and phenotype | of SEQ ID NO 786 in the 11/479,226 |
| | characteristics | US patent application |
| SEQ_ID_NO_8552 | Useful for making plants with | The sequence is a functional homolog |
| | modulated growth and phenotype | of SEQ ID NO 782 in the 11/479,226 |
| | characteristics | US patent application |
| SEQ_ID_NO_8552 | Useful for making plants with | The sequence is a functional homolog |
| | modulated growth and phenotype | of SEQ ID NO 344 in the 10/412,699 |
| | characteristics | US patent application |
| SEQ_ID_NO_8552 | Useful for making plants with | The sequence is a functional homolog |
| | modulated growth and phenotype | of SEQ ID NO 342 in the 10/412,699 |
| | characteristics | US patent application |
| SEQ_ID_NO_8552 | Useful for making plants with | The sequence is a functional homolog |
| | modulated growth and phenotype | of SEQ ID NO 2350 in the 10/374,780 |
| | characteristics | US patent application |
| SEQ_ID_NO_8552 | Useful for making plants with | The sequence is a functional homolog |
| | modulated growth and phenotype | of SEQ ID NO 1777 in the 11/479,226 |
| | characteristics | US patent application |
| SEQ_ID_NO_8638 | | Useful for making plants with altered |
| | | Lignin content |
| SEQ_ID_NO_8638 | | Useful for making plants with altered |
| | | cell wall content and/or composition |
| SEQ_ID_NO_8654 | The plant is abnormally large. | Useful for making plants with |
| | | modulated biomass |
| SEQ_ID_NO_8658 | | Useful for making plants with |
| | | modulated nitrogen use efficiency |
| SEQ_ID_NO_866 | | Useful for making plants with |
| | | modulated phosphate use efficiency |
| SEQ_ID_NO_866 | The plant flowers significantly late | Useful for making plants with |
| | compared to wild-type. | modulated flowering time |
| SEQ_ID_NO_866 | The plant is abnormally large. | Useful for making plants with |
| | | modulated biomass |
| SEQ_ID_NO_8680 | | Useful for making plants with altered |
| | | Lignin content |
| SEQ_ID_NO_8700 | The plant flowers significantly late | Useful for making plants with |
| | compared to wild-type. | modulated flowering time |
| SEQ_ID_NO_8700 | No seed is being produced by the plant | Useful for biocontainment applications |
| SEQ_ID_NO_872 | The plant is abnormally large. | Useful for making plants with |
| | | modulated biomass |
| SEQ_ID_NO_872 | Useful for making plants with | The sequence is a functional homolog |
| | modulated growth and phenotype | of SEQ ID NO 786 in the 10/412,699 |
| | characteristics | US patent application |
| SEQ_ID_NO_872 | Useful for making plants with | The sequence is a functional homolog |
| | modulated growth and phenotype | of SEQ ID NO 784 in the 10/412,699 |
| | characteristics | US patent application |
| SEQ_ID_NO_872 | Useful for making plants with | The sequence is a functional homolog |
| | modulated growth and phenotype | of SEQ ID NO 1617 in the 11/479,226 |
| | characteristics | US patent application |
| SEQ_ID_NO_872 | Useful for making plants with | The sequence is a functional homolog |
| | modulated growth and phenotype | of SEQ ID NO 1614 in the 11/479,226 |
| | characteristics | US patent application |
| SEQ_ID_NO_8726 | | Useful for making plants with altered |
| | | alkaloid content |
| SEQ_ID_NO_8736 | The plant is abnormally large. | Useful for making plants with |
| | | modulated biomass |
| SEQ_ID_NO_8736 | Useful for making plants with | The sequence is a functional homolog |
| | modulated growth and phenotype | of SEQ ID NO 786 in the 10/412,699 |
| | characteristics | US patent application |
| SEQ_ID_NO_8736 | Useful for making plants with | The sequence is a functional homolog |
| | modulated growth and phenotype | of SEQ ID NO 784 in the 10/412,699 |
| | characteristics | US patent application |
| SEQ_ID_NO_8736 | Useful for making plants with | The sequence is a functional homolog |
| | modulated growth and phenotype | of SEQ ID NO 1617 in the 11/479,226 |
| | characteristics | US patent application |
| SEQ_ID_NO_8736 | Useful for making plants with | The sequence is a functional homolog |
| | modulated growth and phenotype | of SEQ ID NO 1614 in the 11/479,226 |
| | characteristics | US patent application |
| SEQ_ID_NO_8746 | The plant flowers significantly earllier | Useful for making plants with |
| | than wild-type. | modulated flowering time |
| SEQ_ID_NO_8746 | | Useful for making plants with |
| | | modulated flowering time |
| SEQ_ID_NO_8746 | The plant is abnormally small. | Useful for making plants with |
| | | modulated biomass |
| SEQ_ID_NO_8746 | | Useful for making plants with |
| | | modulated biomass |
| SEQ_ID_NO_8758 | The plant flowers significantly late | Useful for making plants with |
| | compared to wild-type. | modulated flowering time |
| SEQ_ID_NO_8758 | No seed is being produced by the plant | Useful for biocontainment applications |
| SEQ_ID_NO_8780 | | Useful for making plants with altered |
| | | cell wall content and/or composition |
| SEQ_ID_NO_8800 | | Useful for making plants with altered oil |
| | | content |
| SEQ_ID_NO_8802 | | Useful for making plants with altered |
| | | alkaloid content |
| SEQ_ID_NO_8828 | | Useful for making plants with altered |
| | | terpenoid content |
| SEQ_ID_NO_8828 | | Useful for making plants with altered |
| | | Lignin content |
| SEQ_ID_NO_8912 | | Useful for making plants with |
| | | modulated nitrogen use efficiency |
| SEQ_ID_NO_8912 | The plant flowers significantly late | Useful for making plants with |
| | compared to wild-type. | modulated flowering time |
| SEQ_ID_NO_8912 | | Useful for making plants with altered |
| | | Lignin content |
| SEQ_ID_NO_8912 | | Useful for making plants with altered |
| | | alkaloid content |
| SEQ_ID_NO_8912 | Useful for making plants with | The sequence is a functional homolog |
| | modulated growth and phenotype | of SEQ ID NO 1016 in the 11/435,388 |
| | characteristics | US patent application |
| SEQ_ID_NO_8934 | | Useful for making plants with altered |
| | | Lignin content |
| SEQ_ID_NO_8934 | | Useful for making plants with altered |
| | | alkaloid content |
| SEQ_ID_NO_8942 | | Useful for making plants with |
| | | modulated phosphate use efficiency |
| SEQ_ID_NO_8968 | Useful for making plants with | The sequence is a functional homolog |
| | modulated growth and phenotype | of SEQ ID NO 898 in the 10/666,642 |
| | characteristics | US patent application |
| SEQ_ID_NO_8968 | Useful for making plants with | The sequence is a functional homolog |
| | modulated growth and phenotype | of SEQ ID NO 1423 in the 10/412,699 |
| | characteristics | US patent application |
| SEQ_ID_NO_8968 | Useful for making plants with | The sequence is a functional homolog |
| | modulated growth and phenotype | of SEQ ID NO 1270 in the 10/374,780 |
| | characteristics | US patent application |
| SEQ_ID_NO_8996 | Useful for making plants with | The sequence is a functional homolog |
| | modulated growth and phenotype | of SEQ ID NO 990 in the 10/412,699 |
| | characteristics | US patent application |
| SEQ_ID_NO_8996 | Useful for making plants with | The sequence is a functional homolog |
| | modulated growth and phenotype | of SEQ ID NO 988 in the 10/412,699 |
| | characteristics | US patent application |
| SEQ_ID_NO_8996 | Useful for making plants with | The sequence is a functional homolog |
| | modulated growth and phenotype | of SEQ ID NO 806 in the 11/435,388 |
| | characteristics | US patent application |
| SEQ_ID_NO_8996 | Useful for making plants with | The sequence is a functional homolog |
| | modulated growth and phenotype | of SEQ ID NO 683 in the 10/666,642 |
| | characteristics | US patent application |
| SEQ_ID_NO_8996 | Useful for making plants with | The sequence is a functional homolog |
| | modulated growth and phenotype | of SEQ ID NO 681 in the 10/666,642 |
| | characteristics | US patent application |
| SEQ_ID_NO_8996 | Useful for making plants with | The sequence is a functional homolog |
| | modulated growth and phenotype | of SEQ ID NO 490 in the 10/374,780 |
| | characteristics | US patent application |
| SEQ_ID_NO_8996 | Useful for making plants with | The sequence is a functional homolog |
| | modulated growth and phenotype | of SEQ ID NO 488 in the 10/374,780 |
| | characteristics | US patent application |
| SEQ_ID_NO_8996 | Useful for making plants with | The sequence is a functional homolog |
| | modulated growth and phenotype | of SEQ ID NO 198 in the 11/375,241 |
| | characteristics | US patent application |
| SEQ_ID_NO_9042 | | Useful for making plants with altered oil |
| | | content |
| SEQ_ID_NO_9060 | The plant flowers significantly late | Useful for making plants with |
| | compared to wild-type. | modulated flowering time |
| SEQ_ID_NO_9060 | No seed is being produced by the plant | Useful for biocontainment applications |
| SEQ_ID_NO_9062 | The plant flowers significantly late | Useful for making plants with |
| | compared to wild-type. | modulated flowering time |
| SEQ_ID_NO_9062 | | Useful for making plants with |
| | | modulated cold sensitivity |
| SEQ_ID_NO_9070 | | Useful for making plants with altered |
| | | cell wall content and/or composition |
| SEQ_ID_NO_9090 | | Useful for making plants with |
| | | modulated nitrogen use efficiency |
| SEQ_ID_NO_9098 | | Useful for making plants with |
| | | modulated flowering time |
| SEQ_ID_NO_9106 | The plant is abnormally large. | Useful for making plants with |
| | | modulated biomass |
| SEQ_ID_NO_9106 | The plant is abnormally small. | Useful for making plants with |
| | | modulated biomass |
| SEQ_ID_NO_9132 | The plant flowers significantly earllier | Useful for making plants with |
| | than wild-type. | modulated flowering time |
| SEQ_ID_NO_9132 | | Useful for making plants with |
| | | modulated flowering time |
| SEQ_ID_NO_9132 | The plant is abnormally small. | Useful for making plants with |
| | | modulated biomass |
| SEQ_ID_NO_9132 | | Useful for making plants with |
| | | modulated biomass |
| SEQ_ID_NO_9156 | | Useful for making plants with |
| | | modulated flowering time |
| SEQ_ID_NO_9174 | | Useful for making plants with altered oil |
| | | content |
| SEQ_ID_NO_9262 | The plant senesces significantly late | Useful for making plants with |
| | copared to wild-type. | modulated time to senescence |
| SEQ_ID_NO_9262 | The plant flowers significantly late | Useful for making plants with |
| | compared to wild-type. | modulated flowering time |
| SEQ_ID_NO_9322 | | Useful for making plants with altered |
| | | terpenoid content |
| SEQ_ID_NO_9322 | | Useful for making plants with altered |
| | | alkaloid content |
| SEQ_ID_NO_9374 | | Useful for making plants with |
| | | modulated phosphate use efficiency |
| SEQ_ID_NO_9374 | The plant flowers significantly late | Useful for making plants with |
| | compared to wild-type. | modulated flowering time |
| SEQ_ID_NO_9374 | The plant is abnormally large. | Useful for making plants with |
| | | modulated biomass |
| SEQ_ID_NO_9382 | | Useful for making plants with |
| | | modulated cold sensitivity |
| SEQ_ID_NO_9442 | Useful for making plants with | The sequence is a functional homolog |
| | modulated growth and phenotype | of SEQ ID NO 992 in the 11/479,226 |
| | characteristics | US patent application |
| SEQ_ID_NO_9442 | Useful for making plants with | The sequence is a functional homolog |
| | modulated growth and phenotype | of SEQ ID NO 988 in the 11/479,226 |
| | characteristics | US patent application |
| SEQ_ID_NO_9442 | Useful for making plants with | The sequence is a functional homolog |
| | modulated growth and phenotype | of SEQ ID NO 616 in the 11/435,388 |
| | characteristics | US patent application |
| SEQ_ID_NO_9442 | Useful for making plants with | The sequence is a functional homolog |
| | modulated growth and phenotype | of SEQ ID NO 504 in the 11/435,388 |
| | characteristics | US patent application |
| SEQ_ID_NO_9442 | Useful for making plants with | The sequence is a functional homolog |
| | modulated growth and phenotype | of SEQ ID NO 428 in the 10/412,699 |
| | characteristics | US patent application |
| SEQ_ID_NO_9502 | | Useful for making plants with |
| | | modulated drought sensitivity |
| SEQ_ID_NO_9502 | Useful for making plants with | The sequence is a functional homolog |
| | modulated growth and phenotype | of SEQ ID NO 805 in the 11/479,226 |
| | characteristics | US patent application |
| SEQ_ID_NO_9502 | Useful for making plants with | The sequence is a functional homolog |
| | modulated growth and phenotype | of SEQ ID NO 801 in the 11/479,226 |
| | characteristics | US patent application |
| SEQ_ID_NO_9502 | Useful for making plants with | The sequence is a functional homolog |
| | modulated growth and phenotype | of SEQ ID NO 797 in the 11/479,226 |
| | characteristics | US patent application |
| SEQ_ID_NO_9502 | Useful for making plants with | The sequence is a functional homolog |
| | modulated growth and phenotype | of SEQ ID NO 790 in the 10/225,066 |
| | characteristics | US patent application |
| SEQ_ID_NO_9502 | Useful for making plants with | The sequence is a functional homolog |
| | modulated growth and phenotype | of SEQ ID NO 789 in the 11/479,226 |
| | characteristics | US patent application |
| SEQ_ID_NO_9502 | Useful for making plants with | The sequence is a functional homolog |
| | modulated growth and phenotype | of SEQ ID NO 786 in the 11/479,226 |
| | characteristics | US patent application |
| SEQ_ID_NO_9502 | Useful for making plants with | The sequence is a functional homolog |
| | modulated growth and phenotype | of SEQ ID NO 782 in the 11/479,226 |
| | characteristics | US patent application |
| SEQ_ID_NO_9502 | Useful for making plants with | The sequence is a functional homolog |
| | modulated growth and phenotype | of SEQ ID NO 344 in the 10/412,699 |
| | characteristics | US patent application |
| SEQ_ID_NO_9502 | Useful for making plants with | The sequence is a functional homolog |
| | modulated growth and phenotype | of SEQ ID NO 342 in the 10/412,699 |
| | characteristics | US patent application |
| SEQ_ID_NO_9502 | Useful for making plants with | The sequence is a functional homolog |
| | modulated growth and phenotype | of SEQ ID NO 2350 in the 10/374,780 |
| | characteristics | US patent application |
| SEQ_ID_NO_9624 | | Useful for making plants with |
| | | modulated nitrogen use efficiency |
| SEQ_ID_NO_9624 | The plant flowers significantly late | Useful for making plants with |
| | compared to wild-type. | modulated flowering time |
| SEQ_ID_NO_9624 | | Useful for making plants with altered |
| | | Lignin content |
| SEQ_ID_NO_9624 | | Useful for making plants with altered |
| | | alkaloid content |
| SEQ_ID_NO_9624 | Useful for making plants with | The sequence is a functional homolog |
| | modulated growth and phenotype | of SEQ ID NO 1016 in the 11/435,388 |
| | characteristics | US patent application |
| SEQ_ID_NO_9636 | | Useful for making plants with altered |
| | | Lignin content |
| SEQ_ID_NO_9636 | | Useful for making plants with altered |
| | | cell wall content and/or composition |
| SEQ_ID_NO_9636 | | Useful for making plants with altered |
| | | cell wall composition |
| SEQ_ID_NO_9670 | Useful for making plants with | The sequence is a functional homolog |
| | modulated growth and phenotype | of SEQ ID NO 872 in the 10/666,642 |
| | characteristics | US patent application |
| SEQ_ID_NO_9670 | Useful for making plants with | The sequence is a functional homolog |
| | modulated growth and phenotype | of SEQ ID NO 1466 in the 10/374,780 |
| | characteristics | US patent application |
| SEQ_ID_NO_9704 | | Useful for making plants with |
| | | modulated nitrogen use efficiency |
| SEQ_ID_NO_9704 | The plant flowers significantly late | Useful for making plants with |
| | compared to wild-type. | modulated flowering time |
| SEQ_ID_NO_9704 | | Useful for making plants with altered |
| | | Lignin content |
| SEQ_ID_NO_9704 | | Useful for making plants with altered |
| | | alkaloid content |
| SEQ_ID_NO_9704 | Useful for making plants with | The sequence is a functional homolog |
| | modulated growth and phenotype | of SEQ ID NO 1016 in the 11/435,388 |
| | characteristics | US patent application |
| SEQ_ID_NO_9718 | The plant flowers significantly earllier | Useful for making plants with |
| | than wild-type. | modulated flowering time |
| SEQ_ID_NO_9718 | | Useful for making plants with |
| | | modulated flowering time |
| SEQ_ID_NO_9718 | The plant is abnormally small. | Useful for making plants with |
| | | modulated biomass |
| SEQ_ID_NO_9718 | | Useful for making plants with |
| | | modulated biomass |
| SEQ_ID_NO_9724 | | Useful for making plants with |
| | | modulated nitrogen use efficiency |
| SEQ_ID_NO_9724 | The plant flowers significantly late | Useful for making plants with |
| | compared to wild-type. | modulated flowering time |
| SEQ_ID_NO_9724 | | Useful for making plants with altered |
| | | Lignin content |
| SEQ_ID_NO_9724 | | Useful for making plants with altered |
| | | alkaloid content |
| SEQ_ID_NO_9724 | Useful for making plants with | The sequence is a functional homolog |
| | modulated growth and phenotype | of SEQ ID NO 1016 in the 11/435,388 |
| | characteristics | US patent application |
| SEQ_ID_NO_9752 | | Useful for making plants with |
| | | modulated oxidative stress sensitivity |
| SEQ_ID_NO_9764 | | Useful for making plants with altered |
| | | terpenoid content |
| SEQ_ID_NO_9788 | | Useful for making plants with altered |
| | | alkaloid content |
| SEQ_ID_NO_9798 | | Useful for making plants with altered |
| | | cell wall content and/or composition |
| SEQ_ID_NO_9808 | Useful for making plants with | The sequence is a functional homolog |
| | modulated growth and phenotype | of SEQ ID NO 891 in the 10/374,780 |
| | characteristics | US patent application |
| SEQ_ID_NO_9808 | Useful for making plants with | The sequence is a functional homolog |
| | modulated growth and phenotype | of SEQ ID NO 890 in the 10/374,780 |
| | characteristics | US patent application |
| SEQ_ID_NO_9808 | Useful for making plants with | The sequence is a functional homolog |
| | modulated growth and phenotype | of SEQ ID NO 886 in the 10/225,066 |
| | characteristics | US patent application |
| SEQ_ID_NO_9808 | Useful for making plants with | The sequence is a functional homolog |
| | modulated growth and phenotype | of SEQ ID NO 853 in the 10/374,780 |
| | characteristics | US patent application |
| SEQ_ID_NO_9808 | Useful for making plants with | The sequence is a functional homolog |
| | modulated growth and phenotype | of SEQ ID NO 731 in the 11/479,226 |
| | characteristics | US patent application |
| SEQ_ID_NO_9808 | Useful for making plants with | The sequence is a functional homolog |
| | modulated growth and phenotype | of SEQ ID NO 312 in the 10/412,699 |
| | characteristics | US patent application |
| SEQ_ID_NO_9808 | Useful for making plants with | The sequence is a functional homolog |
| | modulated growth and phenotype | of SEQ ID NO 2012 in the 10/374,780 |
| | characteristics | US patent application |
| SEQ_ID_NO_9808 | Useful for making plants with | The sequence is a functional homolog |
| | modulated growth and phenotype | of SEQ ID NO 102 in the 10/374,780 |
| | characteristics | US patent application |
| SEQ_ID_NO_982 | The plant senesces significantly late | Useful for making plants with |
| | copared to wild-type. | modulated time to senescence |
| SEQ_ID_NO_982 | The plant flowers significantly late | Useful for making plants with |
| | compared to wild-type. | modulated flowering time |
| SEQ_ID_NO_9840 | | Useful for making plants with altered |
| | | cell wall content and/or composition |
| SEQ_ID_NO_9904 | | Useful for making plants with altered |
| | | cell wall content and/or composition |
| SEQ_ID_NO_9906 | | Useful for making plants with altered |
| | | terpenoid content |
| SEQ_ID_NO_992 | | Useful for making plants with altered |
| | | cell wall content and/or composition |
| SEQ_ID_NO_994 | Useful for making plants with | The sequence is a functional homolog |
| | modulated growth and phenotype | of SEQ ID NO 960 in the 10/225,066 |
| | characteristics | US patent application |
| SEQ_ID_NO_994 | Useful for making plants with | The sequence is a functional homolog |
| | modulated growth and phenotype | of SEQ ID NO 2604 in the 10/374,780 |
| | characteristics | US patent application |
| SEQ_ID_NO_994 | Useful for making plants with | The sequence is a functional homolog |
| | modulated growth and phenotype | of SEQ ID NO 1769 in the 11/479,226 |
| | characteristics | US patent application |

### Biocontainment

While growing transgenic plants for agronomic purposes is becoming more common, concerns about preventing cross-pollination with naturally occurring plants remain. One approach to containing the germplasm of transgenic plants is patterned after that used for hybrid seed production. To produce hybrid seed that is not contaminated with selfed seed pollination, cross-pollination and not self-pollination is ensured, for example, by using a genetic method of pollination control. Here, plants that are used as females (i) fail to make pollen, (ii) fail to shed pollen, or (iii) produce pollen biochemically unable to provide self-fertilization. Alternatively, pollen control is based on plants that are male sterile due to (i) nuclear or genic male sterility—the failure of pollen formation because of, typically, mutations in one or more nuclear genes; or (ii) cytoplasmic male sterility (CMS)—pollen formation is blocked or aborted as the result of a defect in a cytoplasmic organelle, typically, the mitochondria. For biocontainment purposes, use of these types of semi-sterile plants allow germplasm containment in all types of plants, except that female plants of type (iii) above are only useful for germplasm containment in obligatory self-pollinating species.

Another genetic approach to biocontainment is to link the recombinant traits of interest to repressible lethal genes (for example see WO 00/37660). The lethal genes are blocked by the action of repressor molecules produced by repressor genes located at a different genetic locus. The lethal phenotype is expressed only if the repressible lethal gene construct and the repressor gene segregate after meiosis. This approach reportedly can be used to maintain genetic purity by blocking introgression of genes from plants that lack the repressor gene.

### Altered Oil Content

Increasing the oil content, or optimizing the oil composition, of plants has long been a goal in plant breeding programs. As plant oil production becomes better understood, the potential of using transgenic means to accomplish this has increased. One way to achieve increased oil content/composition is to manipulate the genes involved in oil production pathways. For example, acetyl CoA carboxylase (ACCase) is an enzyme that is especially important in fatty acid synthesis in plants and is sensitive to inhibition by some types of herbicides. Using acetyl-CoA and bicarbonate, Acetyl CoA Carboxylase (ACCase) catalyzes the formation of malonyl-CoA, an essential substrate for (i) de novo fatty acid (FA) synthesis, (ii) fatty acid elongation, (iii) synthesis of secondary metabolites such as flavonoids and anthocyanins, and (iv) malonylation of some amino acids and secondary metabolites.

In plants, most ACCase activity is located in plastids of green and non-green plant tissues including leaves and oil seeds. Condensation of malonyl-CoA with phenylpropionyl-CoAs or acetyl-CoA leads to synthesis of flavonoids, anthocyanins, or to polyacetates. In addition to the secondary metabolites derived by de novo synthesis, malonyl conjugates of flavonoid glycosides, formed by malonyl-CoA:flavonoid glycoside malonyltransferase, D-amino acids and 1-amino-carboxyl-cyclopropane (ethylene precursor) are found in plants. Malonylated compounds accumulate in vacuoles, probably after synthesis in the cytoplasm. Thus, manipulating any of the genes involved in this pathway can produce improvements in plant oil content as well as secondary metabolites such as anthocyanins.

### Herbicide Tolerance

There are three general mechanisms by which plants may be resistant to, or tolerant of, herbicides. These mechanisms include insensitivity at the site of action of the herbicide (usually an enzyme), rapid metabolism (conjugation or degradation) of the herbicide, or poor uptake and translocation of the herbicide. Altering the herbicide site of action from a sensitive to an insensitive form is the preferred method of conferring tolerance on a sensitive plant species. This is because tolerance of this nature is likely to be a dominant trait encoded by a single gene, and is likely to encompass whole families of compounds that share a single site of action, not just individual chemicals. Two examples of families of herbicides are the cyclohexanedione family and herbicidal aryloxyphenoxypropanoic acids. Therefore, detailed information concerning the biochemical site and mechanism of herbicide action is of great importance and can be applied in two ways. First, the genes involved in the production of the biochemical site and/or mechanism of action can be manipulated to produce resistance or enhance susceptibility. Second, the information can be used to develop cell selection strategies for the efficient identification and isolation of appropriate herbicide-tolerant/susceptible variants. Third, it can be used to characterize the variant cell lines and regenerated plants that result from the selections.

### Modulated Sterol Content

Sterols are known to play at least two critical roles in plants: as bulk components of membranes regulating stability and permeability (Bach et al. (1997) Prog. Lipid Res. 36:197 226) and as precursors of growth-promoting brassinosteroids (BRs; Fujioka and Sakurai (1997) Nat. Prod. Rep. 14:1 10). Sterol biosynthesis in plants has been studied extensively through enzyme purification or gene cloning (Grunwald (1975) Annu. Rev. Plant Physiol. 26:209 236; Goodwin (1979) Annu. Rev. Plant Physiol. 30:369 404; Benveniste (1986) Annu. Rev. Plant Physiol. 37:275 308; Bach and Benveniste (1997) Prog. Lipid Res. 36:197 226; all of which are incorporated by reference by in their entirety). A major difference between photosynthetic and nonphotosynthetic organisms is that cyclization of squalene 2,3-oxide is bifurcated to a different route for each system (Benveniste (1986) Annu. Rev. Plant Physiol. 37:275 308). Photosynthetic organisms require biosynthetic enzymes such as cycloartenol synthase (Corey et al. (1993) Proc. Natl. Acad. Sci. USA 90:11628 11632) and cycloeucalenol-obtusifoliol isomerase, which are required to open the cyclopropane ring in cycloartenol. Thus, manipulating the expression of genes in the sterol pathway can result in plants having altered sterol content and composition.

### Altered Alkaloid Content

Plant families that produce alkaloids include the Papaveraceae, Berberidaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, and Rutaceae families. Many alkaloids isolated from such plants are known for their pharmacologic (e.g., narcotic), insecticidal, and physiologic effects. For example, the poppy (Papaveraceae) family contains about 250 species found mainly in the northern temperate regions of the world. The principal morphinan alkaloids in opium poppy (Papaver somniferum) are morphine, codeine, and thebaine, which are used directly or modified using synthetic methods to produce pharmaceutical compounds used for pain management, cough suppression, and addiction. Manipulation of the genes participating in the alkaloid biosynthetic pathways not only allow production of plants with altered alkaloid content and/or composition, but also enable the production of new types of alkaloids for use in medicine and agriculture.

### Altered Carbon Content

The ability of a plant to grow and develop under diverse and changing environmental conditions depends on the ability of the plant to utilize carbon and/or nitrogen. Specifically, the accumulation of one or both of these elements suggests that the plant is storing, synthesizing, or utilizing components such as nitrate, amino acids, proteins, sugars and/or carbohydrates to compensate for the changing environment. The balance of carbon and nitrogen in plants is an important aspect of how plants utilize nitrogen efficiently. Carbon skeletons and energy are required in ample supply for nitrogen assimilation and re-assimilation (photorespiratory NH₄). Conversely, primary carbon assimilation is highly dependent on nitrogen assimilation because much of the nitrogen in a plant is invested in the proteins and chlorophyll of the photosynthetic machinery. Therefore, fixed carbon must be partitioned between amino acids and carbohydrate synthesis in a flexible manner that is responsive to the external and internal availability of nitrogen. Thus plant genes that increase fixed carbon content under varying nitrogen conditions are useful for optimizing carbon partitioning in plants.

### Altered Nitrogen Content

The photoautotrophic production of organic nitrogenous compounds is crucial to plant metabolism, growth, and development. Protein and amino acid contents of harvested plant materials are of great agronomic importance in many crop species. Light-driven nitrogen (N) assimilation in leaves has evolved to operate alongside and integrate with photosynthesis and respiration. The production of reduced carbon (C) in photosynthesis and its reoxidation in respiration are necessary to produce both the energy and C skeletons required for the incorporation of inorganic N into amino acids. Conversely, N assimilation is required to sustain the output of organic C and N. This network is further complicated by the concomitant operation of photorespiratory metabolism. Both the rate of N assimilation and the coordination of C and N assimilation are under multifactorial control by a repertoire of signals, which provide information on C and N status. Consequently, genes that increase nitrogen content in plants under varying nitrogen conditions are useful in optimizing the nitrogen available for protein and amino acid synthesis.

### Altered Sugar Content

A sugar is a carbohydrate that is sweet to taste. Sugars are used in food and drink as a source of sweetness and energy and are important in biochemistry. Sucrose, also called “table sugar,” is a white crystalline solid. Sucrose is a disaccharide composed of two monosaccharides, glucose and fructose, joined together by a 1→2, α, β-glycosidic bond. Sucrose is commercially extracted from either sugar cane or sugar beet and then purified and crystallized. Other commercial sources are sorghum, date palm, and sugar maples. The monosaccharides, such as glucose (which is produced from sucrose by enzymes or acid hydrolysis), are a store of energy that is used by biological cells. Oxidation of glucose is known as glycolysis. It occurs in virtually all cells. Glucose is oxidized to either lactate or pyruvate. Under aerobic conditions, the dominant product in most tissues is pyruvate and the pathway is known as aerobic glycolysis. When oxygen is depleted, as for instance during prolonged vigorous exercise, the dominant glycolytic product in many tissues is lactate and the process is known as anaerobic glycolysis. Other sugars besides glucose, such as fructose, can enter glycolysis after being converted to appropriate intermediates that can enter the pathway. Glycolysis results in production of NADH and ATP. The NADH generated during glycolysis is used to fuel mitochondrial ATP synthesis via oxidative phosphorylation. ATP powers virtually every activity of the cell and organism. Organisms from the simplest bacteria to humans use ATP as their primary energy currency. Thus, manipulation of the genes used in these biochemical pathways and for these enzymes allow production of plants with modulated (i.e. increased or decreased) sugar content.

### Altered Amino Acid Content

An essential amino acid for an organism is an amino acid that cannot be synthesized by the organism from other available resources, and therefore must be supplied as part of its diet. Nine amino acids, including lysine and leucine, are generally regarded as essential for humans. Deficiencies of particular essential amino acids in certain major food crops have spurred efforts to improve the nutritional value of plants. One strategy to improve the nutritional value of plants relies upon traditional plant breeding methods. Another approach involves genetic manipulation of plant characteristics through the introduction of exogenous nucleic acids conferring a desirable trait. Thus manipulation of the genes involved in amino acid synthesis and storage is useful for producing plants with increased or optimized amino acid content.

### Altered Carotene Content

Carotenoids are pigments with a variety of applications. They are yellow-orange-red lipids which are present in green plants, some molds, yeast and bacteria. Carotenoid hydrocarbons are referred to as carotenes, whereas oxygenated derivatives are referred to as xanthophylls. The carotenoids are part of the larger isoprenoid biosynthesis pathway which, in addition to carotenoids, produces such compounds as chlorophyll and tocopherols, which are Vitamin E active agents. The carotenoid pathway in plants produces carotenes, such as α- and β-carotene, and lycopene, and xanthophylls, such as lutein.

The biosynthesis of carotenoids involves the condensation of two molecules of the C₂₀ precursor geranyl PPᵢ to yield the first C₄₀ hydrocarbon phytoene. In a series of sequential desaturations, phytoene yields lycopene. Lycopene is the precursor of the cyclic carotenes, β-carotene and α-carotene. The xanthophylls, zeaxanthin and lutein are formed by hydroxylation of .beta.-carotene and .alpha.-carotene, respectively.

β-carotene, a carotene whose color is in the spectrum ranging from yellow to orange, is present in a large amount in the roots of carrots and in green leaves of plants. β-carotene is useful as a coloring material and also as a precursor of vitamin A in mammals. Current methods for commercial production of β-carotene include isolation from carrots, chemical synthesis, and microbial production.

A number of crop plants and a single oilseed crop are known to have substantial levels of carotenoids, and consumption of such natural sources of carotenoids has been indicated as providing various beneficial health effects. Thus, manipulation of the genes involved in carotenoid biosynthesis and storage would allow production of plants with increased carotenoid content and/or composition, for example increases in lutein, lycopene and carotene.

### Altered Tocopherol Content

Tocopherols and tocotrienols (unsaturated tocopherol derivatives) are well known antioxidants, and play an important role in protecting cells from free radical damage, and in the prevention of many diseases, including cardiac disease, cancer, cataracts, retinopathy, Alzheimer's disease, and neurodegeneration, and have been shown to have beneficial effects on symptoms of arthritis, and in anti-aging. Vitamin E (α-tocopherol) is used in chicken feed for improving the shelf life, appearance, flavor, and oxidative stability of meat, and to transfer tocols from feed to eggs. Vitamin E has been shown to be essential for normal reproduction, improves overall performance, and enhances immunocompetence in livestock animals. Vitamin E supplement in animal feed also imparts oxidative stability to milk products.

The demand for natural tocopherols as supplements has been steadily growing at a rate of 10-20% for the past three years. At present, the demand exceeds the supply for natural tocopherols, which are known to be more biopotent than racemic mixtures of synthetically produced tocopherols. Naturally occurring tocopherols are all d-stereomers, whereas synthetic α-tocopherol is a mixture of eight d,1-α-tocopherol isomers, only one of which (12.5%) is identical to the natural d-α-tocopherol. Natural d-α-tocopherol has the highest vitamin E activity (1.49 IU/mg) when compared to other natural tocopherols or tocotrienols. The synthetic α-tocopherol has a vitamin E activity of 1.1 IU/mg. In 1995, the worldwide market for raw refined tocopherols was $1020 million; synthetic materials comprised 85-88% of the market, the remaining 12-15% being natural materials. The best sources of natural tocopherols and tocotrienols are vegetable oils and grain products. Currently, most of the natural Vitamin E is produced from γ-tocopherol derived from soy oil processing, which is subsequently converted to α-tocopherol by chemical modification (α-tocopherol exhibits the greatest biological activity). Thus, methods of enhancing the levels of tocopherols and tocotrienols in plants, especially levels of the more desirable compounds that can be used directly, without chemical modification, would be useful to the art as such molecules exhibit better functionality and biovailability. This can be accomplished by manipulation of the genes involved in tocopherols and tocotrienols biosynthesis and storage.

### Altered Terpenoid Content

Terpenoids or terpenes represent a family of natural products found in most organisms (bacteria, fungi, animal, plants). Terpenoids are made up of five carbon units called isoprene units. They can be classified by the number of isoprene units present in their structure: monoterpenes (C10), sesquiterpenes (C15), diterpenes (C20), triterpenes (C30), tetraterpenes (C40) and polyterpenes (Cn). The plant kingdom contains the highest diversity of monoterpenes and sesquiterpenes, which are the most structurally diverse isoprenoids. They are usually volatile compounds and are mostly found in plants were they play a role in defense against pathogens and herbivores attacks, in pollinator attraction and in plant-plant communication.

Monoterpene and sesquiterpene accumulating plants have been of interest for thousands of years because of their flavor and fragrance properties and their cosmetic, medicinal and anti-microbial effects. The terpenes accumulated in the plants can be extracted by different means such as steam distillation that produces the so-called essential oil containing the concentrated terpenes. Such natural plant extracts are important components for the flavor and perfumery industry.

Some plants, known as aromatic plants or essential-oil-plants, accumulate large amounts of monoterpenes and sesquiterpenes in their leaves. In these plants, the terpenes are often synthesized and accumulated in specialized anatomical structures, glandular trichomes or secretory cavities, localized on the leaves and stems surface. Classical examples of such plants are members from the Lamiaceae family such as lavender, mint, sage, basil and patchouli.

The biosynthesis of terpenes in plants has been extensively studied. The common five-carbon precursor to all terpenes is isopentenyl pyrophosphate (IPP). Most of the enzymes catalyzing the steps leading to IPP have been cloned and characterized. Two distinct pathways for IPP biosynthesis coexist in the plants. The mevalonate pathway is found in the cytosol and endoplasmic reticulum and the non-mevalonate pathway (or deoxyxylulose (DXP) pathway) is found in the plastids. In the next step IPP is repetitively condensed by prenyl transferases to form the acyclic prenyl pyrophosphate terpene precursors for each class of terpenes, e.g. geranyl-pyrophosphate (GPP) for the monoterpenes, farnesyl-pyrophosphate (FPP) for the sesquiterpenes, geranylgeranyl-pyrophosphate (GGPP) for the diterpenes. These precursors serve as substrate for the terpene synthases or cyclases, which are specific for each class of terpene, e.g. monoterpene, sesquiterpene or diterpene synthases. Terpene synthases catalyze complex multiple step cyclizations to form the large diversity of carbon skeleton of the terpene compounds. Finally, in the last stage of terpenoid biosynthesis, the terpene molecules may undergo several steps of secondary enzymatic transformations such as hydroxylations, isomerisations, oxido-reductions or acylations, leading to the tens of thousand of different terpene molecules. Consequently, manipulation of the genes involved in terpene biosynthesis allows producing plants with modulated (i.e. increased or decreased) in terpene production and/or storage.

### Altered Carbohydrate Content

Sucrose is the carbon storage unit which is transported from the source tissues of most plants to the sink tissues. In sink tissues it is hydrolyzed and the components used to build other, more complex storage units, primarily starch, protein, and oil. The hydrolysis is primarily accomplished by sucrose synthase which produces UDPglucose and fructose. UDPglucose is converted to glucose 1-phosphate by UDPglucose pyrophosphorylase.

Altering the starch content of the sink tissues of various crop plants is desirable. Certainly, one such advantageous trait is enhanced starch and/or solids content and quality in various crop plants. For example, a more uniform distribution of starch and solids within the potato tuber or higher solids in the form of soluble (usually sugars and acids) and insoluble solids in tomatoes contribute to processing efficiency. Another advantageous trait is enhanced oil and protein content of seeds of various crop plants. Yet in some cases it is desirable to decrease the sucrose content of seeds in oilseed crops resulting in a decrease in the level of undesirable carbohydrates such as stachyose and raffinose, while increasing the carbon available for oil and protein production. Thus, manipulating the genes involved in starch and sugar biosynthesis allows producing plants with modulated (i.e. increased or decreased) in starch/sugar production and/or storage.

### Altered Lignin Content

Lignin is the major structural component of secondarily thickened plant cell walls. It is a complex polymer of hydroxylated and methoxylated phenylpropane units, linked via oxidative coupling that is probably catalyzed by both peroxidases and laccases (Boudet, et al., 1995. “Tansley review No. 80: Biochemistry and molecular biology of lignification,” New Phytologist 129:203-236). Lignin imparts mechanical strength to stems and trunks, and hydrophobicity to water-conducting vascular elements. Although the basic enzymology of lignin biosynthesis is reasonably well understood, the regulatory steps in lignin biosynthesis and deposition remain to be defined (Davin, L. B. and Lewis, N. G. 1992. “Phenylpropanoid metabolism: biosynthesis of monolignols, lignans and neolignans, lignins and suberins,” Rec Adv Phytochem 26:325-375; incorporated herein in its entirety).

There is considerable interest in the potential for genetic manipulation of lignin levels and/or composition to help improve digestibility of forages and pulping properties of trees. Small decreases in lignin content have been reported to positively impact the digestibility of forages. By improving the digestibility of forages, higher profitability can be achieved in cattle and related industries. In forestry, chemical treatments necessary for the removal of lignin from trees are costly and potentially polluting. Consequently, manipulating the genes involved in lignin biosynthesis allows producing plants with modulated (i.e. increased or decreased) lignin production and/or storage.

### Altered Cell Wall Content

The regulation of plant cell wall biosynthesis has various important significances in the fields of industry and agriculture. For example, by elevating cellulose/hemicellulose contents, the modification of plant cell wall components can lead to the production of plants that can supply fiber raw materials such as pulp and the improvement of the digestion/absorption efficiency of useful farm and feed crops. Thus, such modifications are significant from the aspects economical efficiency and profitability. Furthermore, structural modification of polysaccharides, which are cell wall components, can lead to the production of raw material plants having novel industrial values.

Mechanisms of cell wall synthesis have not been extensively and actively analyzed on the molecular level in spite of their industrial importance. Recently, molecular-level studies on plant cell wall synthesis are starting to be conducted using analytical techniques of molecular genetics. For example, it is though that the cell wall forms from Golgi body-derived vesicles which first fuse together near the cell division surface to form a phragmoplast. Callose then accumulates at that site forming a cell plate. Several proteins present in phragmoplasts and cell plates have been reported (M. Heese et al., Current Opinion in Plant Biology 1: 486-491 (1998); X. Gu and D. P. S. Verma, EMBO J. 15: 695-704 (1996)). Phragmoplastin is one of these proteins, termed the “dynamin-like protein” in general, and known to be broadly present in the whole plant and animal world (X. Gu and D. P. S. Verma, Plant Cell 9: 157-169 (1997); D. Otsuga, et al., J. Cell Biol. 143, 333-349 (1998); S. G. Kang et al., Plant Mol. Biol. 38: 437-447 (1998); D. C. Wienke et al., Molecular Biology of the Cell 10: 225-243 (1999)). However, the function of this protein has not been elucidated yet.

Other proteins reported as involved in cell wall synthesis include cellulose synthase and such (T. Arioli et al., “Molecular analysis of cellulose biosynthesis in Arabidopsis”, Science 279: 717-720 (1998)). However, many genes involved in cell wall synthesis are just now being isolated, and there are still many unknown mechanisms relating to cell wall synthesis (references: Y. Kawagoe and D. P. Delmer, “Pathways and genes involved in cellulose biosynthesis”, Genetic engineering 19, Plenum Press, New York (1997); K. Nishitani, “Construction and Restructuring of the cellulose-xyloglucan framework in the apoplast as mediated by the xyloglucan-related protein family-A hypothetical scheme”, J. Plant Res. 111: 159-166 (1998)). Consequently, manipulating the genes involved in cell wall biosynthesis allows producing plants with modulated cell wall structure and cell wall protein content.

### Altered Senescence Time

Senescence is the terminal phase of biological development in the life of a plant. It presages death and occurs at various levels of biological organization including the whole plant, organs, flowers and fruit, tissues and individual cells.

Cell membrane deterioration is an early and characteristic feature of senescence engendering increased permeability, loss of ionic gradients and decreased function of key membrane proteins such as ion pumps (Brown, et al., Plant Physiol.: A Treatise, Vol. X. Academic Press, 1991, pp. 227-275). Much of this decline in membrane structural and functional integrity can be attributed to lipase-mediated phospholipid metabolism. Loss of lipid phosphate has been demonstrated for senescing flower petals, leaves, cotyledons and ripening fruit (Thompson, J. E., Senescence and Aging in Plants, Academic Press, San Diego, 1988, pp. 51-83), and this appears to give rise to major alterations in the molecular organization of the membrane bilayer with advancing senescence that lead to impairment of cell function. There is growing evidence that much of the metabolism of lipids in senescing tissue is achieved through senescence-specific changes in gene expression (Buchanan-Wollaston, V., J. Exp. Bot., 1997, 307:181-199).

The onset of senescence can be induced by different factors both internal and external. For example, ethylene has been implicated as an internal regulator of leaf senescence in many plants. But evidence obtained from transgenic plants and ethylene response mutants indicates that although ethylene has an effect on senescence, it is not an essential regulator of the process.

External factors that induce premature initiation of senescence include environmental stresses such as temperature, drought, poor light or nutrient supply, as well as pathogen attack. As in the case of natural (age-related) senescence, environmental stress-induced senescence is characterized by a loss of cellular membrane integrity. Specifically, exposure to environmental stress induces electrolyte leakage reflecting membrane damage (Sharom, et al., 1994, Plant Physiol., 105:305-308; Wright and Simon, 1973, J. Exp. Botany, 24:400-411; Wright, M., 1974, Planta, 120:63-69; and Eze et al., 1986, Physiologia Plantarum, 68:323-328), a decline in membrane phospholipid levels (Wright, M., 1974, Planta, 120:63-69) and lipid phase transitions (Sharom, et al., 1994, Plant Physiol., 105:305-308), all of which can be attributed to the action of lipase.

Presently, there is no widely applicable method for controlling onset of senescence caused by either internal or external factors. At present, the technology for controlling senescence and increasing the shelf-life of fresh, perishable plant produce, such as fruits, flowers and vegetables relies primarily upon reducing ethylene biosynthesis. Consequently, manipulating the genes involved in senescence allows production of plants having improved shelf life.

### Altered UV Sensitivity

Flavonoids constitute a relatively diverse family of aromatic molecules that are derived from phenylalanine and malonyl-coenzyme A (CoA, via the fatty acid pathway). These compounds include six major subgroups that are found in most higher plants: the chalcones, flavones, flavonols, flavandiols, anthocyanins and condensed tannins (or proanthocyanidins). A seventh group, the aurones, is widespread, but not ubiquitous.

Some plant species also synthesize specialized forms of flavonoids, such as the isoflavonoids that are found in legumes and a small number of non-legume plants. Similarly, sorghum, maize and gloxinia are among the few species known to synthesize 3-deoxyanthocyanins (or phlobaphenes in the polymerised form). The stilbenes which are closely related to flavonoids, are synthesised by another group of unrelated species that includes grape, peanut and pine.

The major branch pathways of flavonoid biosynthesis start with general phenylpropanoid metabolism and lead to the nine major subgroups: the colorless chalcones, aurones, isoflavonoids, flavones, flavonols, flavandiols, anthocyanins, condensed tannins, and phlobaphene pigments. The enzyme phenylalanine ammonia-lyase (PAL) of the general phenylpropanoid pathway will lead to the production of cinnamic acid. Cinnamate-4-hydroxylase (C4H) will produce p-coumaric acid which will be converted through the action of 4-coumaroyl:CoA-ligase (4CL) to the production of 4-coumaroyl-CoA and malonyl-CoA. The first committed step in flavonoid biosynthesis is catalyzed by chalcone synthase (CHS), which uses malonyl CoA and 4-coumaryl CoA as substrates. Chalcone reductase (CHR) balances the production of 5-hydroxy- or 5-deoxyflavonoids. The next enzyme, chalcone isomerase (CHI) catalyses ring closure to form a flavanone, but the reaction can also occur spontaneously. Other enzymes in the pathway are: flavanone 3-hydroxylase (F3H), dihydroflavonol 4-reductase (DFR), flavonoid 3′-hydroxylase (F3′H) and flavonoid 3′,5′ hydroxylase (F3′5′H).

Besides providing pigmentation to flowers, fruits, seeds, and leaves, flavonoids also have key roles in signaling between plants and microbes, in male fertility of some species, in defense as antimicrobial agents and feeding deterrents, and in UV protection. While nucleic acid sequences encoding some flavonoid biosynthetic enzymes have been isolated for certain species of plants, there remains a need for materials useful in modifying flavonoid biosynthesis and UV-light absorption. Consequently, manipulating the genes involved in modifying flavonoid biosynthesis and UV-light absorption allows production of plants with improved responses to UV-light.

### Altered Protein Content

Proteins are important nutrients necessary for the building, maintenance and repair of animal tissues. Nine of the twenty amino acids constituting proteins cannot be produced by most animals and must be obtained from the diet. A variety of grains, seeds, legumes and vegetables can provide all the essential amino acids needed. Plant leaf proteins can supply large quantities of protein for animal feeds, and can offer a useful potential source of protein for human consumption.

Proteins are continuously synthesized and degraded in all living organisms, with half-lives ranging from as short as a few minutes to, weeks or more. The concentration of any individual protein is determined by the balance between its rates of synthesis and degradation, which in turn are controlled by a series of tightly-regulated biochemical mechanisms. Proteolysis in plant cells serves a variety of roles, including the control of cell cycle, the recycling of amino acids (e.g. seed germination), the degradation of polypeptides not folded properly, the elimination of foreign proteins, and many other cellular processes (Callis 1995; Estelle 2001). While proteolytic enzymes play vital roles in vivo, however, proteolysis by plant proteases is a severe problem affecting the nutritional quality of crops, notably during the ensiling process (McDonald 1981), or during the preparation of leaf protein concentrates (Jones et al. 1995).

At present, the most common strategy to avoid unwanted proteolysis in planta is to accumulate recombinant polypeptides in alternative cellular locations using appropriate targeting signals (Michaud et al. 1998). Alternatively, protein stability can be engineered by removing short amino acid domains involved in the control of protein turnover.

Another genetic engineering approach is to introduce into a DNA sequence encoding the small subunit of a ADP-glucose pyrophosphorylase (AGP). The expression of the AGP-encoding cDNA sequence in the antisense orientation, under the control of the seed-specific legumin B4 promoter, was shown to result in increased content of total nitrogen and protein (Weber et al. 2000). Consequently, manipulating the genes involved in protein biosynthesis and stability allows production of plants with improved protein content.

### Altered Nutritional Value

Grain is widely used as animal feed and human food and is a source of protein, starch and oil for many lower animals including swine, beef and dairy cattle, fish and poultry. In some countries, such as Mexico, over 70% of the harvested corn is used for human consumption, and corn and corn-derived products such as hominy and tortillas are dietary staples. In grain such as corn, as an example, the bulk of the amino acid composition is determined by the amount and type of amino acids contained in polypeptides. Only a relatively small portion, up to 10%, of the available amino acids in the kernel exists as free amino acid pools; the rest are contained in various proteins such as seed storage proteins. These seed storage proteins are synthesized as the grain kernal develops and are used as a source of energy as the kernel germinates and begins to grow. A similar situation exists in other crops.

Seed storage proteins, however, contain little to no lysine. Of the ten amino acids deemed essential in a mixed grain feed (arginine, histidine, isoleucine, leucine, lysine, methionine or cysteine, phenylalanine or tyrosine, threonine, tryptophan, and valine), corn is particularly lacking not only in lysine, but also in threonine and methionine. The lack of these essential amino acids, especially lysine, requires that feed corn be supplemented with these nutrients, often provided by the addition of soybean meal or synthetic lysine.

In human nutrition, the poor tend to consume diets that are relatively high in inexpensive starchy foods and relatively low in high quality protein. Kwashiorkor is a form of malnutrition caused by inadequate quality protein intake in the presence of fair to good energy (total calories) intake. Early symptoms are very general and include fatigue, irritability, and lethargy. As protein deficiency continues, growth failure, loss of muscle mass, generalized swelling (edema), and decreased immunity occur. A large, protuberant belly is common. Skin conditions (such as dermatitis, changes in pigmentation, thinning of hair, and vitiligo) are seen frequently. Shock and coma precede death. One government estimate suggests that as many as 50% of elderly persons in nursing homes in the U.S. suffer from protein-calorie malnutrition. Thus, grain products with improved protein quality due to higher levels of lysine, for example, would significantly improve not only the nutritional quality of the diet but the overall general level of health. Thus, manipulating the genes involved in amino acid biosynthesis and stability allows production of plants with improved nutritional value.

### Improved Abiotic Stress

Plants are constantly exposed to a variety of biotic (i.e., pathogen infection and insect herbivory) and abiotic (i.e., high or low temperature, drought, and salinity) stresses. To survive these challenges, plants have developed elaborate mechanisms to perceive external signals and to manifest adaptive responses with proper physiological and morphological changes (Bohnert et al., 1995). It would, therefore, be of great interest and importance to be able to manipulate the genes that confer abiotic stress tolerance to thereby create transformed plants (such as crop plants) with improved characteristics.

### Altered Morphology And Architecture

Plant architecture plays a very important role in overall crop performance. The characteristics of the inflorescence, flower, silique/fruit, and stem internodes have broad agronomic implications in the overall productivity of any crop plant. Compact architecture can contribute to productivity. For example, flowering stalks or inflorescences that are compact in nature and do not shade lower photosynthetic tissue can allow for greater productivity while a compact vegetative structure can allow a higher number of plants per square foot. Similarly, a vegetative structure, flowering stalk or inflorescence that is spread out may allow for more photosynthesis to take place during growth and/or seed development. Thus, different architectures may be desired for different crops.

Since most crop varieties have been derived directly or indirectly through breeding from wild species, productivity of crops may be affected by characteristics that are evolutionarily beneficial to wild species but impair performance in an agricultural setting. For example, well spread-out flowers and siliques with long pedicels on the inflorescence (along with genes controlling seed dispersal mechanisms such as shattering) may be evolutionarily beneficial to wild species, while in a crop setting this confers significant disadvantages in terms of overall productivity as measured by harvested seed.

Plant architecture or morphology is a major determining factor in plant productivity under agricultural settings. Plant varieties that have well-defined morphology of a uniform nature and pattern are preferred since they are amenable to mechanical cultivation. In particular, plant species that produce seed are selected for the uniformity of the placement of seed forming structures (typically seed pods or cobs) to allow efficient mechanical harvesting of seed. Plant varieties are also selected on the basis of other seed forming characteristics, such as strong pods to ensure no seed is lost or dispersed prior to harvesting, or compact nature of the raceme of the plant that contains the seedpods. Not all plants have these ideal characteristics. Thus, there is a strong interest in modifying plant archeticture. For example, compact plants, with clustered seed pods can provide many benefits for mechanical production of the crop, as well as lead to increased productivity. Accordingly, control of plant form and plant architecture is a desirable goal for the industry and manipulation of the genes involved in plant archeticture allow production of improved plant characteristics.

### Modified Seed Yield

Cytokinins have been demonstrated to play a fundamental role in establishing seed size, decreasing tip kernel abortion and increasing seed set during unfavorable environmental conditions. The first naturally occurring cytokinin was identified as 6-(4-hydroxy-3-methylbut-trans-2-enylamino) purine, more commonly known today as zeatin. In the main all naturally occurring cytokinins appear to be purine derivatives with a branched 5-carbon N⁶ substitutent. (See: McGaw, B. A., In: Plant Hormones and their Role in Plant Growth and Development, ed. P. J. Davies, Martinus Nijhoff Publ., Boston, 1987, Chap B3, Pgs. 76-93, the contents of which are incorporated by reference for purposes of background.) While some 25 different naturally occurring cytokinins have been identified, those regarded as particularly active are N⁶ (Δ²-isopentenyl) adenosine (iP), zeatin (Z), diHZ, benzyladenine (BAP) and their 9-ribosyl (and in the case of Z and diHZ, their 0-glucosyl) derivatives. However, such activity is markedly reduced in the 7- and 9-glucosyl and 9-alanyl conjugates. These latter compounds may be reflective of deactivation or control mechanisms.

The metabolism of cytokinins in plants is complex. Multi-step biochemical pathways are known for the biosynthesis and degradation of cytokinins. At least two major routes of cytokinin biosynthesis are recognized. The first involves transfer RNA (tRNA) as an intermediate. The second involves de novo (direct) biosynthesis. In the first case, tRNAs are known to contain a variety of hypermodified bases (among them are certain cytokinins). These modifications are known to occur at the tRNA polymer level as a post-transcriptional modification. The branched 5-carbon N⁶ substituent is derived from mevalonic acid pyrophosphate, which undergoes decarboxylation, dehydration, and isomerization to yield A²-isopentenyl pyrophosphate (iPP). The latter condenses with the relevant adenosine residue in the tRNA. Further modifications are then possible. Ultimately the tRNAs are hydrolyzed to their component bases, thereby forming a pool of available free cytokinins.

Alternately, enzymes have been discovered that catalyze the formation of cytokinins de novo, i.e., without a tRNA intermediate. The ipt gene utilized in the practice of this invention is one such gene. The formation of free cytokinins is presumed to begin with [9R5′P] iP. This compound is rapidly and stereospecifically hydroxylated to give the zeatin derivatives from which any number of further metabolic events may ensue. Such events include but are not limited to (1) conjugation, incorporating ribosides, ribotides, glucosides, and amino acids; (2) hydrolysis; (3) reduction; and (4) oxidation. While each enzyme in these pathways is a candidate as an effector of cytokinin levels, enzymes associated with rate-limiting steps have particular utility. Thus, manipulating the genes involved in cytokinin production and signaling allows production of plants with altered (i.e. increased or decreased) seed yield.

### Modified Seed Content

Plant seeds contain a number of different tissues including the embryo and cotyledons that are usually encased in a layer of thickened and lignified tissue referred to as a seed coat. In general, seed coats contain a significant portion of the total undigestible fiber content of plant seeds.

The seed coat provides a mechanical barrier that protects the seed prior to germination and allows the seed to remain dormant or withstand mechanical challenges. Some plant species have extremely strong seed coats that can withstand significant mechanical and environmental insult. Other plant species have thinner seed coats that offer a limited degree of protection from mechanical damage. The nature of the seed coat is determined genetically and is typically correlated with the biology and ecology of the plant species.

In many crop species of commercial interest, a thick seed coat is generally undesirable since the seed coat tends to contain a high level of undigestible fiber and is often a waste product upon processing of the seed for oil, meal or other products. The seed coat contributes a significant portion of the fiber content to plant seed meals. Thus, reduction of the seed coat is an important goal for crop improvement in many crop species. However, the importance of the seed coat for the protection of the seed itself dictates that any reduction in seed coat still allow for the protection of the seed from injury or damage during seed harvesting, processing, or planting of seed. Plant seeds with reduced fiber content provide many advantages for use as feed products. Thus, alteration of the seed coat composition, as well as alteration of the composition of the other tissues of the seed for reduced fiber content, can provide an improvement for plant seeds currently used for feed.

### Enhanced Heat Tolerance

Exposure to heat stress often causes the perturbation of diverse biological processes and thus results in reduced plant yield and overall decreased quality. (Maestri et al. (2002) Plant Molecular Biology 48: 667-681). Under heat stress, plants succumb to a variety of physiological and developmental damages, including dehydration due to high transpiration, impairment of photosynthetic carbon assimilation, inhibition of translocation of assimilates, increased respiration, decrease in the duration of developmental phases leading to smaller organs, disruption of seed development and reduction of fertility (Berry and Björkman (1980) Annu Rev Plant Physiol 31: 491-543; Cheikh and Jones (1994) Plant Physiol 106: 45-51). Heat stress can cause profound and complex cellular effects in plants, such as increasing membrane fluidity and permeability, protein aggregation and denaturing enzymes. These cellular damages eventually lead to defects of plant development and growth and even death under high temperature. Although it is unclear how plants sense heat, an increasing amount of evidence has indicated that thermotolerance, including basal thermotolerance and acquired thermotolerance, involves multiple signaling pathways and cellular components (Larkindale et al. (2005) Plant Physiol 138: 882-897). A crosstalk has been reported between heat shock stress and dehydration/drought, cold/chilling/freezing, heavy metal stress, hormonal regulation and oxidative stress in plants.

The best-known mechanism in plants and other organisms to cope with heat stress is the rapid synthesis of heat shock proteins (HSPs). In plants, there are a large number of heat shock proteins that can be classified into multiple protein families based on molecular mass. Heat shock proteins have been implicated in serving as molecular chaperons to protect organelles and enzymes and renature proteins under high temperature to restore cellular homeostasis. The heat shock response is primarily regulated at the transcriptional level. The expression of some heat shock genes are rapidly induced in heat response that is mediated by heat transcription factors (HSFs), while others present in various tissues and organs in plants under normal non-heat stress conditions, indicating these proteins perform other fundamental roles in plant growth and development as well. HSFs bind to conserved cis-regulatory promoter elements (HSEs), which result in an increase of heat shock protein synthesis. (Wang et al. (2003) Planta 218:1-14). Although overexpression of HSFs can induce constitutive expression of downstream heat stress-associated HSPs, HSFs may also activate non-heat stress genes that adversely affect the normal agronomic characteristics of a plant. Thus, manipulation of the genes involved in the thermotolerance pathway allows for production of plants with improved growth and development under heat stress conditions.

### Improved Oxidative Stress

Stress conditions, such as extremes in temperature, drought and desiccation, salinity, soil nutrient content, heavy metals, UV radiation, pollutants such as ozone and SO₂, mechanical stress, high light and pathogen attack, induce generation of toxic oxygen species which are first found in the stressed cells and which then spread to the whole organism. Toxic oxygen species are partially reduced or activated derivatives of oxygen (e.g. reactive oxygen species (ROS), reactive oxygen intermediates (ROI) or activated oxygen species (AOS)) At high levels these cause severe damage to the cell membrane, protein and DNA, and may result in cell death. Several recently published reports have characterized toxic oxygen species generation and the subsequent oxidative damage caused by abiotic stresses (see Larkindale and Knight (2002); Borsani et al. (2001); Lee et al (2004); Aroca et al (2005); Luna et al (2005); and Noctor et al (2002)).

Although capable of producing damage, ROS/ROI/AOS are also key regulators of metabolic and defense pathways, playing roles as signaling or secondary messenger molecules. In the signal cascades leading to oxidative stress, salicylic acid (SA) has been identified as an important signaling molecule to mediate ROS/ROI/AOS accumulation in various stress conditions, such as salt and osmotic stress (Borsani et al. (2001)), drought (Senaratna et al. (2000)), heat (Dat et al. (1998)), cold (Scott et al. (2004)), UV-light (Surplus et al. (1998)), paraquat (Kim et al. (2003)) and disease resistance against different pathogens (Zhou et al. (2004)). High levels of SA induce H₂O₂ production as well as cell death.

Similarly, NO is capable of generating ROS/ROI/AOS and is a plant signaling molecule involved in the regulation of seed germination, stomatal closure (Mata and Lamattina (2001); Desikan et al (2002)), flowering time (He et al. (2004)), antioxidant reactions to suppress cell death (Beligni et al. (2002)) and tolerance to biotic and abiotic stress conditions (Mata and Lamattina (2001)). While the effects of NO can be mimicked through the application of sodium nitroprusside (SNP), endogenous NO production in plants results from the activity of a nitric oxide synthase that uses L-arginine (Guo et al. (2003)) as well as nitrate reductase-mediated reactions (Desikan et al (2002)). NO can react with redox centers in proteins and membranes, thereby causing cell damage and inducing cell death.

In order to control the two-fold nature of ROS/ROI/AOS molecules, plants have developed a sophisticated regulatory system which involves both production and scavenging of ROS/ROI/AOS in cells. Plant development and yield depend on the ability of the plant to manage oxidative stress, whether it is via the signaling or the scavenging pathways. Consequently, improvements in a plant's ability to withstand oxidative stress, or to obtain a higher degree of cross-tolerance once oxidative stress has been experienced, has significant value in agriculture.

### Altered Flowering Times

The life cycle of flowering plants in general can be divided into three growth phases: vegetative, reproductive and seed development. If the appropriate environmental and developmental signals that induce the plant to switch to floral or reproductive growth are disrupted, the plant will not be able to enter reproductive growth and will maintain vegetative growth.

Temporal coordination of life cycle stages depends on factors such as energy requirements, environmental variables and reproductive strategy. Maximization of any one life cycle period or stage is usually at the cost of one of the other periods or stages in the life cycle. For instance, trade-offs exist between the initiation of the reproductive growth stage and the length of the vegetative growth stage, the early flowering and the later growth and reproduction stages. Thus, early flowering in plants can provide discernable advantages over other later flowering plants. For instance, early flowering usually results in early maturity and eventually shortens growth duration from sowing to harvest in plants. This allows farmers to avoid environmental adversity, such as freezing temperatures, in the early or later growing seasons at high latitude or altitude. This competitive advantage can also help crop rotation schedules. Alternatively, if cold weather or crop rotation is not a problem, later flowering has other advantages, such as a robust vegetative state, yielding higher amounts of plant material.

These advantages to the plant also translate into economic and production advantages providing more efficient human use of plants and especially plant crops critical to human survival. Generally, early flowering plants have smaller plant stature due to shortened vegetative growth compared to wild-type plants. Small plant stature is desirable in some cases. However, technologies enabling alteration of the moment within the life cycle at which a plant flowers are more advantageous if this alteration is accomplished without other disadvantageous trade-offs, such as a reduction in plant mass or other changes in phenotypically discernable traits. Consequently, manipulating the genes involved in flower development allows production of altered (i.e. earlier or later) flowering time.

### Improved Nitrogen Use Efficiency

Nitrogen is most frequently the rate limiting mineral nutrient for crop production and all field crops have a fundamental dependence on exogenous nitrogen sources. Nitrogenous fertilizer, which is usually supplied as ammonium nitrate, potassium nitrate or urea, typically accounts for 40% of the costs associated with crops in intensive agriculture, such as corn and wheat. Increased efficiency of nitrogen use by plants enables the production of higher yields with existing fertilizer inputs, enables existing crop yields to be obtained with lower fertilizer input or enables better yields from soils of poorer quality (Good et al. (2004) Trends Plant Sci. 9:57-605). Higher amounts of proteins in the crops can also be produced more cost-effectively.

Plants have a number of means to cope with nitrogen nutrient deficiencies, such as poor nitrogen availability. One important mechanism senses nitrogen availability in the soil and responds accordingly by modulating gene expression while a second mechanism is to sequester or store nitrogen in times of abundance to be used later. The nitrogen sensing mechanism relies on regulated gene expression and enables rapid physiological and metabolic responses to changes in the supply of inorganic nitrogen in the soil by adjusting nitrogen uptake, reduction, partitioning, remobilization and transport in response to changing environmental conditions. Nitrate acts as a signal to initiate a number of responses that serve to reprogram plant metabolism, physiology and development (Redinbaugh et al. (1991) Physiol. Plant. 82, 640-650.; Forde (2002) Annual Review of Plant Biology 53, 203-224). Nitrogen-inducible gene expression has been characterized for a number of genes in some detail. These include nitrate reductase, nitrite reductase, 6-phosphoglucante dehydrogenase, and nitrate and ammonium transporters (Redinbaugh et al. (1991) Physiol. Plant. 82, 640-650; Huber et al. (1994) Plant Physiol 106, 1667-1674; Hwang et al. (1997) Plant Physiol. 113, 853-862; Redinbaugh et al. (1998) Plant Science 134, 129-140; Gazzarrini et al. (1999) Plant Cell 11, 937-948; Glass et al. (2002) J. Exp. Bot. 53, 855-864; Okamoto et al. (2003) Plant Cell Physiol. 44, 304-317).

Inefficiencies in nitrogen use efficiency (NUE) may be overcome through the use of nitrogen regulated gene expression to modify the response of rate limiting enzymes and metabolic pathways that occur in response to changes in nitrogen availability. General reviews of these pathways and processes can be found in: Derlot et al. (2001) Amino Acid Transport. In Plant Nitrogen (eds. Lea and Morot-Gaudry), pp. 167-212. Springer-Verlag, Berlin, Heidelberg; Glass et al. (2002) J. Exp. Bot. 53: 855-864; Krapp et al. (2002) Nitrogen and Signaling. In Photosynthetic Nitrogen Assimilation and Associated Carbon Respiratory Metabolism (eds. Foyer and Noctor), pp. 205-225. Kluwer Academic Publisher, Dordrecht, The Netherlands; and Touraine et al. (2001) Nitrate uptake and its regulation. In Plant Nitrogen (eds. Lea and Morot-Gaudry), pp. 1-36. Springer-Verlag, Berlin, Heidelberg. Overcoming the rate limiting steps in nitrogen assimilation, transport and metabolism has the effect of increasing the yield, reducing the nitrogen content and reducing the protein content of plants grown under nitrogen limiting conditions.

### Modulated Chilling (Cold) Sensitivity

Plants exposed to cold or chilling conditions typically have low yields of biomass, seeds, fruit and other edible products. The term “chilling sensitivity” is used for the description of physiological and developmental damages in the plant caused by low, but above freezing, temperatures. In some countries or agricultural regions of the world chilling temperatures are a significant cause of crop losses and a primary factor limiting the geographical range and growing season of many crop species. In addition, poor germination and reduced growth of chilling sensitive crops in the spring results in less ground coverage, more erosion and increased occurrence of weeds leading to less nutrient supply for the crop.

Typically, chilling damage includes wilting, necrosis or ion leakage from cell membranes, especially calcium leakage, and decreased membrane fluidity, which consequently impacts membrane dependent processes such as: photosynthesis, protein synthesis, ATPase activity, uptake of nitrogen, etc. (see Levitt J (1980) Chilling injury and resistance. In Chilling, Freezing, and High Temperature Stresses: Responses of Plant to Environmental Stresses, Vol 1., T T Kozlowsky, ed, Academic Press, New York, pp 23-64; Graham and Patterson (1982) Annu Rev Plant Physiol 33: 347-372; Guy (1990) Annu Rev Plant Physiol Plant Mol Biol 41: 187-223; and Nishida and Murata (1996) Annu Rev Plant Physiol Plant Mol Biol 47: 541-568.). In addition, cold temperatures are often associated with wet conditions. The combination of cold and wet can result in hypoxic stress on the roots, causing an even more severe reduction of growth rate but, more critically, can be lethal to the plants, especially sensitive plant species such as corn and cotton.

Yet it has been observed that environmental factors, such as low temperature, can serve as triggers to induce cold acclimation processes allowing plants responding thereto to survive and thrive in low temperature environments. It would, therefore, be of great interest and importance to produce plants with improved cold tolerance characteristics such as faster germination and/or growth and/or improved nitrogen uptake under cold conditions to improve survival or performance under low or chilling temperatures.

### Modulated Drought Sensitivity

Plants cannot grow without sufficient water. While nutrient availability plays a critical role in plant growth and development, these nutrients must be in aqueous form. In addition, many marginal growing regions may have an adequate nutrient supply, but without enough water to allow maintenance of plant turgor and membrane integrity, such lands cannot be maximally cultivated. Increased plant drought tolerance enables the production of higher yields from such lands and/or enables existing yields of crops to be obtained with lower water input. As a consequence, crops are produced more cost-effectively.

One of the major consequences of drought is the loss of water from the protoplasm, which leads to increased ion concentrations within the cell. At high concentrations ions such as chlorine and nitrate inhibit metabolic functions (Hartung et al (1998) Prog Bot 59:299-327). Eventually a “glassy state” results from cell water loss and the concentration of protoplasmic constituents. Here, the remaining cell liquid is highly viscous, which increases the chances of protein denaturation and membrane fusion due to abnormal molecular interactions (see: Hartung et al. (1998) Prog Bot 59:299-327 and Hoekstra et al. (2001) Trends Plant Sci 6:431-438). This indicates that the ability to maintain cell turgor and metabolism is genetically encoded.

### Moduated Phosphate Use Efficiency

Severe loss in economically valuable agricultural and ornamental plants is caused by phosphate starvation. As an essential nutrient, phosphate is required for many key processes in plants such as carbohydrate metabolism, photosynthesis, respiration and signal transduction. Phosphate is also required for the biosynthesis of essential molecules such as adenylates, RNA, DNA, membrane lipids and others. For optimal plant growth, development, production and output, phosphate must be efficiently mobilized from the soil, taken up by roots, transported, distributed and re-distributed in the plant. The effect of low-phosphate conditions, therefore, is significantly deleterious to many aspects of plant growth, development, production, biomass and/or output.

Severe loss in economically valuable agricultural and ornamental plants is also caused by alkaline soil. Soil pH has a significant effect on the solubility of essential plant minerals and nutrients. Fourteen of seventeen essential plant nutrients are found in soil, but these nutrients are only available to the plant when in soluble form. As soil alkalinity increases, many essential nutrients become more insoluble and unavailable to the plant, thereby limiting plant growth, development, production and output. By way of example, the availability of nitrogen, phosphorus, copper, boron, manganese and zinc are known to decrease at high pH levels.

Human activities and natural geological processes have created vast tracts of phosphate depleted and/or alkaline soils that would otherwise be agriculturally productive. For these and other reasons, plants with improved phosphate use efficiency would be economically important.

### Modulated Salt Sensitivity

A wide variety agriculturally important plant species demonstrate significant sensitivity to saline water and/or soil. Upon salt concentration exceeding a relatively low threshold, many plants suffer from stunted growth, necrosis and/or death that results in an overall stunted appearance and reduced yields of plant material, seeds, fruit and other valuable products. Physiologically, plants challenged with salinity experience disruption in ion and water homeostasis, inhibition of metabolism and damage to cellular membranes that result in developmental arrest and cell death (Huh et al. (2002) Plant J, 29(5):649-59).

In many of the world's most productive agricultural regions, agricultural activities themselves lead to increased water and soil salinity, which threatens their sustained productivity. One example is crop irrigation in arid regions that have abundant sunlight. After irrigation water is applied to cropland, it is removed by the processes of evaporation and evapotranspiration. While these processes remove water from the soil, they leave behind dissolved salts carried in irrigation water. Consequently, soil and groundwater salt concentrations build over time, rendering the land and shallow groundwater saline and thus damaging to crops.

In addition to human activities, natural geological processes have created vast tracts of saline land that would be highly productive if not saline. In total, approximately 20% of the irrigated lands in the world are negatively affected by salinity. (Yamaguchi and Blumwald, 2005, Trends in Plant Science, 10: 615-620). For these and other reasons, plants (such as crop plants) with enhanced growth and/or productivity characteristics in saline conditions are desirable.

### Modulated Low Light and Light Quality Response

When plants are too close together the crowding elicits a number of developmental responses, such as stem and petiole elongation, branch suppression and accelerated floweing (Smith, H. 1982, Light quality, photoreception and plant strategy. Annu. Rev. Pl. Physiol. 33: 481-518 and Schmitt, J. and R., D., Wulff 1993, Light spectral quality, phytochrome and plant competition. Trends Ecol. Evol. 8:47-50). This shade avoidance response is triggered by the reduced ratio of red to far red wavelengths (R:FR) transmitted through or reflected from green vegetation due to selective absorption of visible wavelengths by chlorophyll (see Smith 1982, above).

It is the phytochrome family of photoreceptors that senses these environmental variaions in the R:FR ratio. Phytochromes reversibly switch between R and FR-absorbing forms and interacts with multiple signaling pathways, such as the auxin pathway, to provide a dynamic response to shade (see Smith 1982 and Smith, H. 1995, Physiological and ecological function within the phytochrome family. Annu. Rev. Plant Physiol. Plant Mol. Biol. 46:289-315).

Shade tolerance, or the ability to tolerate extended periods of low light, varies from species to species. Photosynthesis is decreased in shade. As a consequence, in species such as turfgrasses this results in decreased carbohydrate reserves and reduced root, rhizome and tiller growth. In the turfgrass industry this is problematic because about 20-25% of turfgrasses are grown under low light conditions and a considerable amount of time and money is spent by golf courses in an effort to maintain quality turf under shade conditions.

Shade intolerance (shade avoidance) is detrimental to crop plants because the growth and performance of crop plants depends largely on crop architecture, and plant architecture is affected by reduced light. That is, densely planted crops that shade one another tend to place energy into stem and petiole elongation to lift the leaves into the sunlight rather than putting energy into storage or reproductive structures. This negatively affects yields by reducing the amount of harevestable products such as seeds, fruits and tubers. In addition, tall spindly plants tend to be less wind resistant and fall over easily, further reducing crop yield.

Likewise, shade intolerance negatively affects forestry plantings. Here, seedlings of shade tolerate species will self-prune at a slower rate and survive for longer periods under a dense forest canopy than shade intolerant trees. Since most commercially important tree species are shade intolerant to only moderately tolerant of shade, tree plantings must be less dense and require increased acreage. Thus, manipulation of the genes involved in shade tolerance and/or shade avoidance allows production of plants with improved growth under varying light conditions.

### Modulated Endosperm Cell Size

The endosperm, a characteristic formation of Angiosperm seeds, is a nutritive tissue for the embryo. In maize, the endosperm originates with series of free-nuclear divisions, followed by cellularization and the subsequent formation of a range of functional cellular domains. This tissue is complex in its structure and development, in particular in cereals.

The endosperm is the main storage organ in maize seeds, for example, nourishing the embryo while the seed develops, and providing nutrients to the seedling upon germination. Thus, the uptake of assimilates by the growing endosperm is a critical process in seed development.

The central area of the endosperm consists of large cells with vacuoles, which store the reserves of starch and proteins, whilst the region surrounding the embryo is distinguished by rather small cells, occupied for the major part by cytoplasm. The Basal Endosperm Transfer Layer (BETL) area is highly specialized to facilitate uptake of solutes during grain development. These transfer cells of the basal endosperm have specialized internal structures adapted to absorb solutes from the maternal pedicel tissue, and translocate these products to the developing endosperm and embryo. Thus, manipulation of genes involved in the pathway leading to endosperm cell generation and growth would allow production of plants with modulated endosperm cell size.

### Modulated Photosynthesis

Growth and productivity of crop plants are the main parameters of concern to a commercial grower. Such parameters are affected by numerous factors including the nature of the specific plant and allocation of resources within it, availability of resources in the growth environment and interactions with other organisms including pathogens.

Growth and productivity of most crop plants are limited by the availability of CO₂ to the carboxylating enzyme ribulose 1,5-bisphosphate carboxylase/oxygenase (Rubisco). Such availability is determined by the ambient concentration of CO₂ and stomatal conductance, and the rate of CO₂ fixation by Rubisco as determined by the Km(CO₂) and Vmax of this enzyme.

In C3 plants, the concentration of CO₂ at the site of Rubisco is lower than the Km(CO₂) of the enzyme, particularly under water stress conditions. As such, these crop plants exhibit a substantial decrease in growth and productivity when exposed to low CO₂ conditions induced by, for example, stomatal closure which can be caused by water stress. Many photosynthetic microorganisms are capable of concentrating CO₂ at the site of Rubisco to thereby overcome the limitation imposed by the low affinity of Rubisco for CO₂.

Higher plants of the C4 and the crassulacean acid metabolism (CAM) physiological groups can also raise the concentration of CO₂ at the site of Rubisco by means of dual carboxylations which are spatially (in C4) or temporally (in CAM) separated.

Since plant growth and productivity especially in C3 crop plants are highly dependent on CO₂ availability to Rubisco and fixation rates, numerous attempts have been made to genetically modify plants in order to enhance CO₂ fixation therein in hopes that such modification would lead to an increase in growth or yield. As such, numerous studies attempted to introduce the CO₂ concentrating mechanisms. of photosynthetic bacteria or C4 plants into C3 plants, so far with little or no success.

Although theoretically such approaches can lead to enhanced CO₂ fixation in C3 plants, results obtained from such studies have been disappointing. Thus there is a widely recognized need for, and it would be highly advantageous to have, a method of generating plants and crops exhibiting enhanced photosynthesis, growth and/or increased commercial yields.

### Modulated Light Quality Response

Flowering plants are subject to photoperiodism which is generally defined as the response of plants and animals to relative lengths of day and night. Plants are also sensitively attuned to differences in light quality. Red light, Far red light and blue light receptors are well characterized across plant species. One aspect of plant physiology that is particularly affected by photoperiodism and light quality is flowering.

The transition to flowering in plants is regulated by environmental factors such as temperature and light. In Arabidopsis thaliana, much is known about the photoperiod pathway that induces flowering in response to an increase in daylength. In contrast, the mechanisms that regulate flowering in response to changes in light quality are largely unknown. In crowded or shaded environments, the red/far-red ratio of incoming light reaching plants decreases, and a series of responses known collectively as the “shade avoidance syndrome” are triggered, including the promotion of stem elongation and acceleration of flowering (Ballare, C. L. Trends Plant Sci 4, 201, 1999; and Halliday, K. J., et al. Plant Physiol 104, 1311-1315, 1994). Phytochromes are a family of red/far-red-light photoreceptors essential for the perception of changes in light quality and shade avoidance responses; among the 5 phytochromes in Arabidopsis, a phytochrome B (phyB) plays the most significant role in the shade avoidance syndrome. The mechanisms by which phyB regulates flowering are largely unknown.

The phytochromes and the blue/UV-A photoreceptors called cryptochromes (cry1 and cry2 in Arabidopsis) are the most critical photoreceptors that regulate floral induction (Lin, C. Plant Physiol 123, 39-50, 2000). Several components involved in phytochrome signaling in seedlings have been isolated and characterized in recent years (Quail, P. H. Nat Rev Mol Cell Biol 3, 85-93, 2002). Seedlings defective in phytochrome A (phyA) signaling are tall under far-red light (FR) while plants defective in phyB signaling are tall under red-light (R). Despite the large number of components identified, it remains unclear how they are assembled into a signaling network. ELF3 and GI have been reported to have a role in flowering (Liu, X. L., et al. Plant Cell 13, 1293-304, 2001), mainly through mis-regulation of the circadian clock (Suarez-Lopez, P., et al. Nature 410, 116-20, 2001), but the mechanisms by which phytochromes regulate flowering directly are largely unknown. Thus, manipulating the genes involved in the light quality pathway allows production of plants with altered responses to light quality and able to grow better under sub-optimal conditions.

### Modulated Biomass

Many plants are specifically improved for agriculture, horticulture, biomass conversion, and other industries (e.g. paper industry, plants as production factories for proteins or other compounds). Modulation of the size and stature of an entire plant, or a particular portion of a plant, allows production of plants better suited for a particular industry. For example, reductions in the height of specific crops and tree species can be beneficial by allowing easier harvesting. Alternatively, increasing height, thickness or organ number may be beneficial by providing more biomass useful for processing into food, feed, fuels and/or chemicals. Other examples of commercially desirable traits include increasing the length of the floral stems of cut flowers, increasing or altering leaf size and shape or enhancing the size of seeds and/or fruits. Changes in organ size, organ number and biomass also result in changes in the mass of constituent molecules such as secondary products and convert the plants into factories for these compounds. Thus, manipulating the genes involved in morphology and the growth and development pathways allows production of plants with increased biomass.

### Modulated Seed Size

Seeds are the reproduction unit of higher plants. Plant seeds contain reserve compounds to ensure nutrition of the embryo after germination. These storage organs contribute significantly to human nutrition as well as cattle feeding. Seeds consist of three major parts, namely the embryo, the endosperm and the seed coat. Reserve compounds are deposited in the storage organ which is either the endosperm (resulting form double fertilization; e.g. in all cereals), the so-called perisperm (derived from the nucellus tissue) or the cotyledons (e.g. bean varieties). Storage compounds are lipids (oil seed rape), proteins (e.g. in the aleuron of cereals) or carbohydrates (starch, oligosaccharides like raffinose).

Starch is the storage compound in the seeds of cereals. The most important species are maize (yearly production ca. 570 mio t; according to FAO 1995), rice (540 mio t p.a.) and wheat (530 mio t p.a.). Protein rich seeds are found in different kinds of beans (Phaseolus spec., Vicia faba, Vigna spec.; ca. 20 mio t p.a.), pea (Pisum sativum; 14 mio t p.a.) and soybean (Glycine max. 136 mio t p.a.). Soybean seeds are also an important source of lipids. Lipid rich seeds are as well those of different Brassica species (app. 30 mio t p.a.), cotton, oriental sesame, flax, poppy, castor bean, sunflower, peanut, coconut, oilpalm and some other plants of less economic importance.

After fertilization, the developing seed becomes a sink organ that attracts nutritional compounds from source organs of the plant and uses them to produce the reserve compounds in the storage organ. Increases in seed size and weight, are desirable for many different crop species. In addition to increased starch, protein and lipid reserves and hence enhanced nutrition upon ingestion, increases in seed size and/or weight and cotyledon size and/or weight are correlated with faster growth upon germination (early vigor) and enhanced stress tolerance. Cytokinins are an important factor in determining sink strength. The common concept predicts that cytokinins are a positive regulator of sink strength. Consequently, manipulating the genes involved in seed development, including seed storage proteins and lipid production, as well as genes involved in cytokinin production and signaling allow production of plants with altered seed size.

### Modulated Seedling Growth

Plant hormone abscisic acid (ABA) controls various aspects of plant growth and development. It inhibits germination and postgermination growth at high concentrations, although it is necessary for normal seedling growth. It regulates seed maturation process and prevents embryos from precocious germination. During vegetative growth, ABA plays an essential role in adaptation to various abiotic stresses such as drought, high salinity and cold. Extensive genetic and biochemical studies have been done to identify the regulatory components of various aspects of ABA response. As a consequence, a large number of ABA signaling components have been reported that include transcription factors, kinases/phosphatases, RNA-binding proteins, G-proteins, and secondary messengers (Finkelstein et al., 2002; Xiong et al., 2002, both of which are hereby incorporated by reference in their entirety).

During vegetative growth, ABA controls the expression of numerous genes associated with adaptive responses to drought and other abiotic stresses (Ramanulu and Bartels, 2002; Shinozaki et al., 2003). The ABA-regulation of stress-responsive genes is largely mediated by cis-regulatory elements sharing the CACGTGGC consensus. A small subfamily of basic leucine zipper (bZIP) class transcription factors that interact with the elements have been identified (Choi et al., 2000; Uno et al., 2000). The factors, named as ABFs (i.e., ABF1-ABF4) or AREBs (i.e., AREB1-AREB3), are involved in ABA and various abiotic stress responses (Kang et al., 2002; Kim et al., 2004). In particular, ABF2/AREB 1, regulates seedling growth rate and plays an essential role in glucose-induced developmental arrest process. Thus, manipulating the genes involved in ABA production and signaling allow production of plants with improved seedling growth.

### Modulating Abscission

Abscission refers to the process by which a plant intentionally drops one or more of its parts or organs. One model for abscission provides that auxin renders cells in the abscission zone insensitive to ethylene, thereby preventing abscission. But when auxin concentration drops below a threshold level (likely set by relative ethylene concentrations), cells in the abscission zone perceive ethylene as an abscission-inducing signal. Activation of the abscission pathway results in the expression and/or activation of cell wall degrading enzymes, such as pectinases and cellulases, which structurally weaken the plant part or organ to be dropped.

Abscission is an important process in agriculturally and ornamentally valuable plants. It follows that the modulation of abscission, whether for the purpose of inducing or inhibiting the dropping of a plant part or organ, has important and wide-ranging applications. For instance, the flowering shelf-life of ornamental plants may be extended by inhibiting abscission. Consequently, manipulating the genes involved in activating and progressing the abscission pathway would allow production of plants with improved characteristics.

### Lethal Plants for Genetic Confinement Systems

As more and more transgenic plants are developed and introduced into the environment, it can be important to control the undesired spread of the transgenic triat(s) from transgenic plants to other traditional and transgenic cultivars, plant species and breeding lines, thereby preventing cross-contamination. The use of a conditionally lethal gene, i.e. one which results in plant cell death under certain conditions, has been suggested as a means to selectively kill plant cells containing a recombinant DNA (see e.g., WO 94/03619 and US patent publication 20050044596A1). The use of genes to control transmission and expression of transgenic traits is also described in U.S. application Ser. No. 10/667,295, filed on Sep. 17, 2003, which is hereby incorporated by reference. Some of the nucleotides of the invention are lethal genes, and can therefore be used as conditionally lethal genes, namely genes to be expressed in response to specific conditions, or in specific plant cells. For example, a gene that encodes a lethal trait can be placed under that control of a tissue specific promoter, or under the control of a promoter that is induced in response to specific conditions, for example, a specific chemical trigger, or specific environmental conditions.

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

What is claimed is:

1. An isolated nucleic acid molecule comprising:
a) a nucleic acid having a nucleotide sequence which encodes an amino acid sequence exhibiting at least 85% sequence identity to an amino acid sequence in the Sequence Listing; or
b) a nucleic acid which is an interfering RNA to the nucleotide sequence according to subparagraph (a).

2. The isolated nucleic acid molecule according to claim 1, which has the nucleotide sequence according to any polynucleotide sequence in the Sequence Listing.

3. The isolated nucleic acid molecule according to claim 1, wherein said amino acid sequence comprises any polypeptide sequence in the Sequence Listing.

4. A vector construct comprising:
a) a first nucleic acid having a regulatory sequence capable of directing transcription and/or translation in a plant; and
b) a second nucleic acid having the sequence of the isolated nucleic acid molecule according to claim 1;
wherein said first and second nucleic acids are operably linked.

5. The vector construct according to claim 4, wherein said first nucleic acid is native to said second nucleic acid.

6. The vector construct according to claim 4, wherein said first nucleic acid is heterologous to said second nucleic acid.

7. A host cell comprising an isolated nucleic acid molecule according to claim 1 wherein said nucleic acid molecule is flanked by exogenous sequence.

8. A host cell comprising the vector construct according to claim 4.

9. An isolated polypeptide comprising an amino acid sequence exhibiting at least 85% sequence identity with an amino acid sequence of the Sequence Listing.

10. A method of introducing an isolated nucleic acid molecule into a host cell comprising:
a) providing an isolated nucleic acid molecule according to claim 1; and
b) contacting said isolated nucleic acid molecule with said host cell under conditions that permit insertion of said nucleic acid into said host cell.

11. A method of transforming a host cell which comprises contacting a host cell with the vector construct according to claim 4.

12. A method for detecting a nucleic acid in a sample which comprises:
a) providing an isolated nucleic acid molecule according to claim 1;
b) contacting said isolated nucleic acid molecule with a sample under conditions which permit a comparison of the sequence of said isolated nucleic acid molecule with the sequence of DNA in said sample; and
c) analyzing the result of said comparison.

13. A plant, plant cell, plant material or seed of a plant which comprises a nucleic acid molecule according to claim 1 which is exogenous or heterologous to said plant or plant cell.

14. A plant, plant cell, plant material or seed of a plant which comprises the vector construct according to claim 4.

15. A plant that has been regenerated from the plant cell or seed according to claim 13 or 14.

16. A plant cell comprising an exogenous nucleic acid, said exogenous nucleic acid comprising a regulatory region operably linked to a polynucleotide that is transcribed into an interfering RNA effective for inhibiting expression of a polypeptide selected from the group consisting of a polypeptide having 85% or greater sequence identity to an amino acid sequence in Sequence Listing; wherein a tissue of a plant produced from said plant cell has a difference in polypeptide content as compared to the corresponding polypeptide content in tissue of a control plant that does not comprise said nucleic acid.

17. A method of modulating a plant growth or phenotype characteristic of a plant, said method comprising introducing into a plant cell an exogenous nucleic acid comprising a nucleotide sequence encoding a polypeptide having 85% or greater sequence identity to an amino acid sequence in the Sequence Listing, wherein a plant produced from said plant cell differs in a plant growth or phenotype characteristic as compared to a control plant that does not comprise said nucleic acid.

18. The method of claim 17, further comprising the step of selecting one or more plants from a plurality of plants that have said difference in a plant growth or phenotype characteristic.

19. The method of any one of claim 17-18, wherein said exogenous nucleic acid is operably linked to a regulatory region.

20. The method of claim 19, wherein said regulatory region is a promoter.

21. A method of producing a plant tissue, said method comprising growing a plant cell comprising an exogenous nucleic acid comprising a nucleotide sequence according to claim 1, wherein said plant tissue has a plant growth or phenotype characteristic difference as compared to a corresponding control plant that does not comprise said nucleic acid.
